(19) United States
(12) Patent Application Publication
(10) Pub. No.: US 2024/0287585 A1
(43) Pub. Date: Aug. 29, 2024

(54) SYSTEMS, DEVICES AND METHODS FOR ANALYSIS

(71) Applicants: Anastasia SEVOSTIYANOVA, North Haven, CT (US); Mark FIANDACA, North Branford, CT (US); Ancera, Inc., Branford, CT (US)

(72) Inventors: Anastasia SEVOSTIYANOVA, North Haven, CT (US); Mark FIANDACA, North Branford, CT (US); Arjun GANESAN, Newington, CT (US); John C. VOYTA, Berlin, MA (US); Lisa LEIER, Hernando, MS (US)

(21) Appl. No.: 18/015,034

(22) PCT Filed: Jul. 14, 2021
(86) PCT No.: PCT/US2021/041616

§ 371 Date: Jan. 6, 2023

Related U.S. Application Data
(60) Provisional application No. 63/188,987, filed on May 14, 2021.
(60) Provisional application No. 63/051,712, filed on Jul. 14, 2020.

(51) Int. Cl. C12Q 1/6825; B01L 3/00; C12Q 1/6841; C12Q 1/6876; G01N 21/64

(52) U.S. Cl. CPC C12Q 1/6825; B01L 3/502761; C12Q 1/6841; C12Q 1/6876; G01N 21/6456; B01L 2300/0861; B01L 2400/043; G01N 2021/6432

(57) ABSTRACT

The subject disclosure presents, in some embodiments, is systems, devices and methods for multi-sample analyte analysis. In some such embodiments, a method is presented which includes labeling at least one target analyte in each of a plurality of individual samples with at least one unique label probe. The method further includes combining the plurality of individual samples into a mixture, and flowing the mixture within at least one fluidic channel/lane having arranged thereon or in fluid communication with at least one set of a plurality of spatially distinct capture zones. Embodiments of the present disclosure additionally relate to, among other things, systems, devices, and related methods for operating the same, to analyze and rapidly identify and quantify microbial pathogens in environmental, food, and clinical settings. Specifically, a rapid one-step FISH method where fixation, permeabilization and addition of fluorescent labeled probe all occur in one step on a ferrofluidic system.

### CROSS-REFERENCE TO RELATED APPLICATIONS

This application claims the benefit of and priority to U.S. Provisional Application Nos. 63/051,712, filed Jul. 14, 2020, and 63/188,987 filed May 14, 2021, the contents of each of which are hereby incorporated by reference in their entireties.

The current disclosure is additionally related to U.S. Pat. Nos. 8,961,878, and 9,999,855, and PCT publication nos. WO2014/144340, WO2014/144782, WO2014/144810, WO2014/145765, WO2014/165317, WO2016/210348, WO2017/004595, WO2018/026605, and WO2019/117877. Each of the foregoing disclosures incorporated by reference herein in its entirety.

### FIELD OF THE DISCLOSURE

The present disclosure is directed to various assays for multi-sample analyte analysis methods which includes labeling at least one target analyte in each of a plurality of individual samples with at least one unique label probe. The present disclosure is additionally directed to a fluorescent in-situ hybridization (FISH) assay in a ferrofluid-based microfluidic system that enables rapid identification and enumeration of target analytes (e.g. microbial pathogens).

### BACKGROUND

Measuring a single sample for the presence/absence of multiple target analytes and the ability to quantitatively determine the number of each target analyte in a sample remains a fundamental problem.

The methods of the present invention address this fundamental problem and further provide quantification without an additional step of re-testing the original sample.

When large numbers of samples are being tested, often the samples are pooled and tested as a combined sample. This is common when the number of positive samples is very low and the lab is only expecting occasional positive pools. When a positive combined sample is identified, each individual sample in that mixture is retested to identify the original positive sample or lot. These types of sample pooling strategies are not suitable when the number of positive samples is high.

Additionally, using fluorescent in-situ hybridization (FISH) for identification of bacterial pathogens has been reported in the literature, but the protocols require lengthy and cumbersome workflows which limit their commercial application.

Conventional bacterial labeling by FISH requires a two-stage fixation/permeabilization process followed by hybridization and washing steps prior to detection. Treatment of samples with a fixative agent preserves delicate cellular structures and target molecules for the remainder of the FISH process. Organic chemical fixatives are sometimes used in aqueous solutions, such as butanol or acetone. Formaldehyde is the classic fixation solution but is often replaced by paraformaldehyde which has less odor and is used at lower, less toxic concentrations. A stabilized form of aqueous formaldehyde known as formalin is used in fixation of tissue blocks for histological investigation.

After the fixation step, cells are permeabilized often with a multi-step process whereby the cells are treated with organic solvents that disrupt the lipid membrane barrier of the bacterial cell to allow free diffusion of the molecular probes into the cell during the subsequent steps. Often, particularly when analyzing samples where the target organism is a Gram-positive bacteria, the permeabilization step must be augmented with an enzymatic treatment, using lysozyme or lysostaphin, to further breakdown cell membranes. Typical permeabilization methods may involve a series of alcohol treatments starting with 90% ethanol and ending with 100% methanol. Detergents are also common components of permeabilization solutions. Often samples are heated during fixation and permeabilization to dehydrate them, denature proteins and nucleic acids within the cell and break down extracellular matrices.

After fixation and permeabilization steps, the cell labeling process of FISH starts: first with hybridization of the cells with a solution containing fluorescent labeled probe, often at a tightly controlled temperature; washing away the non-hybridized probe; and, finally, visualizing of the samples containing fluorescence-labeled bacteria under a fluorescent microscope, one at a time. In addition to the workflow challenges, the interpretation of the results of the conventional FISH assay is highly subjective and requires extensive personal training and specialized equipment.

When developing an assay for a pathogen, a broad specificity polyclonal antibody (or other binder) can be used to capture the target pathogen in a sample. Often these binders will also bind related pathogens that may share surface structures recognized by the binder, so simply labeling the captured cells may not have the accuracy required. Toward solving this issue and to increase the accuracy of this type of assay and eliminate the likelihood of false positive results, the method described herein labels the target pathogen using a fluorescence in-situ hybridization (FISH) technique using fluorescence labeled, sequence specific nucleic acid probes. After hybridizing the FISH probes, ferrofluid is flushed into the capture/imaging area and the captured, labeled cells are counted.

Given the encumbrances inherent with the current standard, the rapid, one-step FISH method described in this application is a significant improvement over traditional FISH methods.

### SUMMARY

In some embodiments of the present disclosure, a multi-sample analyte analysis method is provided and includes labeling at least one target analyte in each of a plurality of individual samples with at least one unique label probe. Each unique label probe is configured to bind with a first specific binding agent of a respective target analyte for a respective sample, and each respective first specific binding agent comprises at least one of an antibody, aptamer, lectin, and nucleic acid probe configured to bind to a respective target analyte of a respective sample, and/or is labeled with at least one of a unique synthetic oligonucleotide probe, a protein nucleic acid (PNA) probe, and any other synthetic polynucleotide analog configured for identification of an associated respective sample. The method further includes combining the plurality of individual samples into a mixture and flowing the mixture within at least one fluidic channel having arranged thereon or in fluid communication with at least one set of a plurality of spatially distinct capture zones. Each zone is configured with at least one unique oligonucleotide- or PNA-probe complementary to a particular unique label probe of a specific target analyte of at least one specific sample so as to capture the specific target analyte of the at least one specific sample, such that, each distinct capture zone is configured to capture a specific target analyte of the at least one specific sample.

Some embodiments of the disclosure herein can be carried out on assay systems and corresponding cartridges like those disclosed in PCT publication no. WO2018/026605 (“'605 PCT”), entitled, “Multilayered Disposable Cartridge for Ferrofluid-Based Assays and Methods of Use” (see, e.g., paragraph [0019]-[0027], FIGS. 1-4) which is incorporated by reference herein in its entirety.

In some embodiments, any cartridge, lane, system discussed herein can correspond to a cartridge, lane, and/or system of Ancera LLC's PIPER cartridge/system.

Such embodiments may further include one and/or another, and in some embodiments, a plurality of, and in some embodiments, all of (if not mutually exclusive), the following steps, features, functionality, structure, and/or clarification, yielding yet further embodiments:
incubating each sample with a first specific binding agent, where incubating can be performed prior to combining the plurality of individual samples into the mixture, and where incubation optionally includes diluting each sample at least once prior to incubation;
washing each sample, where:
   washing can be performed prior to combining the plurality of individual samples into the mixture;
   the labeled target analyte can comprise biological cells, such that, the labeled target cells prior to washing can be suspended in phosphate buffered saline or other compatible buffer;
   washing can comprise:
      centrifuging the labeled target analyte,
      removing supernatant, and
      re-suspending the labeled target analyte in phosphate buffered saline or other compatible buffer.
      Repeat wash when necessary
Combining the labeled, washed samples;
flowing an aliquot of the combined samples across the set of capture zones;
labeling the captured analyte with a fluorescent labeled target specific agent (labeled binder, FISH labeling, etc)
each analyte specific fluorescent labeled agent of the plurality can be configured to bind with a respective specific target analyte of a respective sample, and
each analyte specific fluorescent labeled agent of the plurality can comprise at least one of a fluorescent labeled antibody, aptamer, lectin, oligonucleotide probe, PNA probe, and any analyte specific reagent capable of binding to any portion of a respective specific target analyte;
the at least one analyte specific fluorescent labeled agent is configured to bind with a respective specific target analyte of a respective sample;
alternatively, the mixture or the labeled at least one target analyte in each of the plurality of individual samples, is further labeled with at least one analyte specific fluorescent labeled agent prior to flowing the mixture across the set of capture zones, and/or prior to combining the individual samples into the mixture;
exposing at least one set of capture zones of at least one channel to at least one fluorescing wavelength of light/radiation, where the wavelength configured to cause at least one analyte specific fluorescent labeled agent bound to a respective specific target analyte of a respective capture zone and sample to fluoresce;
exposing at least one set of capture zones of at least one channel to a plurality of fluorescing wavelengths of light/radiation, each respective wavelength configured to cause a respective analyte specific fluorescent labeled agent bound to a respective specific target analyte of a respective capture zone and sample to fluoresce;
imaging at least one set of capture zones of at least one channel to produce at least one image thereof, where imaging at least one set of capture zones comprises producing at least one image for each wavelength exposed to the at least one set of capture zones;
analyzing at least one image to determine a number of captured specific target analytes for each zone/sample;
analyzing the capture zones to determine a number of captured specific target analytes for each zone/sample;
each respective first binding agent may comprise an antibody, such that, antibody and target analyte form an antibody:target-analyte complex (ATAC);
treating each sample with a crosslinker (either before or after washing, in some embodiments, preferably after washing);
and
crosslinking is performed with a crosslinking agent;
   the crosslinking agent can comprise glutaraldehyde or a homo- or hetero-bifunctional cross-linking agent.

In some embodiments, a multi-sample analyte analysis method is provided and includes labeling at least one target analyte in each of a plurality of individual samples with at least one unique label probe, where each unique label probe is configured to bind with a first specific binding agent of a respective target analyte for a respective sample, and each respective first specific binding agent comprises at least one of an antibody, aptamer, lectin, and nucleic acid probe configured to bind to a respective target analyte of a respective sample, and/or is labeled with at least one of a unique synthetic oligonucleotide probe, a protein nucleic acid (PNA) probe, and any other synthetic polynucleotide analog configured for identification of an associated respective sample. The method also includes incubating each sample, wherein incubation optionally includes diluting each sample at least once prior to incubation, optionally washing each sample, optionally treating each washed sample with a crosslinker, combining the plurality of individual samples into a mixture, and flowing the mixture within at least one fluidic channel having arranged thereon or in fluid communication with at least one set of a plurality of spatially distinct capture zones. Each zone is configured with at least one unique oligonucleotide- or PNA-probe complementary to a particular unique label probe of a specific target analyte of at least one specific sample so as to capture the specific target analyte of the at least one specific sample, such that, each distinct capture zone is configured to capture a specific target analyte of the at least one specific sample. The method also includes flowing at least one analyte specific fluorescent labeled agent across the set of capture zones. Each unique fluorescent labeled probe of the plurality is configured to bind with a respective specific target analyte of a respective sample, and comprises at least one of a fluorescent labeled antibody, aptamer, lectin, oligonucleotide probe, PNA probe, and any analyte specific reagent capable of binding to any portion of a respective specific target analyte. The method further includes exposing the set of capture zones to at least one fluorescing wavelength of light/radiation configured to cause at least one unique fluorescent labeled probe bound to a respective specific target analyte of a respective capture zone and sample to fluoresce, or a plurality of fluorescing wavelengths of light/radiation configured to cause respective analyte specific fluorescent labeled probes corresponding to a respective fluorescing wavelength and bound to a respective specific target analyte of a respective capture zone and sample to fluoresce, imaging at least one set of capture zones for at least one channel during such fluorescing to produce at least one image thereof, and analyzing the at least one image, or a plurality of image each corresponding to a specific fluorescing wavelength, to determine a number of captured specific target analytes for each zone/sample.

In some embodiments, a single use, multiple-sample analyte analysis cartridge is presented, and includes a housing configured for insertion and removal from an assay system, and a plurality of fluidic channels arranged along at least a portion of the housing, each fluidic channel configured to receive at least one flow. The at least one flow comprises a mixture of a plurality of individual samples, and each of the individual samples have specific target analytes therein labeled with a unique label probe. Each unique label probe is configured to bind with a first specific binding agent of a respective target analyte for a respective sample, and each respective first specific binding agent comprises at least one of an antibody, aptamer, lectin, and nucleic acid probe configured to bind to a respective target analyte of a respective sample, and/or is labeled with at least one of a unique synthetic oligonucleotide probe, a protein nucleic acid (PNA) probe, and any other synthetic polynucleotide analog configured for identification of an associated respective sample. The cartridge also includes at least one set of a plurality of spatially distinct capture zones arranged along at least one fluidic channel, where each zone is configured with at least one unique oligonucleotide- or PNA-probe complementary to a particular unique label probe of a specific target analyte of at least one specific sample so as to capture the specific target analyte of the at least one specific sample, such that, each distinct capture zone is configured to capture a specific target analyte of the at least one specific sample.

Such embodiments may further include one and/or another, and in some embodiments, a plurality of, and in some embodiments, all of (if not mutually exclusive), the following steps, features, functionality, structure, and/or clarification, yielding yet further embodiments:
a plurality of windows, each configured to view at least a portion of a set of the plurality of capture zones, or each respective window configured to view at least a portion of a respective capture zone of a set of capture zones, where each window can be arranged adjacent a respective capture zone;
the cartridge can be configured such that at least one of each set of capture zones and each capture zone can be imaged;
the cartridge can be configured such that at all sets of capture zones, and/or all captures zones are imaged;
incubating means can be configured to incubate at least one of the samples, incubate a plurality of the samples, or incubate all of the samples, prior to mixing with other samples, where incubation can include diluting each sample at least once prior to incubation;
the cartridge is configured to include or receive at least one fluorescent labeling material or flow, respectively, comprising at least one analyte specific fluorescent labeled agent, where:
   each unique fluorescent labeled agent of the plurality can be configured to bind with a respective specific target analyte of a respective sample;
   each analyte specific fluorescent labeled agent of the plurality can comprise at least one of a fluorescent labeled antibody, aptamer, lectin, oligonucleotide probe, PNA probe, and any analyte specific reagent capable of binding to any portion of a respective specific target analyte;
the cartridge can be configured to receive at least one fluorescence excitation wavelength of light/radiation configured to cause at least one unique fluorescent labeled agent bound to a respective specific target analyte of a respective capture zone and sample to fluoresce;
the cartridge can be configured to use ferrofluid;
the cartridge can be configured to receive at least one fluorescence excitation wavelength of light/radiation configured to cause at least one unique fluorescent labeled agent bound to a respective specific target analyte of a respective capture zone and sample to fluoresce;
the cartridge can be configured to receive a plurality of fluorescence excitation wavelengths of light/radiation, each respective wavelength configured to cause a respective analyte specific fluorescent labeled agent bound to a respective specific target analyte of a respective capture zone and sample to fluoresce;
and
the cartridge can be configured for imaging at least one set of capture zones, a plurality of capture zones, or all of the capture zones for at least one set of capture zones, a plurality of sets of capture zones, or all sets of capture zones.

In some embodiments, a single use, multiple-sample analyte analysis cartridge is provided and includes a housing configured for insertion and removal from an assay system, and a plurality of fluidic channels arranged along at least a portion of the housing, each fluidic channel configured to receive at least one flow. The at least one flow comprises a mixture of a plurality of individual samples, and each of the individual samples have specific target analytes therein labeled with a unique label probe. Each unique label probe is configured to bind with a first specific binding agent of a respective target analyte for a respective sample, and each respective first specific binding agent comprises at least one of an antibody, aptamer, lectin, and nucleic acid probe configured to bind to a respective target analyte of a respective sample, and/or is labeled with at least one of a unique synthetic oligonucleotide probe, a protein nucleic acid (PNA) probe, and any other synthetic polynucleotide analog configured for identification of an associated respective sample. At least one set of a plurality of spatially distinct capture zones arranged along at least one fluidic channel, where each zone is configured with at least one unique oligonucleotide- or PNA-probe complementary to a particular unique label probe of a specific target analyte of at least one specific sample so as to capture the specific target analyte of the at least one specific respective sample, such that, each distinct capture zone is configured to capture a specific target analyte of the at least one specific sample. The cartridge also includes a plurality of windows, with each arranged adjacent a respective capture zone and configured to view at least a portion of a set of the plurality of capture zones, or each respective window configured to view at least a portion of a respective capture zone of a set of capture zones, such that at least one of each set of capture zones and each capture zone can be imaged.

In some embodiments, a multi-sample mixture assay system is provided which includes a receiving area and/or housing configured for receiving a cartridge according to any of the above-noted embodiments, a fluorescing means (e.g., a light source configured for wavelength specific fluorescence excitation, which may also include a filter configured to monitor resulting fluorescence emission from each fluorophore), a processor, and an imager. The cartridge is configured to flow a mixture through at least one of the fluidic channels, where the mixture comprises a plurality of individual samples, in which each sample is labeled with a unique label probe, such that each unique label probe is configured to bind with a first specific binding agent of a respective target analyte for a respective sample. Each respective first specific binding agent comprises at least one of an antibody, aptamer, lectin, and nucleic acid probe configured to bind to a respective target analyte of a respective sample, and/or is labeled with at least one of a unique synthetic oligonucleotide probe, a protein nucleic acid (PNA) probe, and any other synthetic polynucleotide analog configured for identification of an associated respective sample. Labeled target analytes of each sample binds to one and/or another of the capture zones of at least one set of capture zones of the at least one fluidic channels of the cartridge, and each zone being configured with at least one unique oligonucleotide- or PNA-probe complementary to a particular unique label probe of a specific target analyte of at least one specific sample so as to capture the specific target analyte of the at least one specific sample. The system is configured to flow a plurality of analyte specific fluorescent labeled agent across each set of capture zones, each analyte specific fluorescent labeled probe of the plurality is configured to bind with a respective specific target analyte of a respective sample, and comprises at least one of a fluorescent labeled antibody, aptamer, lectin, oligonucleotide probe, PNA probe, and any analyte specific reagent capable of binding to any portion of a respective specific target analyte. The fluorescing means exposes the capture zones of at least one set of capture zones to at least one fluorescence excitation wavelength of light/radiation configured to cause at least one unique fluorescent labeled agent bound to a respective specific target analyte of a respective capture zone and sample to emit fluorescence, the imager images the at least one set of capture zones during such fluorescing to produce at least one image thereof, and the processor is configured with computer instructions operating thereon to control the assay system and analyze the at least one image to determine a number of captured specific target analytes for each zone/sample.

In some embodiments, a multi-sample analyte analysis method is provided and comprises labeling at least one target analyte in each of a plurality of individual samples with a unique label probe and an analyte specific fluorescent labeled agent. Each unique label probe is configured to bind with a first specific binding agent of a respective target analyte for a respective sample, and each respective first specific binding agent comprises at least one of an antibody, aptamer, lectin, and nucleic acid probe configured to bind to a respective target analyte of a respective sample, and/or is labeled with at least one of a unique synthetic oligonucleotide probe, a protein nucleic acid (PNA) probe, and any other synthetic polynucleotide analog configured for identification of an associated respective sample. Each unique fluorescent labeled agent of the plurality is configured to bind with a respective specific target analyte of a respective sample, and comprises at least one of a fluorescent labeled antibody, aptamer, lectin, oligonucleotide probe, PNA probe, and any analyte specific reagent capable of binding to any portion of a respective specific target analyte. The method further includes, optionally incubating each sample, wherein incubation optionally includes diluting each sample at least once prior to incubation, optionally washing each sample, optionally treating each washed sample with a crosslinker, combining the plurality of individual samples into a mixture, and flowing the mixture within at least one fluidic channel having arranged thereon or in fluid communication with at least one set of a plurality of spatially distinct capture zones. Each zone is configured with at least one unique oligonucleotide- or PNA-probe complementary to a particular unique label probe of a specific target analyte of a respective specific sample so as to capture the specific target analyte of the at least one specific sample, such that, each distinct capture zone is configured to capture a specific target analyte of the at least one specific sample. The method further includes exposing the set of capture zones to at least one fluorescing wavelength of light/radiation configured to cause at least one unique fluorescent labeled probe bound to a respective specific target analyte of a respective capture zone and sample to fluoresce, imaging the set of capture zones during such fluorescing to produce at least one image thereof, and analyzing the at least one image to determine a number of captured specific target analytes for each zone/sample.

In some embodiments, a multi-sample analyte analysis method is provided and includes labeling at least one target analyte in each of a plurality of individual samples with at least one unique label probe. Each unique label probe is configured to bind with a first specific binding agent of a respective target analyte for a respective sample, and each respective first specific binding agent comprises at least one of an antibody, aptamer, lectin, and nucleic acid probe configured to bind to a respective target analyte of a respective sample, and/or is labeled with at least one of a unique synthetic oligonucleotide probe, a protein nucleic acid (PNA) probe, and any other synthetic polynucleotide analog configured for identification of an associated respective sample. The method also includes combining the plurality of individual samples into a first mixture and exposing the first mixture to a plurality of sets of capture particles, thereby forming a second mixture.

In some embodiments:
the capture particles (e.g., beads) are labeled with one or more fluorophores at different ratios to prepare sets of unique particles;
different size particles can be used, which also may be labeled with one or more fluorophores;
at least one unique particle per individual sample; and/or
non-fluorescent, different sized capture particles can be used.

In some embodiments, an assay based on a capture particle (e.g., containing at least one unique fluorophore as well as a unique oligo- or PNA-label probe), for an immunoassay, or a probe, hybridization-based assay on individual samples. Results can be had by fluorescence-activated cell sorting (FACS), and/or by capture in a coded universal cartridge. Thus, instead of processing individual samples with a probe labeled binder to label cells in a sample, a binder can be attached to the capture particle and use the binder to bind to an analyte of interest (in some embodiments, wash if necessary, and/or crosslink if necessary). Accordingly, multiple samples can be combined, and further fluorescent labeled binders can be used to label a target of interest (again, results could be obtained via at least one of FACS and universal assay cartridge).

Each set of capture particles is configured with at least one unique oligonucleotide- or PNA-probe complementary to a particular unique label probe of a specific target analyte of at least one specific sample so as to capture the specific target analyte of the at least one specific sample, such that, each set of respective capture particles is configured to capture a specific target analyte of the at least one specific sample.

Such embodiments may further include one and/or another, and in some embodiments, a plurality of, and in some embodiments, all of (if not mutually exclusive), the following steps, features, functionality, structure, and/or clarification, yielding yet further embodiments:
incubating each sample with a first specific binding agent;
incubation can include diluting each sample at least once prior to incubation;
washing each sample:
   where washing can be performed prior to combining the plurality of individual samples into the first mixture,
   the labeled target analyte comprises biological cells, and the labeled target cells prior to washing being suspended in phosphate buffered saline or other compatible buffer; and/or
   washing comprises, centrifuging the labeled target analyte, removing supernatant, and re-suspending the labeled target analyte in phosphate buffered saline or other compatible buffer.
   The washing steps can be repeated
Combining each labeled, washed sample
exposing at least one of the first mixture and the second mixture to the sets of capture particles to at least one analyte specific fluorescent labeled agent, where each analyte specific fluorescent labeled agent of the plurality is configured: to bind with a respective specific target analyte of a respective sample, and comprises at least one of a fluorescent labeled antibody, aptamer, lectin, oligonucleotide probe, PNA probe, and any analyte specific reagent capable of binding to any portion of a respective specific target analyte;
the at least one analyte specific fluorescent labeled agent is configured to bind with a respective specific target analyte of a respective sample;
exposing second mixture to at least one fluorescing wavelength of light/radiation, the wavelength configured to cause at least one analyte specific fluorescent labeled agent bound to a respective specific target analyte of a respective set of capture particles to fluoresce;
exposing the second mixture, optionally via flow cytometry, to a plurality of fluorescing wavelengths of light/radiation, each respective wavelength configured to cause a respective analyte specific fluorescent labeled probe bound to a respective specific target analyte of a respective capture particle of at least one set of capture particles to fluoresce;
optionally, prepare a plot of the number of cells versus capture particles wherein each captured particle applies to the original sample identification;
imaging the capture particles of the second mixture to produce at least one image thereof;
imaging the capture particles of the second mixture comprises producing at least one image for each wavelength exposed to the at least one set of capture zones;
analyzing the capture particles of the second mixture so as to determine a number of captured specific target analytes for each sample;
analyzing the at least one image to determine a number of captured specific target analytes for each sample;
treating each sample with a crosslinker, or treating each washed sample with a crosslinker.
and
each respective first binding agent comprises an antibody, such that, antibody and target analyte form an antibody:target-analyte complex (ATAC), and after washing, the method further comprises crosslinking the ATAC, where crosslinking can be performed with a crosslinking agent, where the crosslinking agent comprises glutaraldehyde or a homo- or hetero-bifunctional cross-linking agent.

Accordingly, in some embodiments, multiple combined samples, composed of individual samples pre-labeled with a known analyte specific antibody, can be pre-screened by any assay (immunoassay, polymerase-chain-reaction (PCR) assay, sequencing assay, and the like) to determine if any of the combined samples contains a target analyte of interest. After such screening, the positive combined samples can then be processed such that it is flowed over sample specific capture zones of an assay device to determine the identity of an individual sample that contains the target analyte of interest.

In some embodiments, a multi-sample analyte analysis method is provided and includes labeling at least one target analyte in each of a plurality of individual samples with at least one unique label probe, where each unique label probe is configured to bind with a first specific binding agent of a respective target analyte for a respective sample, and each respective first specific binding agent comprises at least one of an antibody, aptamer, lectin, and nucleic acid probe configured to bind to a respective target analyte of a respective sample, and/or is labeled with at least one of a unique synthetic oligonucleotide probe, a protein nucleic acid (PNA) probe, and any other synthetic polynucleotide analog configured for identification of an associated respective sample. The method further includes incubating each sample, wherein incubation optionally includes diluting each sample at least once prior to incubation, optionally washing each sample, optionally treating each washed sample with a crosslinker, combining the plurality of individual samples into a first mixture, and exposing the first mixture to at least one set of capture particles forming a second mixture. Each set of capture particles is configured with at least one unique oligonucleotide- or PNA-probe complementary to a particular unique label probe of a specific target analyte of at least one specific sample so as to capture the specific target analyte of the at least one specific sample, such that, each distinct set of capture particles is configured to capture a specific target analyte of the at least one specific sample. The method further includes exposing at least one of the first mixture and second mixture to at least one analyte specific fluorescent labeled agent, where each unique fluorescent labeled agent of the plurality is configured to bind with a respective specific target analyte of at least one sample, and comprises at least one of a fluorescent labeled antibody, aptamer, lectin, oligonucleotide probe, PNA probe, and any analyte specific reagent capable of binding to any portion of a respective specific target analyte. The method further yet includes exposing the second mixture, optionally via flow cytometry, to at least one fluorescing wavelength of light/radiation configured to cause at least one unique fluorescent labeled agent bound to a respective specific target analyte to fluoresce, or a plurality of fluorescing wavelengths of light/radiation configured to cause respective analyte specific fluorescent labeled agent corresponding to a respective fluorescing wavelength and bound to a respective specific target analyte to fluoresce. The at least one set of the capture particles are analyzed, optionally via flow cytometry, during such fluorescing to produce at least one image thereof, and the at least one image, or a plurality of images, each corresponding to a specific fluorescing wavelength, are analyzed to determine a number of at least one set of capture particles that have captured specific target analytes.

These embodiments and others, as well as other objects and advantages of the various embodiments of the current disclosure are even more clear with reference to the following detailed description (and any associated figures).

Embodiments of the present disclosure relate to, among other things, systems, devices, and related methods for operating the same, a rapid one-step fluorescent in-situ hybridization (FISH) method where fixation, permeabilization and addition of fluorescent labeled probe all occur in a single step.

In one aspect, the present disclosure is directed to a rapid one-step FISH method which occurs on a ferrofluid-based microfluidic system that has been designed to identify and enumerate pathogens. In some embodiments, the method of the present disclosure is a more streamlined approach compared to traditional FISH assays. In some embodiments, the rapid one-step FISH method is used for detecting pathogens in the food processing industry; rapid human and veterinary clinical diagnostics; monitoring diseases; monitoring circulating tumor cells; fermentation processes; water quality; evaluations of soil microbiome and soil health; and environmental monitoring, among others.

In some embodiments, the ferrofluid-based microfluidic system uses ferrofluid to push and focus one or more target analytes into a cell binder capture zone of a cartridge lane to capture the target analyte. In some embodiments, the method described herein labels the target pathogen using a fluorescence in-situ hybridization (FISH) technique which employs fluorescence labeled, sequence specific nucleic acid probes. In some embodiments, ferrofluid is used to flow the analyte into capture/imaging area, the analyte binds to the capture/imaging area, the FISH probes are flowed into the capture/imaging area, the probe and analyte are allowed to hybridize, ferrofluid is used to remove excess probe from the capture/imaging area and the labeled analyte is counted.

In some embodiments, after hybridizing the FISH probes with the target analyte, ferrofluid is used to flow the hybridized analyte into capture/imaging area and wash excess probe out of the capture/imaging area prior to counting the labeled cells.

In one aspect, the present disclosure is directed to a method for performing a ferrofluid-based microfluidic fluorescence in-situ hybridization assay comprising:
preparing a first mixture of ferrofluid and a sample containing or suspected of containing a target analyte;
flowing the first mixture through a channel comprising a capture region while applying a magnetic field, wherein the capture region comprises one or more binding agents which capture the target analyte;
flowing the second solution which comprises a labeled probe configured to hybridize with the target analyte into the channel;
raising the temperature of the channel to the probe hybridization temperature; allowing the probe to hybridize with the sample;
flowing the ferrofluid through the channel to remove the unbound probe;
measuring for the presence or absence of the label.

In some embodiments, the method further comprises counting the number of labeled target analytes. In some embodiments, the target analyte is a cell.

In one aspect, the present disclosure is directed to a method for performing a ferrofluid-based microfluidic fluorescence in-situ hybridization assay comprising:
preparing a first mixture of ferrofluid and a sample containing or suspected of containing a target analyte;
providing a second mixture comprising a probe, wherein the probe comprises a label and the probe is configured to hybridize with the target analyte;
combining at least the first mixture and the second mixture, wherein the probe is allowed to hybridize with the target analyte at the probe hybridization temperature;
flowing the combined mixture through a channel comprising a capture region while applying a magnetic field, wherein the capture region comprises one or more binding agents which capture the target analyte;
applying a magnetic field to flow the ferrofluid through the channel to remove unbound probe;
measuring for the presence or absence of the label.

In some embodiments, the method further comprises counting the number of labeled target analytes. In some embodiments, the target analyte is a cell.

In one aspect, the present disclosure is directed to a method for performing a ferrofluid-based microfluidic fluorescence in-situ hybridization assay comprising:
preparing a first mixture of ferrofluid and a sample containing or suspected of containing a target analyte;
flowing the first mixture through a channel comprising a capture region while applying a magnetic field, wherein the capture region comprises one or more binding agents which capture the target analyte;
flowing a second solution into the channel, wherein the second solution comprises a labeled probe configured to hybridize with the target analyte;
raising the temperature of the channel to the probe hybridization temperature; allowing the probe to hybridize with the sample;
flowing the ferrofluid through the channel to remove the unbound probe;
measuring for the presence or absence of the label.

In one aspect, the present disclosure is directed to a method for performing a ferrofluid-based microfluidic fluorescence in-situ hybridization assay comprising:
preparing a first mixture of ferrofluid and a sample containing or suspected of containing a target analyte;
combining at least the first mixture and a second mixture comprising a labeled probe configured to hybridize with the target analyte, wherein the probe is allowed to hybridize with the target analyte at the probe hybridization temperature;
flowing the combined mixture through a channel comprising a capture region while applying a magnetic field, wherein the capture region comprises one or more binding agents which capture the target analyte;
applying a magnetic field to flow the ferrofluid through the channel to remove unbound probe;
measuring for the presence or absence of the label.

In one aspect, the present disclosure is directed to a method of fluorescence in-situ hybridization detection of an analyte in a sample comprising:
providing a sample comprising or suspected of comprising a target analyte;
providing a fluorescent label probe, wherein said fluorescent label probe comprises a binding sequence, configured to hybridize with the target analyte;
combining at least the sample, the fluorescent label probe, and a buffer to form a mixture;
incubating the mixture at the probe hybridization temperature;
adding the mixture to a ferrofluid-based microfluidic device;
flowing the ferrofluid and moving the mixture toward a capture region while applying a magnetic field, wherein the capture region comprises one or more binding agents which capture the target analyte, and a detector;
detecting the presence or absence of the target analyte.

In some embodiments, the method further comprises counting the number of labeled target analytes. In some embodiments, the target analyte is a cell.

In one aspect, the present disclosure is directed to a method of fluorescence in-situ hybridization detection of an analyte in a sample comprising:
providing a sample comprising or suspected of comprising a target analyte;
providing a fluorescent label probe, wherein said fluorescent label probe comprises a binding sequence, configured to hybridize with the target analyte;
combining at least the sample, the fluorescent label probe, and a buffer to form a mixture;
incubating the mixture at the probe hybridization temperature;
adding the mixture to a ferrofluid-based microfluidic device;
flowing the ferrofluid and moving the mixture toward a detector region;
detecting the presence or absence of the target analyte.

In some embodiments, the method further comprises counting the number of labeled target analytes. In some embodiments, the target analyte is a cell.

In some embodiments, the method of the present disclosure comprises a label.

In some embodiments, the second solution is prepared from a lyophilized starting material.

In some embodiments, the second solution is prepared from a lyophilized reagent.

some embodiments, the second solution is prepared from a lyophilized starting material and a solvent.

In some embodiments, the second solution is prepared from a lyophilized reagent and a reconstitution solution (e.g. water). In some embodiments, the second solution is prepared from a lyophilized reagent and an aqueous reconstitution solution.

In some embodiments, the method of the present disclosure comprises a label, wherein the label is fluorescent.

In some embodiments, the method of the present disclosure comprises a probe.

In some embodiments, the method of the present disclosure comprises a probe, wherein the probe is a PNA-based probe.

In some embodiments, the method of the present disclosure comprises a probe, wherein the probe is an RNA-based probe.

In some embodiments, the method of the present disclosure comprises a probe, wherein the probe is an DNA-based probe.

In some embodiments, the method of the present disclosure comprises a fluorescent labeled probe which further comprises a quenching moiety which is released upon hybridizing with the target analyte.

In some embodiments, the method of the present disclosure further comprises the addition of a helper probe, which assists the hybridization of the fluorescent labeled probe with the target analyte.

In some embodiments, the method of the present disclosure comprises a ferrofluid, wherein the ferrofluid is a biologically compatible ferrofluid.

In some embodiments, the method of the present disclosure comprises a ferrofluid, wherein the ferrofluid is a biologically compatible PEGylated ferrofluid.

In some embodiments, the method of the present disclosure comprises a ferrofluid, wherein the ferrofluid is a biologically compatible surfactant stabilized ferrofluid.

In some embodiments, the method of the present disclosure measures an analyte.

In some embodiments, the method of the present disclosure measures an analyte, wherein the analyte is a bacterial cell.

In some embodiments, the method of the present disclosure measures an analyte, wherein the analyte is a gram-positive bacterial cell.

In some embodiments, the method of the present disclosure measures an analyte, wherein the analyte is a gram-negative bacterial cell.

In some embodiments, the method of the present disclosure comprises one or more mixtures, wherein the one or more mixtures comprise Polymyxin B.

In some embodiments, the method of the present disclosure comprises a quencher dye

In some embodiments, the method of the present disclosure comprises a quencher dye, wherein the quencher dye is used to decrease non-specific signal in the ferrofluid-based FISH assay.

In some embodiments, the method of the present disclosure comprises helper probe

In some embodiments, the method of the present disclosure comprises a helper probe, wherein the helper probe is added to the buffer.

In some embodiments, the method of the present disclosure comprises a hybridization time,

In some embodiments, the method of the present disclosure comprises a hybridization time, wherein the hybridization time is about 5 minutes or less, about 10 minutes or less, about 15 minutes or less, about 20 minutes or less, about 25 minutes or less, about 30 minutes or less, about 35 minutes or less, about 40 minutes or less, about 45 minutes or less, about 50 minutes or less, about 55 minutes or less, about 60 minutes or less.

In some embodiments, the method of the present disclosure comprises a hybridization time, wherein the hybridization time is from about 5 minutes to about 60 minutes, from about 10 minutes to about 60 minutes, from about 15 minutes to about 60 minutes, from about 20 minutes to about 60 minutes, from about 25 minutes to about 60 minutes, from about 30 minutes to about 60 minutes, from about 35 minutes to about 60 minutes, from about 40 minutes to about 60 minutes, from about 45 minutes to about 60 minutes, from about 50 minutes to about 60 minutes, from about 55 minutes to about 60 minutes.

In some embodiments, the method of the present disclosure comprises a hybridization time, wherein the hybridization time is from about 5 minutes to about 55 minutes, from about 10 minutes to about 55 minutes, from about 15 minutes to about 55 minutes, from about 20 minutes to about 55 minutes, from about 25 minutes to about 55 minutes, from about 30 minutes to about 55 minutes, from about 35 minutes to about 55 minutes, from about 40 minutes to about 55 minutes, from about 45 minutes to about 55 minutes, from about 50 minutes to about 55 minutes.

In some embodiments, the method of the present disclosure comprises a hybridization time, wherein the hybridization time is from about 5 minutes to about 50 minutes, from about 10 minutes to about 50 minutes, from about 15 minutes to about 50 minutes, from about 20 minutes to about 50 minutes, from about 25 minutes to about 50 minutes, from about 30 minutes to about 50 minutes, from about 35 minutes to about 50 minutes, from about 40 minutes to about 50 minutes, from about 45 minutes to about 50 minutes.

In some embodiments, the method of the present disclosure comprises a hybridization time, wherein the hybridization time is from about 5 minutes to about 45 minutes, from about 10 minutes to about 45 minutes, from about 15 minutes to about 45 minutes, from about 20 minutes to about 45 minutes, from about 25 minutes to about 45 minutes, from about 30 minutes to about 45 minutes, from about 35 minutes to about 45 minutes, from about 40 minutes to about 45 minutes.

In some embodiments, the method of the present disclosure comprises a hybridization time, wherein the hybridization time is from about 5 minutes to about 40 minutes, from about 10 minutes to about 40 minutes, from about 15 minutes to about 40 minutes, from about 20 minutes to about 40 minutes, from about 25 minutes to about 40 minutes, from about 30 minutes to about 40 minutes, from about 35 minutes to about 40 minutes.

In some embodiments, the method of the present disclosure comprises a hybridization time, wherein the hybridization time is from about 5 minutes to about 35 minutes, from about 10 minutes to about 35 minutes, from about 15 minutes to about 35 minutes, from about 20 minutes to about 35 minutes, from about 25 minutes to about 35 minutes, from about 30 minutes to about 35 minutes.

In some embodiments, the method of the present disclosure comprises a hybridization time, wherein the hybridization time is from about 5 minutes to about 30 minutes, from about 10 minutes to about 30 minutes, from about 15 minutes to about 30 minutes, from about 20 minutes to about 30 minutes, from about 25 minutes to about 30 minutes.

In some embodiments, the method of the present disclosure comprises a hybridization time, wherein the hybridization time is from about 5 minutes to about 25 minutes, from about 10 minutes to about 25 minutes, from about 15 minutes to about 25 minutes, from about 20 minutes to about 25 minutes.

In some embodiments, the method of the present disclosure comprises a hybridization time, wherein the hybridization time is from about 5 minutes to about 20 minutes, from about 10 minutes to about 20 minutes, from about 15 minutes to about 20 minutes.

In some embodiments, the method of the present disclosure comprises a hybridization time, wherein the hybridization time is from about 5 minutes to about 15 minutes, from about 10 minutes to about 15 minutes.

In some embodiments, the method of the present disclosure comprises a hybridization time, wherein the hybridization time is from about 5 minutes to about 10 minutes.

In some embodiments, the sample is incubated in media comprising glucose prior to analysis in any of the methods disclosed herein.

In some embodiments, the sample is incubated in media comprising pyruvate prior to analysis in any of the methods disclosed herein.

In some embodiments, the sample is incubated in media comprising a mixture of glucose and pyruvate prior to analysis in any of the methods disclosed herein.

In some embodiments, the sample is incubated in media comprising 200 mM glucose and 20 mM pyruvate prior to analysis in any of the methods disclosed herein.

In some embodiments, the sample is incubated in media for about 15 minutes, about 30 minutes, about 45 minutes, or about 60 minutes in any of the methods disclosed herein.

In some embodiments, the sample is incubated in media at about 35° C., about 40° C., about 42° C., or about 45° C. in any of the methods disclosed herein.

In some embodiments, the sample is incubated in media comprising 200 mM glucose and 20 mM pyruvate for about 30 minutes at about 42° C. prior to analysis.

In one aspect, the present disclosure is directed to a kit for performing a ferrofluid-based microfluidic fluorescent in-situ hybridization assay comprising:
a cartridge comprising a plurality of micro-channels;
an inlet receiving a ferrofluid, biological cell mixture, and a label probe, the inlet in communication with at least one of the plurality of micro-channels;
a magnetic field source arranged proximate to at least one of the inlet and the plurality of micro-channels;
at least one capture region having one of more binding agents for capturing at least a first type of analyte, the at least one capture region arranged proximate to a first portion of at least one of the micro-channels, wherein the first portion is downstream from the inlet;
a detection reagent comprising a fluorescent label probe, wherein said fluorescent label probe comprises a binding sequence, configured to hybridize with the target analyte.

In some embodiments, the kit of the present disclosure comprises any of the embodiments discussed regarding the method(s) of the present disclosure.

In some embodiments, the target analytes (eg: pathogenic microbes) in a sample can be labeled with the described rapid FISH method prior to analyzing the sample. Analysis of the sample can be performed after mixing the sample with ferrofluid and then processing the sample/ferrofluid using a similar binder modified cartridge as described above, or in a cartridge that does not contain a capture region. Alternatively, the labeled sample could be analyzed by other methods such as traditional fluorescence microscopy, or fluorescence activated cell sorting.

The above noted embodiments, as well as objects and advantages thereof, will become even more evident by the following detailed description, and corresponding drawings, a brief description of which is set out below.

### BRIEF DESCRIPTION OF THE FIGURES

FIG. 1 illustrates a flowchart demonstrating the working principle behind the assay system of the present disclosure.

FIG. 2 illustrates the cell barcoding schematic depicting six principal steps according to embodiments herein.

FIG. 3 shows the results obtained when the Barcode assay was tested using pre-stained cells.

FIG. 4A-C depicts independent cell detection within pooled samples: example with negative Sample A.

FIG. 5A-C depicts independent cell detection within pooled samples: example with negative sample B.

FIG. 6A-C depicts independent cell detection within pooled samples: example with negative sample C.

FIG. 7A-C depicts independent cell detection within pooled samples: example with negative sample D.

FIG. 8A-C depicts the analysis of Salmonella in poultry rinsate samples.

FIG. 9A illustrates a 1-step PNA FISH that results in bright and specific signal. The sequences of the Salmonella-specific probes and their target site are shown at the top of the figure. The composition the conventional PNA-FISH hybridization buffer and the result of the assay are shown in the middle (performed on fixed, permeabilized cells); and the composition of the 1-step FISH and the result of the assay are shown at the bottom (no fixation/permeabilization).

FIG. 9B illustrates the 1-step FISH method for Salmonella Typhimurium using various buffer formulations.

FIG. 9C illustrates images obtained when using the Salmonella-specific NestB probe to FISH label several Salmonella serotypes and one non-Salmonella bacteria that contains the complementary ribosomal RNA sequence to the probe (Salmonella Typhimurium, Salmonella Heidelberg, Salmonella Enteritidis, and Citrobacter sedlakii) as well as bacteria containing “mismatched” ribosomal RNA sequences (Proteus mirabilis, Klebsiella pneumoniae, Citrobacter freundii, E. coli). In the demonstration, polymyxin B was used as a permeabilization reagent for the FISH method, according to some embodiments.

FIG. 10 illustrates FISH labeling in a device, optionally Ancera LLC's PIPER cartridge/system. Specifically, Salmonella Typhimurium cells, or 5 million cells Citrobacter freundii cells, or 5 million cells Proteus mirabilis cells were mixed with PEG ferrofluid and loaded into the sample reservoir on the cartridge. The top 3 panels show labeling of all cells captured with the SYBR intercalating dye. The middle 3 panels illustrate FISH labeling with a fluorescent-probe that labels all bacteria (Bac-Uni-1). The bottom 3 panels illustrate specific labeling using the salmonella specific fluorescent-NEST-B probe (plus helper probes). Pre-labeled Salmonella positive control cells were included in the FISH probe labeling reactions and are indicated as examples in the last 2 images.

FIG. 11A illustrates the ferrofluid-based microfluidic device analysis of Salmonella Typhimurium.

FIG. 11B illustrates the ferrofluid-based microfluidic device analysis of Salmonella Arizonae with the NestF and helpers 1, 2. The lower window represents a control antibody which does not capture Salmonella.

FIG. 11C illustrates the ferrofluid-based microfluidic device analysis of Salmonella Arizonae.

FIG. 11D illustrates the ferrofluid-based microfluidic device analysis of Salmonella Newport with the NestF and helpers 1, 2. The lower window represents a control antibody which does not capture Salmonella.

FIG. 11E illustrates the ferrofluid-based microfluidic device analysis of Salmonella Newport.

FIG. 11F illustrates the ferrofluid-based microfluidic device analysis of Salmonella Muenchen with the NestF and helpers 1, 2. The lower window represents a control antibody which does not capture Salmonella.

FIG. 11G illustrates the ferrofluid-based microfluidic device analysis of Salmonella Muenchen.

FIG. 11H illustrates the ferrofluid-based microfluidic device analysis of Salmonella freundii.

FIG. 11I illustrates the ferrofluid-based microfluidic device analysis of E. coli.

FIG. 11J illustrates the ferrofluid-based microfluidic device analysis of Proteus mirabilis.

FIG. 11K illustrates the ferrofluid-based microfluidic device analysis of Klebsiella pneumoniae with the NestF and helpers 1, 2

FIG. 11L illustrates the ferrofluid-based microfluidic device analysis of Enterobacter cancerogenus.

FIG. 11M illustrates the ferrofluid-based microfluidic device analysis of Shigella sonnei.

FIG. 11N illustrates the results of Example #2A showing the ferrofluid-based microfluidic device analysis of Staphylococous saprophyticus.

FIG. 11O illustrates the ferrofluid-based microfluidic device analysis of Pseudomonas gragi.

FIG. 12 depicts a perspective view of an exemplary embodiment of a cartridge for the methods of the present disclosure.

FIG. 13 depicts inclusivity and exclusivity testing. Selected values of counts are shown in logarithmic scale. Inclusivity strains (Salmonella enterica serovars are shown in green; exclusivity strains are shown in red. Blue bar represents the average of counts from 11 lanes that had no bacteria added (blanks) and is reflective of combination of the system noise and imperfections in the image recognition algorithm used in this study.

FIG. 14 depicts the inclusivity strains used in validation. The serovars on the CDC list of the most epidemiologically important are noted with an asterisk.

FIG. 15 depicts the exclusivity strains used in validation.

FIG. 16 depicts the detection of Salmonella enterica subsp. enterica in dose-dependent manner. Ten-fold dilutions of several Salmonella serovars were prepared and processed using the FISH methods discussed herein.

FIG. 17 depicts the detection of Salmonella enterica subsp. enterica in dose-dependent manner. Ten-fold dilutions of several Salmonella serovars were prepared and processed using the FISH methods discussed herein.

FIG. 18 depicts a plot of Salmonella in common food matrices and environmental samples detected via the methods of the present disclosure in poultry rinse and ground turkey samples. Serial dilutions of 3 salmonella serotypes, S. Typhimurium, S. Enteritidis and S. Kentucky are depicted with cells diluted in media alone or the sample matrices.

FIG. 19 depicts a plot of serial dilutions of S. Heidelberg, S. Virginia and S. Weltevreden diluted in pure media and in ground turkey suspension (1 g in 25 ml media).

FIG. 20 depicts naturally occurring Salmonella in boot sock sample. Boot-sock sample was prepared as per Food Safety and Inspection Service (FSIS) guidelines using supplemented RapidChek media (Romer Labs) and enriched for 16 hours at 42° C. prior to testing. Image on top shows (Top) detection by the molecular probes. Reference method confirmed presence of Salmonella in the sample. (Bottom) testing the same sample without molecular probes. The observed signal is not due to autofluorescence frequently present in environmental samples, because omitting of the specific probes from the labeling solution did not yield a signal.

FIG. 21 depicts naturally occurring Salmonella in ground turkey sample. The sample was prepared as per FSIS guidelines using Rapid Chek media (Romer Labs) and enriched for 16 hours at 42° C. (Top) Sample positive for Salmonella, as confirmed by the reference method. (Bottom) Sample negative for Salmonella, as confirmed by the reference method.

FIG. 22 shows the complete design of one cartridge lane with a description of how a sample mixed with ferrofluid flows through the lane and the location of where cells are captured is noted. This capture zone aligns with cartridge analysis windows (106) in FIG. 12. Note that after capture of pathogens from the sample, the cartridge is processed with the FISH protocol described herein.

FIG. 23A-B depicts the effect of signal recovery treatment on (A) Salmonella Typhimurium grown for 11 hours as pure culture or (B) spiked into fecal sample (1 g chicken feces per 200 mL of BPW media). An aliquot of the culture was run using Ancera LLC's PIPER cartridge/systems is (left panels) or mixed 1:1 (v:v) with signal recovery media (glucose 200 mM, pyruvate 20 mM) and incubated at 42° C. for 30 min prior to run (right panels).

FIG. 24A-C depicts signal recovery treatment on Salmonella Typhimurium grown for 11 hours as pure culture, grey bars, or spiked into fecal sample (1 g chicken feces per 200 mL of BPW media), striped bars; (B) shows optimization of glucose and/or pyruvate levels in signal recovery media; (C) shows example images from the optimization experiment.

FIG. 25 depicts sequences of Salmonella probe, its target site on 16S rRNA and helper probes. SNP critical for bacteria discrimination are highlighted in green and red. E. coli number nomenclature used.

Accordingly, theses drawings, which are incorporated in and constitute a part of this specification, illustrate some of the exemplary embodiments of the present disclosure and together with the description, serve to explain specific principles of at least some embodiments of the disclosure.

### DETAILED DESCRIPTION OF AT LEAST SOME OF THE EMBODIMENTS

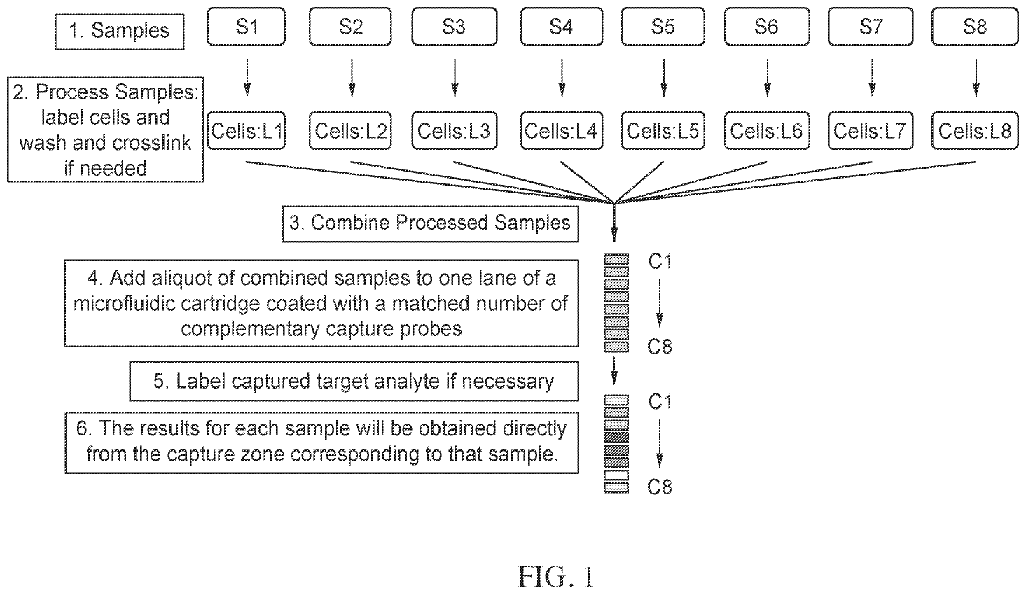

Some embodiments of the disclosure can be carried out on assay systems and corresponding cartridges like those disclosed in PCT publication no. WO2018/026605 (“'605 PCT”), entitled, “Multilayered Disposable Cartridge for Ferrofluid-Based Assays and Methods of Use” (see, e.g., paragraph [0019]-[0027], FIGS. 1-4) which is incorporated by reference herein in its entirety.

In some embodiments of the present disclosure, such cartridges are modified such that at least one (and in some present embodiments, at least two, and in some embodiments, all) fluidic channels include at least one set of a plurality of spatially distinct capture zones. In some embodiments, each zone is configured with at least one unique oligonucleotide- or PNA-probe complementary to the unique oligonucleotide- or PNA-probe used to make one particular unique label probe for (for example) a specific analyte (in some embodiments, for tracking at least one individual sample). Thus, each specific zone is custom-tailored to a specific target analyte.

Such a set/plurality of distinct capture zones can be arranged in parallel or serial, depending upon the embodiment. For example, in some embodiments, similar to the capture/analysis windows of the '605 PCT, such windows can correspond to a viewing port (either one large window for all capture zones, or individual windows for each single capture zone). Thus, in a serial arrangement, each zone is arranged one after another in a fluidic channel, thus, a fluid flow of such a fluidic channel encounters the first capture zone, then the second capture zone, and so on. In a parallel capture zone arrangement, a fluidic channel includes an area upon which each capture zone is arranged side-by-side; in such an arrangement, the fluid flow in such a fluidic channel encounters all captures zones at the same time.

Such cartridges, according to some embodiments, may also include, alternatively or in addition to the capture zones (seen in step 6, C1-C8 in FIG. 1 which depicts the capture zones for one lane, which can be repeated for one or more lanes, optionally for all lanes of the cartridge), a plurality of capture particles each configured with at least one unique oligonucleotide- or PNA-probe complementary to at least one particular unique label probe, e.g., of a specific analyte. Such capture particles may also include, in some embodiments, at least one unique fluorescent tag (e.g., corresponding to a respective at least one unique oligonucleotide- or PNA-probe), such that, the particles, when exposed to a fluorescing wavelength of light, will glow, i.e., emit light at a specific wavelength which can be imaged. A resulting image can be processed to determine a number of particles (which have been bound to the analyte and captured or flowed through a channel, for example). In some embodiments, the assay system and not the cartridge, provides a plurality of, and in some embodiments, all of the above note functionality.

Examples of cartridges, and assay systems which use such cartridges, according to some embodiments, are presented above, in the Summary section.

In some embodiments, any cartridge, lane, system discussed herein can correspond to a cartridge, lane, and/or system of Ancera LLC's PIPER cartridge/system.

FIG. 1 illustrates a schematic overview of a multi-sample analyte analysis method according to some embodiments of the present disclosure. Such a method can be carried out using, for example, a cartridge similar to that which is disclosed in the '605 PCT, and in some embodiments, a similar cartridge modified as noted above (e.g., one or more sets of a plurality of specific capture zones). Accordingly, samples S1-S8 (i.e., n #of samples), which, for example, can be cells (e.g., bacteria), are labeled. Specifically target analytes (TAs) in each of the individual samples are labeled with a unique label probe. Each of the unique label probes is attached to a specific binding agent of a respective TA. In some embodiments, the specific binding agent comprises at least one of an antibody, aptamer, and lectin configured to bind to a respective TA. The method, in some embodiments, can further include incubating each sample, and optionally washing and/or cross-linking each sample as discussed herein. In some embodiments, the plurality of individual (and processed—see above) samples are combined into a mixture. In some embodiments, the mixture is added to a ferrofluid. Thereafter, the mixture is flowed (e.g., in some embodiments, an aliquot is directed in one or more fluidic channels) through one or more fluidic channels of a cartridge (according to some embodiments, see above), such that, each flow is directed over one or more sets of a plurality of capture zones.

Upon the cartridge, and in particular, the fluidic channels being subjected to a particular magnetic field, the ferrofluid, as detailed in the '605 PCT, and other published patents and applications incorporated herein by reference, causes particles within the flow (e.g., bacteria, animal/plant cells, and the like) to separate into, for examples, one or more lines of particles based on size (for example). The at least one line of particles can be directed to a location in the fluidic channel or a portion in fluidic communication thereto—e.g., the capture zones. As one of skill in the art will appreciate, some embodiments of the present disclosure include a universal cartridge which can be prepared with capture probes coated on specific regions (e.g., capture regions/zones) of each channel and can be configured for a multiplex assay (e.g., 2-plex, 4-plex, 8-plex). In some embodiments, particles may be pushed up to a capture zone(s), without focusing the particles into lines of particles.

In some embodiments, such capture zones can be coated onto a capture layer of a universal cartridge using, for example, a mask to guide application of reagents. For example, the capture surface will be first coated with avidin, streptavidin, neutra-avidin, or modified versions of these. In some embodiments, the capture surface is coated via conventional methods known in the art and/or described herein. After washing and blocking of the surface to prevent non-specific binding of additional reagents, the capture areas will be incubated with individual biotinylated unique oligonucleotide- or PNA-probe capture probes and washed to remove unbound probe. These cartridges can be used for any assay that uses binders (such as antibodies) pre-labelled with corresponding complementary unique oligonucleotide- or PNA label probes. FIG. 1 provides an illustration for an 8-plex assay as a representative of the disclosure herein. Similarly, any other number of sample analyses are contemplated herein, including 1-plex, 2-plex, 3-plex, 4-plex, 5-plex, 6-plex, 7-plex, 8-plex, etc. Any other number of samples can be tested with a suitably designed probe set and coated cartridge.

As noted above, each capture zone may be configured to capture a specific target analyte, and testing/analysis for the specific target analyte can be (and is in some embodiments) specific to a particular sample of the mixture.

In some embodiments, the plurality of capture particles is flowed over the capture zones and binds with specific target analytes which are captured by such zones. The capture particles then bind with target analytes. Thereafter, the capture zones are subjected to a fluorescing wavelength of radiation/light, and imaged. The images produced can be analyzed to determine the number of glowing spots—each representing a specific, target analyte captured. Thus, the results for each sample, according to some embodiments, can be obtained directly from the capture zone corresponding to that sample.

For example, as shown in FIG. 1, eight (8) capture zone/probe areas (e.g., C1 through C8) can be the same for each fluidic channel, and each fluidic channel receives a separate mix of eight (8) samples that were preincubated with sample specific unique labeled analyte specific agent. For these assays, the combined samples are mixed with ferrofluid before the mixture is analyzed. After capture, fluorophore labeled, analyte specific probes can be used to label the captured analyte in each sample specific areas. The capture areas may then excite at a wavelength specific for the fluorophore used and the fluorescence emission is imaged and analyzed to provide area specific, original sample specific results. Additional target specific confirmation labelling—which, in some embodiments, corresponds to the use of the plurality of capture particles), is performed.

As noted in the Summary above, in some embodiments, one and/or another of (and in some embodiments, a plurality of, and in some embodiments, all of) the following features, functionality, structure, steps or clarifications, can be included, yielding yet further embodiments of the present disclosure:
the at least one of capturing, collecting, directing, and counting captured target analytes/particles includes imaging the captured target analytes/particles;
at least one of the target analytes (TAs) comprises a food pathogen;
the TAs are selected from the group consisting of bacteria, parasites, mold cells, mold spores, yeast, protein, and nucleic acid;
prior to labeling, the method includes the step of diluting one or more of the samples at least once, and in some embodiments, a plurality of times;
most probable number (MPN) analysis of captured TAs (in some embodiments, MPN functionality includes incubating (enriching) each diluted sample, where incubating can be performed prior to barcode labeling, washing, and combining the plurality of individual samples into the mixture);
the at least one set of the plurality of spatially distinct capture zones are arranged along at least one fluidic channel;
and
the at least one set of the plurality of spatially distinct capture zones comprises a plurality of sets, and in some embodiments:
   the plurality of sets are arranged along at least one fluidic channel, or
   each of the plurality of sets are arranged along a different fluidic channel of a plurality of fluidic channels.

In some embodiments, any type of sample can be used according to a desired detection (e.g., of a particular analyte), including, for example:
a protein;
DNA or RNA; and/or
specific cells/cell-types.

For example, in embodiments for analysis of food pathogens, such pathogens, in some embodiments, are enriched or concentrated to detectable levels (e.g., incubation). Such pathogens can be found in, for example:
any food product;
any matrix commonly tested during growth or processing of food;
any feces sample commonly tested during growth or processing;
any animal food source;
water samples;
probiotics; and/or
vaccine samples.

For example, in embodiments for human diagnostics, analysis of the following can be accomplished via embodiments of the present disclosure:
blood pathogens;
skin bacteria;
fecal monitoring;
all pathogen testing; and/or
disease monitoring, including protein targets, DNA/RNA targets (e.g., in blood), and disease cells.

Other assays can be performed for such things as vaccine efficacy, and serological sampling.

Capture/label probes. In some embodiments, synthetic, non-natural probe sets can be provided which can be configured to melt (i.e., dissociate) at temperatures significantly above the optimum hybridization temperature of target specific FISH (Fluorescence In-Situ Hybridization) probes. In some embodiments, a sequence of non-natural probes can be configured to have no sequence homology with naturally occurring DNA or RNA sequences. Accordingly, in many embodiments, any number of synthetic non-natural probe sets can be configured and synthesized, e.g.: standard DNA or RNA oligonucleotides, protein nucleic acids (PNAs), modified PNAs, LNAs, or any nucleotide analog (or any possible combination of the foregoing).

Probe sets, in some embodiments, is composed of one sequence called the “Capture probe” and its complementary sequence called the “Label probe”. The Capture probe(s) is, in some embodiments, is modified on one with a ligand of reactive group to enable attachment to a solid phase. The Label probe, in some embodiments, is labeled with a ligand or reactive group to enable attachment to a binder specific to the analyte-of-interest. Multiple unique Capture-Label probe sets can be configured and synthesized (e.g., C1, C2, C3, C4, C5, and the like; L1, L2, L3, L4, L5, and the like).

Other factors for some embodiments of the current disclosure, include sample enrichment time, which can range between 0-48 hours (for example), depending on the pathogen of interest and/or its abundance. Enrichments longer than 48 hours may be necessary for some slow growing pathogens (for example). One of skill in the art will appreciate that according to some embodiments, all samples need not to be derived from the same type of sample.

Process Samples. In some embodiments, methods used to process samples may be similar to those used to immune-label cells. Accordingly, aliquots of each mixture can be incubated with anti-pathogen antibodies labeled with Label probes (e.g., L1 to Ln). In some embodiments, after a suitable incubation time, the labelled cells can be washed and re-suspended in a suitable buffer for capture on the capture surface (C1 to Cn). In some embodiments, once the label antibodies are bound to the surface of the target pathogen, it may be necessary to cross-link either the antibodies to the surface of the pathogen, or, cross-link the Label antibodies to each other to maintain the antibody:pathogen complex.

With respect to one and/or another of the disclosed embodiments, to cross-link the label antibodies to the surface of the pathogen, standard homo-bifunctional or hetero-bifunctional cross-linking agents may be used. Alternatively, to cross-link bound antibodies to each other, the label antibodies can be labeled with an additional non-native poly-nucleotide that can hybridize to a long a non-natural complementary sequence, thus joining multiple antibodies and increasing the avidity of linked antibodies to the pathogen.

Accordingly, at least some embodiments, of the present disclosure provide advantages of (for example):
allowing simultaneous testing of multiple samples;
eliminate a need to return to an original sample to find the positive sample;
a single cartridge configured to perform multiple different assays (or similar assays);
and
low assay cost.

### Definitions

A “label” is a moiety that facilitates detection of a molecule. Common labels in the context of the present invention include fluorescent, luminescent, light-scattering, and/or colorimetric labels. Suitable labels include enzymes and fluorescent moieties, as well as radionuclides, substrates, cofactors, inhibitors, chemiluminescent moieties, magnetic particles, and the like. Many labels are commercially available and can be used in the context of the invention. The term “polynucleotide” (and the equivalent term “nucleic acid”) encompasses any physical string of monomer units that can be corresponded to a string of nucleotides, including a polymer of nucleotides (e.g., a typical DNA or RNA polymer), peptide nucleic acids (PNAs, including standard unmodified PNAs, chemically modified PNAs and chiral and achiral PNAs), modified oligonucleotides (e.g., oligonucleotides comprising nucleotides that are not typical to biological RNA or DNA, such as 2′-O-methylated oligonucleotides), and the like. The nucleotides of the polynucleotide can be deoxyribonucleotides, ribonucleotides or nucleotide analogs, can be natural or non-natural, and can be unsubstituted, unmodified, substituted or modified. The nucleotides can be linked by phosphodiester bonds, or by phosphorothioate linkages, methylphosphonate linkages, boranophosphate linkages, or the like. The polynucleotide can additionally comprise non-nucleotide elements such as labels, quenchers, blocking groups, or the like. The polynucleotide can be, e.g., single-stranded or double-stranded.

A “nucleic acid target” or “target nucleic acid” refers to a nucleic acid sequence, or a ribonucleic acid sequence, or optionally a region thereof, that is to be detected.

A “polynucleotide sequence” or “nucleotide sequence” is a polymer of nucleotides (an oligonucleotide, a DNA, a RNA, a nucleic acid, etc.) or a character string representing a nucleotide polymer, depending on context. From any specified polynucleotide sequence, either the given nucleic acid or the complementary polynucleotide sequence (e.g., the complementary nucleic acid) can be determined.

The term “gene” is used broadly to refer to any nucleic acid associated with a biological function. Genes typically include coding sequences and/or the regulatory sequences required for expression of such coding sequences. The term gene can apply to a specific genomic sequence, as well as to a cDNA or an mRNA encoded by that genomic sequence. Genes also include non-expressed nucleic acid segments that, for example, form recognition sequences for other proteins. Non-expressed regulatory sequences include promoters and enhancers, to which regulatory proteins such as transcription factors bind, resulting in transcription of adjacent or nearby sequences.

The term “complementary” refers to a polynucleotide that forms a stable duplex with its “complement,” e.g., under relevant assay conditions. Typically, two polynucleotide sequences that are complementary to each other have mismatches at less than about 20% of the bases, at less than about 10% of the bases, preferably at less than about 5% of the bases, and more preferably have no mismatches.

Two polynucleotides “hybridize” when they associate to form a stable duplex, e.g., under relevant assay conditions. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology-Hybridization with Nucleic Acid Probes, part I chapter 2, “Overview of principles of hybridization and the strategy of nucleic acid probe assays” (Elsevier, New York).

As used herein, “Fluorescent In-situ Hybridization” or “FISH” is a method of localizing and detecting DNA or RNA sequences in morphologically preserved tissue sections or cell preparations. The FISH assay typically employs specially constructed DNA probes, which are directly labeled with fluorescent dyes. Detection of nucleic acid analytes in biological samples (DNA or RNA) can be broadly categorized into two types of methods: “whole-sample” and “in-situ” detection. In the whole-sample detection method, the cells in the sample are lysed, which releases the molecules contained in the cells, including the nucleic acid analytes, into sample solution. Then the quantities of the nucleic acid analytes of the entire biological sample are measured in the solution. In the in-situ detection method, the nucleic acid analytes identified within the host cells and their quantities are measured at an individual cell level. The cells may be individual cells, or in tissue slices. While the methods, compositions, and systems of the instant invention are primarily described herein with reference to in-situ detection, many features of the invention can also be applied to whole-sample detection. The methods described herein detect sequences in-situ.

As used herein, the term ZipCode is commonly used in molecular biology to describe unique molecular labels that can be used to identify a sample or target. When ZipCode is used with Anti-ZipCode, they commonly describe two complementary probes that hybridize to each other. Elsewhere in this application, the terms label probe and complementary capture probe or just capture probe are used to discuss these probes. The term barcode is used to identify a ZipCode labeled antibody.

In one aspect, the signal is generated by a hybridization chain reaction (HCR), wherein DNA to a substrate can accomplish the roles of recognition and signal amplification without any external inputs. This is accomplished by the triggered self-assembly of DNA nanostructures in a novel process termed hybridization chain reaction (HCR). Additionally, combining a fluorescent DNA intercalating dye for signal readout can be used in any of the methods or kits discussed herein.

In one aspect, a method for detecting one or more analytes using a ferrofluid medium is provided. In some embodiments, the method comprises flowing a mix comprising a ferrofluid medium containing one or more target analytes through at least one microfluidic channel. In some embodiments, the target analyte is captured in a specific zone of the microfluidic channel using target specific binders (such as antibodies, aptamers, lectins, etc) for subsequent analysis. In some embodiments, the present disclosure is directed to a method for analyzing a target analyte using a one-step FISH assay within a ferrofluid medium. In some embodiments, the present disclosure is directed to targeting specific ribosomal RNA (rRNA) that are unique to a pathogen of interest. In one embodiment, the specific ribosomal RNA target is common among all inclusivity strains to be detected in the assay. In some embodiments, the probe does not bind to strains that the assay is meant to exclude.

### Ferrofluid-Based Microfluidic System

In some embodiments, the FISH methods described herein are conducted in a cartridge/system/platform (e.g. Ancera LLC's PIPER cartridge/system) as apart of a ferrofluid-based microfluidic system. In some embodiments, the FISH analysis described herein are conducted in a cartridge as a part of a ferrofluid-based microfluidic system. One ferro-fluid system and cartridge design are described in detail in US 2018/0029033 which is incorporated by reference herein in its entirety. The cartridge includes a sample reservoir to receive a mixture of a plurality of target particles and a ferrofluidic solution; a capture region formed on the cartridge; a fluidic channel to communicate the mixture between the sample reservoir and the capture region; a magnetic ferrofluidic solution positioned inside the fluidic channel, and at least one pneumatic valve to communicate a quantity of the mixture from the sample reservoir. The magnetic ferrofluidic solution is excitable in response to an externally applied electromagnetic field to affect the ferrofluidic solution in the mixture.

Conventional ferrofluid-based cellular and biological particle manipulation scheme rely on current-carrying electrodes on an industrial printed circuit board (PCB). The magnetic fields generated in such devices may be short-range and limited by electrode spacing on the PCB traces. In order to keep the design of the fluidic cartridge simple and low-cost, the excitation PCB resides outside the cartridge structure.

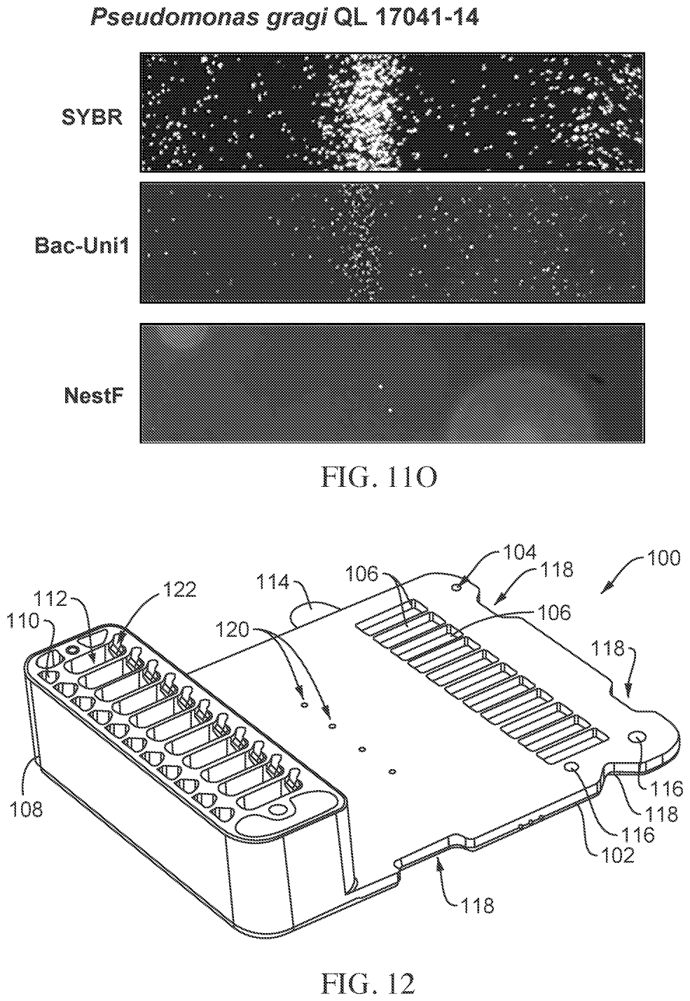

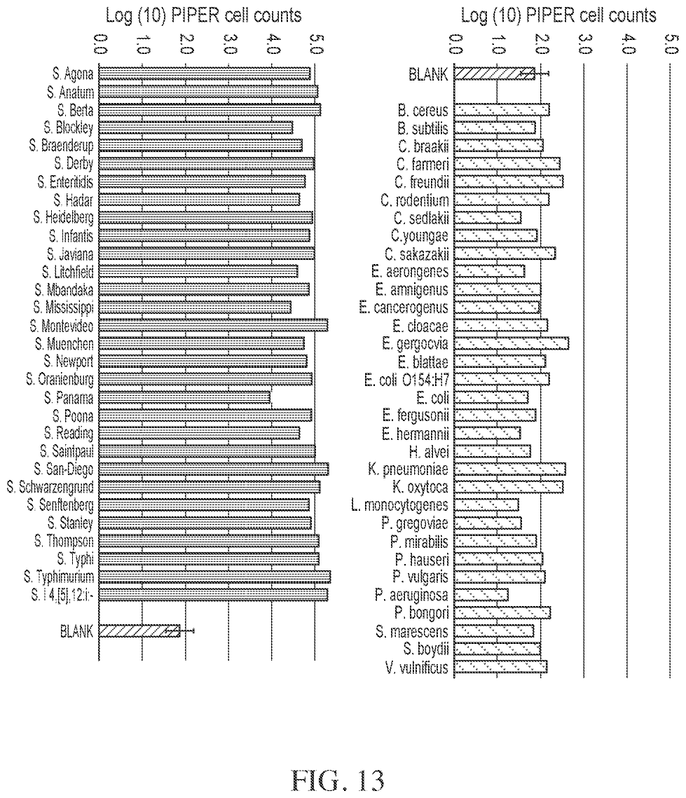

FIG. 12 is a perspective depiction of an exemplary embodiment of a cartridge that is configured to perform eleven independent, parallel assays. Other embodiments of a cartridge may be configured to perform a different number of parallel assays (such as 8 or 12), or they may be configured to run a single assay. The width of the cartridge 100 may change depending on the total number of assays supported. Cartridge 100 may comprise multiple layers integrated into a unitary or an integrated cartridge. In an alternative embodiment, cartridge 100 may comprise a single construction with various features discussed below integrated therein. Cartridge 100 includes multiple layers of thin plastic sheets and adhesives that define the fluid flow in the cartridge 102, cartridge-instrument alignment features 118 pump valves and a reservoir stack 108. Reservoir stack 108 may further include main reservoirs 112, return chimneys 122 and a secondary reagent reservoir 110 used to add reagents into each microfluidic channel. The cartridge may also comprise internal alignment features 104 and 116 that may be used to ensure proper registration between the internal layers during its construction. Note that all of these features (excluding 102) are part of an injection molded top layer. Other layers of the microfluidic device 102 are constructed and adhered to this molded layer.

Cartridge-instrument alignment features 118 enable aligning placement of cartridge 100 within an assay instrument (not shown). The alignment may ensure, in part, that the cartridge main channels can align directly (or approximately) over the electrodes of the excitation PCB. This may also ensure that any other interface to the cartridge (such as pneumatic input ports, 120, to control valves and pumping layers within the cartridge that are to pump fluid reagents within the cartridge) are aligned with the corresponding output from the instrument. Cartridge 100 may be inserted into an instrument slot (not shown) or may be placed at a designated space (such as a dedicated receptacle) within the assay instrument (not shown).

A plurality of cartridge analysis windows (or viewing ports) 106 may correspond with each of a plurality of microfluidic channels (not shown). As described below, the microfluidic channels (not shown) are formed in various layers of 102. Cartridge analysis windows 106 provide optical viewing ports to each of the capture zones contained in each microfluidic channel.

A reagent spotting mask 114 is used to facilitate the creation of capture zones in specific areas of a microfluidic channels during cartridge manufacture. This layer is removed prior to cartridge assembly. The mask may consist of a matrix of patterned openings cut into an adhesive or a soft gasket (e.g., silicone rubber, PDMS, etc.) that is temporarily affixed onto what becomes the upper surface of the main microfluidic channel. The assay reagents may thus be coated (or spotted) over that surface of the cartridge through the mask openings, prior to the assembly of the cartridge.

The internal alignment features 104 and 116 may optionally be used to assist in the assembly of the cartridge internal layers in order to ensure that each layer is properly aligned with and registered to its neighbors within a given positional tolerance. In some embodiments, the alignment features may be holes of a given shape (e.g., circular, square, hexagonal, diamond, etc.) that mate with alignment posts on an alignment jig.

In some embodiments, the cartridge may have pneumatic input ports 120. These ports may lead into pneumatic lines integrated into the cartridge. Together, they relay pressure and/or vacuum signals from the instrument to membrane valves (not shown) integrated into the body of the cartridge.

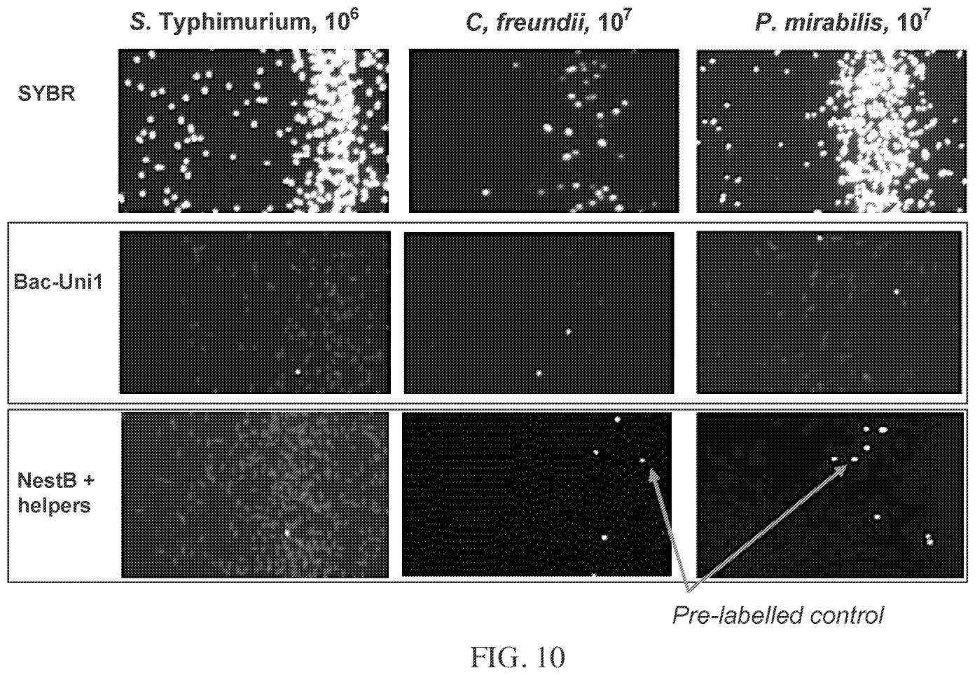

Reservoir stack 108, as described below, can retain the cartridge input fluids. For example, the reservoir stack 108 may receive and retain assay reagents which are then directed to the fluidic network (not shown in FIG. 10) of cartridge 100. Main reservoirs 112 typically receive ferrofluid and/or input sample reagents that are intended for the ferrofluidic assay. They may also be configured to receive additional reagents, as needed.

In some embodiments, reservoir stack 108 may support more than one set of reservoir wells per independent assay. Secondary reservoirs 110 may be configured to receive secondary reagents used for an assay under study. The secondary reagents may include labels, dyes, secondary antibodies, PCR reagents required for DNA amplification after cell capture, etc. In some implementations, the secondary reservoirs may be left blank or empty.

In some embodiments, specific areas of one layer of the microfluidic cartridge that align with windows 106 of FIG. 12 can be coated with specific analyte capture agents (ie: antibodies) to capture analytes to facilitate labeling and detection.

### Methods

In one aspect the present disclosure is directed to a method for performing a fluorescence in-situ hybridization assay using a ferrofluid-based microfluidic device comprising one or more of the following steps:
preparing a first mixture of ferrofluid and a sample containing or suspected of containing a target analyte, and adding this mixture into a main reservoir (112);
preparing a second mixture comprising a fluorescent labeled analyte specific probe designed to hybridize to a unique nucleotide sequence in the target analyte and other components and buffers required and add this reagent mixture into a secondary reservoir (110);
applying a magnetic field to flow the first mixture through a channel/lane comprising a capture region, wherein the capture region comprises one or more binding agents which capture the target analyte;
flowing the second solution into the channel;
allowing the probe to interact with the sample for a predetermined time;
applying a magnetic field to flow the ferrofluid through the channel to remove unbound probe;
counting the number of labeled cells.

In one aspect the present disclosure is directed to a method for performing a fluorescence in-situ hybridization assay using a ferrofluid-based microfluidic device comprising one or more of the following steps:
preparing a first mixture of ferrofluid and a sample containing or suspected of containing a target analyte, and adding this mixture into a main reservoir (112);
combining at least the first mixture and a second mixture comprising a labeled probe configured to hybridize to a unique nucleotide sequence in the target analyte and other components and buffers required and add this reagent mixture into a secondary reservoir (110);
flowing the combined mixture through a channel comprising a capture region while applying a magnetic field, wherein the capture region comprises one or more binding agents which capture the target analyte;
allowing the probe to interact with the sample for a predetermined time;
applying a magnetic field to flow the ferrofluid through the channel to remove unbound probe;
counting the number of labeled cells.

In one aspect the present disclosure is directed to a method for performing a fluorescence in-situ hybridization assay using a ferrofluid-based microfluidic device comprising one or more of the following steps:
preparing a first mixture of ferrofluid and a sample containing or suspected of containing a target analyte, and adding this mixture into a main reservoir (112);
obtaining a second mixture/solution comprising a fluorescent labeled analyte specific probe designed to hybridize to a unique nucleotide sequence in the target analyte and other components and buffers required and add this reagent mixture into a secondary reservoir (110);
applying a magnetic field to flow the first mixture through a channel comprising a capture region, wherein the capture region comprises one or more binding agents which capture the target analyte;
flowing the second mixture/solution into the channel;
allowing the probe to interact with the sample for a predetermined time;
applying a magnetic field to flow the ferrofluid through the channel to remove unbound probe;
counting the number of labeled cells.

### Probes

In some embodiments, the FISH method of the present disclosure targets specific ribosomal RNA (rRNA) that are unique to a target analyte of interest. In some embodiments, the target analyte is a pathogen. In some embodiments, the method of the present disclosure targets specific rRNA unique to the pathogen and is common among all inclusivity strains that need to be detected in the assay. In some embodiments, the selected probes do not bind to strains that the assay is meant to exclude.

In some embodiments, pathogen specific rRNA sequences described throughout the literature are contemplated for use with the FISH assay of the present disclosure. In some embodiments, pathogen specific single nucleotide polymorphisms (SNPs) are identified by searching rRNA sequence databases and such sequences are contemplated for the design of probes for the FISH assay of the present disclosure. These sequences may contain one or more SNP unique to the pathogen of interest. The exact location of the SNP or SNPs in the final probe sequence can be determined experimentally, or, based on prior probe design experience.

In some embodiments, the probes of the present disclosure are protein nucleic acid (PNA)-based FISH probes. In some embodiments, PNA-based FISH probes are 11 to 13 peptide nucleic acid bases long with the SNP sites approximately in the middle of the probe.

In some embodiments, protein nucleic acid (PNA) probes allow the use of short sequence specific probe designs and faster hybridization times. In some embodiments, the probes of the present disclosure are conventional oligo-FISH probes.

In some embodiments, FISH assay methods of the present disclosure are useful for the identification of gram-negative and gram-positive bacteria. In some embodiments, FISH assay methods of the present disclosure are useful for the identification of over 100 serovars of Salmonella sp. (Gram-negative) and several serovars of Listeria sp. (gram-positive).

In some embodiments, PNA-FISH assay methods of the present disclosure are useful for the identification of gram-negative and gram-positive bacteria. In some embodiments, PNA-FISH assay methods of the present disclosure are useful for the identification of over 100 serovars of Salmonella sp. (Gram-negative) and several serovars of Listeria sp. (gram-positive).

PNA probes can aggregate and keeping the probe monomeric can be difficult. To alleviate this, short un-labeled oligo probes are designed to bind specifically to the labeled FISH probes and the unlabeled helper probes. The oligo-probes are negatively charged and help keep the PNA probes in solution. The short oligo-probes dissociate from the PNA probes when the PNA probes bind to the target sequences and do not interfere in the assay.

Pathogen specific rRNA sequences are described throughout the literature and can be used with the methods of the present disclosure. Pathogen specific single nucleotide polymorphisms (SNPs) can also be identified by searching rRNA sequence databases. These sequences may contain one or more single nucleotide polymorphism unique to the pathogen of interest. The exact location of the SNP or SNPs in the final probe sequence can be determined without undue experimentation and based on prior probe design experience. Most commonly, PNA-based FISH probes, are 11 to 13 peptide nucleic acid bases long, with the SNP sites approximately in the middle of the probe.

The FISH probes are labeled with fluorescent dyes on their amino or carboxyl end. Dyes such as fluorescein, Alexa Fluor488, or any dye that is compatible with the specific detector can be used. A fluorescent-labeled FISH probe(s) can be used that targets a unique sequence on ribosomal RNA (rRNA) or other genetic component of the pathogen of interest. Typically, these target specific molecular probes can discriminate between sequences with as little as a single nucleotide mismatch. Multiple FISH probes (specific for separate unique SNPs) can be used to increase signal intensity.

### Hybridization

In some embodiments, the probe is allowed to hybridize with the target analyte. In some embodiments, the probe is allowed to hybridize with the target analyte for 1 min to 60 min.

In some embodiments, the hybridization (or, annealing) temperature is based on the target analyte. In some embodiments, the annealing temperature is based on the type of probe used. In some embodiments, the annealing temperature is based on the sequence used. In some embodiments, the annealing temperature is based on the use of one or more helper probes.

In some embodiments, the incubation temperature is based on the target analyte. In some embodiments, the incubation temperature is based on the type of probe used. In some embodiments, the incubation temperature is based on the sequence used.

In some embodiments, helper probes may be used. In some embodiments, helper probes are added to the hybridization solution to hybridize to the targeted DNA or RNA sequence close to the region targeted by the fluorescent target specific probe. These helper probes can make the specific target sequence more readily available for hybridization by the target specific probe.

In some embodiments, the incubation temperature is based on the use of one or more helper probes.

In some embodiments, the probe is allowed to hybridize/anneal with the target analyte at between 25° C. and 30° C. In some embodiments, the probe is allowed to hybridize/anneal with the target analyte at between 25° C. and 35° C. In some embodiments, the probe is allowed to hybridize/anneal with the target analyte at between 25° C. and 40° C. In some embodiments, the probe is allowed to hybridize/anneal with the target analyte at between 25° C. and 45° C. In some embodiments, the probe is allowed to hybridize/anneal with the target analyte at between 25° C. and 50° C. In some embodiments, the probe is allowed to hybridize/anneal with the target analyte at between 25° C. and 55° C. In some embodiments, the probe is allowed to hybridize/anneal with the target analyte at between 25° C. and 60° C. In some embodiments, the probe is allowed to hybridize/anneal with the target analyte at between 30° C. and 35° C. In some embodiments, the probe is allowed to hybridize/anneal with the target analyte at between 30° C. and 40° C. In some embodiments, the probe is allowed to hybridize/anneal with the target analyte at between 30° C. and 45° C. In some embodiments, the probe is allowed to hybridize/anneal with the target analyte at between 30° C. and 50° C. In some embodiments, the probe is allowed to hybridize/anneal with the target analyte at between 30° C. and 55° C. In some embodiments, the probe is allowed to hybridize/anneal with the target analyte at between 30° C. and 60° C. In some embodiments, the probe is allowed to hybridize/anneal with the target analyte at between 35° C. and 40° C. In some embodiments, the probe is allowed to hybridize/anneal with the target analyte at between 35° C. and 45° C. In some embodiments, the probe is allowed to hybridize/anneal with the target analyte at between 35° C. and 50° C. In some embodiments, the probe is allowed to hybridize/anneal with the target analyte at between 35° C. and 55° C. In some embodiments, the probe is allowed to hybridize/anneal with the target analyte at between 35° C. and 60° C. In some embodiments, the probe is allowed to hybridize/anneal with the target analyte at between 40° C. and 45° C. In some embodiments, the probe is allowed to hybridize/anneal with the target analyte at between 40° C. and 50° C. In some embodiments, the probe is allowed to hybridize/anneal with the target analyte at between 40° C. and 55° C. In some embodiments, the probe is allowed to hybridize/anneal with the target analyte at between 40° C. and 60° C. In some embodiments, the probe is allowed to hybridize/anneal with the target analyte at between 45° C. and 50° C. In some embodiments, the probe is allowed to hybridize/anneal with the target analyte at between 45° C. and 55° C. In some embodiments, the probe is allowed to hybridize/anneal with the target analyte at between 45° C. and 60° C. In some embodiments, the probe is allowed to hybridize/anneal with the target analyte at between 50° C. and 55° C. In some embodiments, the probe is allowed to hybridize/anneal with the target analyte at between 50° C. and 60° C. In some embodiments, the probe is allowed to hybridize/anneal with the target analyte at between 55° C. and 60° C.

In some embodiments, the probe is allowed to hybridize/anneal with the target analyte at 25° C. In some embodiments, the probe is allowed to hybridize/anneal with the target analyte at 30° C. In some embodiments, the probe is allowed to hybridize/anneal with the target analyte at 35° C. In some embodiments, the probe is allowed to hybridize/anneal with the target analyte at 40° C. In some embodiments, the probe is allowed to hybridize/anneal with the target analyte at 42° C. In some embodiments, the probe is allowed to hybridize/anneal with the target analyte at 45° C. In some embodiments, the probe is allowed to hybridize/anneal with the target analyte at 48° C. In some embodiments, the probe is allowed to hybridize/anneal with the target analyte at 50° C. In some embodiments, the probe is allowed to hybridize/anneal with the target analyte at 55° C. In some embodiments, the probe is allowed to hybridize/anneal with the target analyte at 60° C.

In some embodiments, the probe is allowed to hybridize/anneal with the target analyte for 1 min to 60 min at 25° C. In some embodiments, the probe is allowed to hybridize/anneal with the target analyte for 1 min to 60 min at 30° C. In some embodiments, the probe is allowed to hybridize/anneal with the target analyte for 1 min to 60 min at 35° C. In some embodiments, the probe is allowed to hybridize/anneal with the target analyte for 1 min to 60 min at 40° C. In some embodiments, the probe is allowed to hybridize/anneal with the target analyte for 1 min to 60 min at 42° C. In some embodiments, the probe is allowed to hybridize/anneal with the target analyte for 1 min to 60 min at 45° C. In some embodiments, the probe is allowed to hybridize/anneal with the target analyte for 1 min to 60 min at 50° C. In some embodiments, the probe is allowed to hybridize/anneal with the target analyte for 1 min to 60 min at 55° C. In some embodiments, the probe is allowed to hybridize/anneal with the target analyte for 1 min to 60 min at 60° C. In some embodiments, the probe is allowed to hybridize/anneal with the target analyte for about 5 minutes or less, about 10 minutes or less, about 15 minutes or less, about 20 minutes or less, about 25 minutes or less, about 30 minutes or less, about 35 minutes or less, about 40 minutes or less, about 45 minutes or less, about 50 minutes or less, about 55 minutes or less, or about 60 minutes or less.

In some embodiments, the probe is allowed to hybridize/anneal with the target analyte for about 5 minutes to about 60 minutes, from about 10 minutes to about 60 minutes, from about 15 minutes to about 60 minutes, from about 20 minutes to about 60 minutes, from about 25 minutes to about 60 minutes, from about 30 minutes to about 60 minutes, from about 35 minutes to about 60 minutes, from about 40 minutes to about 60 minutes, from about 45 minutes to about 60 minutes, from about 50 minutes to about 60 minutes, or from about 55 minutes to about 60 minutes.

In some embodiments, the probe is allowed to hybridize/anneal with the target analyte for about 5 minutes to about 55 minutes, from about 10 minutes to about 55 minutes, from about 15 minutes to about 55 minutes, from about 20 minutes to about 55 minutes, from about 25 minutes to about 55 minutes, from about 30 minutes to about 55 minutes, from about 35 minutes to about 55 minutes, from about 40 minutes to about 55 minutes, from about 45 minutes to about 55 minutes, or from about 50 minutes to about 55 minutes.

In some embodiments, the probe is allowed to hybridize/anneal with the target analyte for about 5 minutes to about 50 minutes, from about 10 minutes to about 50 minutes, from about 15 minutes to about 50 minutes, from about 20 minutes to about 50 minutes, from about 25 minutes to about 50 minutes, from about 30 minutes to about 50 minutes, from about 35 minutes to about 50 minutes, from about 40 minutes to about 50 minutes, or from about 45 minutes to about 50 minutes.

In some embodiments, the probe is allowed to hybridize/anneal with the target analyte for about 5 minutes to about 45 minutes, from about 10 minutes to about 45 minutes, from about 15 minutes to about 45 minutes, from about 20 minutes to about 45 minutes, from about 25 minutes to about 45 minutes, from about 30 minutes to about 45 minutes, from about 35 minutes to about 45 minutes, or from about 40 minutes to about 45 minutes.

In some embodiments, the probe is allowed to hybridize/anneal with the target analyte for about 5 minutes to about 40 minutes, from about 10 minutes to about 40 minutes, from about 15 minutes to about 40 minutes, from about 20 minutes to about 40 minutes, from about 25 minutes to about 40 minutes, from about 30 minutes to about 40 minutes, or from about 35 minutes to about 40 minutes.

In some embodiments, the probe is allowed to hybridize/anneal with the target analyte for about 5 minutes to about 35 minutes, from about 10 minutes to about 35 minutes, from about 15 minutes to about 35 minutes, from about 20 minutes to about 35 minutes, from about 25 minutes to about 35 minutes, or from about 30 minutes to about 35 minutes.

In some embodiments, the probe is allowed to hybridize/anneal with the target analyte for about 5 minutes to about 30 minutes, from about 10 minutes to about 30 minutes, from about 15 minutes to about 30 minutes, from about 20 minutes to about 30 minutes, or from about 25 minutes to about 30 minutes.

In some embodiments, the probe is allowed to hybridize/anneal with the target analyte for about 5 minutes to about 25 minutes, from about 10 minutes to about 25 minutes, from about 15 minutes to about 25 minutes, or from about 20 minutes to about 25 minutes.

In some embodiments, the probe is allowed to hybridize/anneal with the target analyte for about 5 minutes to about 20 minutes, from about 10 minutes to about 20 minutes, or from about 15 minutes to about 20 minutes.

In some embodiments, the probe is allowed to hybridize/anneal with the target analyte for about 5 minutes to about 15 minutes, or from about 10 minutes to about 15 minutes.

In some embodiments, the probe is allowed to hybridize/anneal with the target analyte for about 5 minutes to about 10 minutes.

### Labels

Labels are associated with the specific probes to allow them to emit signals that will be used in analysis. Any labels suitable for generating such signals can be used in the present invention. In some embodiments, the signals are generated by fluorophores. Fluorescent labeling, e.g., the process of covalently attaching a fluorophore to a probe that binds to a cellular constituent (such as a protein or nucleic acid) is generally accomplished using a reactive derivative of the fluorophore that selectively binds to a functional group contained in the target molecule. Common reactive groups include amine reactive isothiocyanate derivatives such as FITC and TRITC (derivatives of fluorescein and rhodamine), amine reactive succinimidyl esters such as NHS-fluorescein, and sulfhydryl reactive maleimide activated fluors such as fluorescein-5-maleimide.

In some embodiments, the FISH probes of the present disclosure are labeled with fluorescent dyes. When PNA probes are used, they are labeled on their amino or carboxyl end.

When standard oligoprobes are used, they must be synthesized with appropriate terminal nucleic acid bases to facilitate labeling. A wide variety of labels are well known in the art and can be adapted to the practice of the present disclosure. A number of fluorescent labels are well known in the art, including but not limited to phycoerythrin, rhodamine, Alexa Fluor 488, cyanine, and fluorescein, green fluorescent protein (GFP) and variants thereof (e.g., cyan fluorescent protein and yellow fluorescent protein), and quantum dots. Fluorophores emitting at various wavelengths (including tandem conjugates of fluorophores that can facilitate simultaneous excitation and detection of multiple labeled species) are contemplated for use in the methods as discussed herein. Fluorescent resonance energy transfer (FRET) methods are additionally contemplated herein in the design of fluorescent labeling of the rRNA or PNA probes of the present disclosure. Also, other labels such as non-fluorescent colorimetric dyes, luminescent labels, light-scattering labels (e.g., colloidal gold particles), and other labels have been described to label in-situ hybridization probes. If more sensitivity is required than that obtained with one fluorescent labeled target specific dye, multiple target specific FISH probes can be used if multiple unique sites are present in the target sequence. Other methods can be used to amplify the signal from one target molecule and are often used when the number of target sequences per cell is low. These signal amplification methods include HCR (hybridization chain reaction), branched DNA (bDNA), SABER, and other similar technologies, as well as target amplification approaches such as in-situ PCR.

Following a fluorescent labeling reaction used to label binders or probes, it is often necessary to remove any non-reacted fluorophore from the labeled target molecule. This is often accomplished by size exclusion chromatography, taking advantage of the size difference between fluorophore and labeled protein, nucleic acid, etc. Fluorophores may interact with the separation matrix and reduce the efficiency of separation. For this reason, specialized dye removal columns that account for the hydrophobic properties of fluorescent dyes are sometimes used. Reactive fluorescent dyes are available from many sources. They can be obtained with different reactive groups for attachment to various functional groups within the target molecule. They are also available in labeling kits that contain all the components to carry out a labeling reaction.

In some embodiments, labels of the present invention comprise one or more fluorescent dyes, including but not limited to fluorescein, rhodamine, Alexa Fluor dyes, DyLight fluors, ATTO Dyes, or any analogs or derivatives thereof.

In some embodiments, labels of the present invention include but are not limited to fluorescein and chemical derivatives of fluorescein; Eosin; Carboxyfluorescein; Fluorescein isothiocyanate (FITC); Fluorescein amidite (FAM); Erythrosine; Rose Bengal; Methylene blue; Laser dyes; Rhodamine dyes (e.g., Rhodamine, Rhodamine 6G, Rhodamine B, Rhodamine 123, Auramine O, Sulforhodamine 101, Sulforhodamine B, and Texas Red).

In some embodiments, labels of the present invention include but are not limited to ATTO dyes; Acridine dyes (e.g., Acridine orange, Acridine yellow); Alexa Fluor; 7-Amino actinomycin D; 8-Anilinonaphthalene-1-sulfonate; Auramine-rhodamine stain; Benzanthrone; 5,12-Bis(phenylethynyl)naphthacene; 9,10-Bis(phenylethynyl)anthracene; Blacklight paint; Brainbow; Calcein; Carboxyfluorescein; Carboxyfluorescein diacetate succinimidyl ester; Carboxyfluorescein succinimidyl ester; 1-Chloro-9,10-bis(phenylethynyl)anthracene; 2-Chloro-9,10-bis(phenylethynyl)anthracene; 2-Chloro-9,10-diphenylanthracene; Coumarin; Cyanine dyes (e.g., Cyanine such as Cy3 and Cy5, ZW800, ZW700, DiOC6, SYBR Green I); DAPI, Dark quencher, DyLight Fluor, Fluo-4, FluoProbes; Fluorone dyes (e.g., Calcein, Carboxyfluorescein, Carboxyfluorescein diacetate succinimidyl ester, Carboxyfluorescein succinimidyl ester, Eosin, Eosin B, Eosin Y, Erythrosine, Fluorescein, Fluorescein isothiocyanate, Fluorescein amidite, Indian yellow, Merbromin); Fluoro-Jade stain; Fura-2; Fura-2-acetoxymethyl ester; Green fluorescent protein, Hoechst stain, Indian yellow, Indo-1, Lucifer yellow, Luciferin, Merocyanine, Oxazin dyes (e.g., Cresyl violet, Nile blue, Nile red); Perylene; Phenanthridine dyes (Ethidium bromide and Propidium iodide); Phloxine, Phycobilin, Phycoerythrin, Phycoerythrobilin, Pyranine, Rhodamine, Rhodamine 123, Rhodamine 6G, RiboGreen, RoGFP, Rubrene, SYBR Green I, (E)-Stilbene, (Z)-Stilbene, Sulforhodamine 101, Sulforhodamine B, Synapto-pHluorin, Tetraphenyl butadiene, Tetrasodium tris(bathophenanthroline disulfonate) ruthenium (II), Texas Red, TSQ, Umbelliferone, or Yellow fluorescent protein.

In some embodiments, labels of the present invention include but are not limited to the Alexa Fluor family of fluorescent dyes (Molecular Probes, Oregon). Alexa Fluor dyes are typically used as cell and tissue labels in fluorescence microscopy and cell biology. The excitation and emission spectra of the Alexa Fluor series cover the visible spectrum and extends into the infrared. The individual members of the family are numbered according roughly to their excitation maxima (in nm). Alexa Fluor dyes are synthesized through sulfonation of coumarin, rhodamine, xanthene (such as fluorescein), and cyanine dyes. Sulfonation makes Alexa Fluor dyes negatively charged and hydrophilic. Alexa Fluor dyes are generally more stable, less prone to photobleaching, brighter, and less pH-sensitive than common dyes (e.g. fluorescein, rhodamine) of comparable excitation and emission, and to some extent the newer cyanine series. However, they are also more expensive. Exemplary Alexa Fluor dyes include but are not limited to Alexa Fluor-350, Alexa Fluor-405, Alexa Fluor-430, Alexa Fluor-488, Alexa Fluor-500, Alexa Fluor-514, Alexa Fluor-532, Alexa Fluor-546, Alexa Fluor-555, Alexa Fluor-568, Alexa Fluor-594, Alexa Fluor-610, Alexa Fluor-633, Alexa Fluor-635, Alexa Fluor-647, Alexa Fluor-660, Alexa Fluor-680, Alexa Fluor-700, or Alexa Fluor-750.

In some embodiments, labels of the present invention comprise one or more members of the DyLight Fluor family of fluorescent dyes (Dyomics and Thermo Fisher Scientific). Exemplary DyLight Fluor family dyes include but are not limited to DyLight-350, DyLight-405, DyLight-488, DyLight-549, DyLight-594, DyLight-633, DyLight-649, DyLight-680, DyLight-750, or DyLight-800.

In some embodiments, when pairs of dyes are used (as described in greater detail herein below) the activator choices include Alexa Fluor405, 488, 532 and 568, and the emitter choices include Cy5, Cy5.5, Cy7, and 7.5. Using these particular choices, because they can be mixed and matched to give functional dye pairs, there are 16 possible pairs (4×4) in all. In some embodiments, for RNA FISH, emitters used are Alexa Fluor 647 or Dynomics 632, Cy5.5, Cy7, and IR800CW. In some embodiments, for DNA FISH, they are Alexa Fluor647, Cy5.5, Alexa Fluor 750 and Alexa Fluor 790.

In some embodiments, the same type of labels can be attached to different probes for different types of cellular constituents, including nucleic acids and proteins.

For example, in some embodiments, DNA, RNA, or PNA probes are labeled with either Cy3 or Cy5 that has been synthesized to carry an N-hydroxysuccinimidyl ester (NHS-ester) reactive group. Since, NHS-esters react readily only with aliphatic amine groups, which nucleic acids lack, nucleotides have to be modified with aminoallyl groups. This can be done through incorporating aminoallyl-modified nucleotides during synthesis reactions. In some embodiments, labels are spaced to avoid quenching effects.

For example, in some embodiments, protein probes (e.g., antibodies) are also labeled with either Cy3 or Cy5. For protein labeling, Cy3 and Cy5 dyes sometimes bear maleimide reactive groups instead. The maleimide functionality allows conjugation of the fluorescent dye to the sulfhydryl group of cysteine residues. Cysteines can be added and removed from the protein domain of interest via PCR mutagenesis. Cy5 is sensitive to the electronic environment in which it resides. Changes in the conformation of the protein to which the label is attached can produce an enhancement or quenching of the emission. The rate of this change can be measured to determine enzyme kinetic parameters. Cy3 and Cy5 are used in proteomics experiments so that samples from two sources can be mixed and run together thorough the separation process. This eliminates variations due to differing experimental conditions that are inevitable if the samples were run separately. These variations make it extremely difficult, if not impossible, to use computers to automate the acquisition of the data after the separation is complete. Importantly, using these dyes makes the automation trivial.

In some embodiments, the methods herein further comprise a step of activating the one or more dyes embodied herein. In some embodiments, the label in the capture region are activated via methods known in the art that are dependent on the identify of the fluorescent dye.

One of skill in the art would readily appreciate that choices for a label are determined based on a variety of factors, including, for example, size, types of signals generated, methods of attachment to or incorporation into a probe, properties of the cellular constituents, including their locations within the cell, properties of the cells, types of interactions being analyzed, and so forth.

In some embodiments, labels such as fluorophores are attached to the probes as a secondary addition. In these embodiments, the probes are synthesized or formed prior to the addition of the labels. In some embodiments, labels such as fluorophores are attached to specific locale of the probes. For example, pre-synthesized probes (e.g., oligonucleotides or peptides) are mixed with fluorophores under predefined reaction conditions such that attachment of the fluorophores to the probes results.

In some embodiments, labels are embedded within the probes themselves. In these embodiments, one or more labels are incorporated into probes while they are being synthesized or formed. For example, a fluorophore can be embedded in an oligonucleotide probe during synthesis. In some embodiments, one or more labels (e.g., fluorophores) are attached to multiple identical probes (e.g., oligos with identical sequences).

In some embodiments, probes include multiple specific probes, which are differentially labeled (i.e., at least two of the chromosome-specific probes are differently labeled).

### Binders

In some embodiments, the ferrofluid-based microfluidic device for performing the FISH assay uses cartridges, with an example depicted in FIG. 12, which feature a capture/imaging zone where the target analyte is captured by one or more binders. In some embodiments, the cartridges are prepared with a pre-coated pathogen-specific capture/imaging zone.

In some embodiments, binders can be specific antibodies or antibody-like molecules, aptamers, lectins or other (pathogen-specific) molecules which recognize and bind the target analyte (pathogen) on the capture surface. In some embodiments, binder is attached to the capture surface though avidin/biotin chemistry. In some embodiments, binder is attached to the capture surface though click chemistry. In some embodiments, binder is attached to the capture surface though NHS-ester chemistry. In some embodiments, binder is attached to the capture surface though isothiocyanate chemistry. In some embodiments, binder is attached to the capture surface though carbodiimide chemistry. In some embodiments, binder is attached to the capture surface though chemistry as known in the art (e.g. chemical/passive, chemical surface modification methods). Samples containing the target analyte (pathogens) are then mixed with ferrofluid and added to the sample reservoir of a cartridge that has been inserted into the instrument. Each cartridge is designed to process 12 samples. After all samples are added the hybridization probes and reagents are added to the 12 dye reservoirs—one for each lane/sample of the cartridge. The instrument will then pump the samples through the cartridge, push-up and focus the pathogens on the capture surface where they are captured by the individual binders. After a set amount of time, the hybridization probes and reagents are pumped into the cartridge, displacing the ferrofluid from the capture zone. The temperature on the cartridge is then increased to the hybridization/annealing temperature for a set time. Then additional ferrofluid is pumped into the capture zone to displace the hybridization reagents and the cells are counted using fluorescence.

### Detection Reagent

In some embodiments, additional ingredients are added along with the fluorescent-labeled probe. Those additional ingredients include one or more helper probe, a permeabilization agent, a chelating agent, a pH controlling buffer, a osmolarity agent, and an autofluorescence quencher.

In some embodiments, the fluorescent in-situ hybridization methods herein comprise the use of helper probes in the hybridization reaction. Helper probes bind to the rRNA adjacent to, or close to the actual FISH probe binding site. They are unlabeled PNA or oligonucleotides that help dissociate the rRNA from ribosomal proteins and disrupt native secondary and tertiary structures in the vicinity of the target site which allows for more efficient hybridization of the FISH probe. The design of helper probes is known to those of ordinary skill in the art without undue experimentation.

In some embodiments, the fluorescent in-situ hybridization methods herein comprise the use of one or more permeabilization agents. In some embodiments, the fluorescent in-situ hybridization methods herein comprise the use of one or more permeabilization agents selected from anionic detergents (such as SDS) and membrane-active peptides (such as Polymyxin B, Colistin B).

In some embodiments, the fluorescent in-situ hybridization methods herein comprise the use of one or more chelating agents. In some embodiments, the fluorescent in-situ hybridization methods herein comprise the use of one or more chelating agents selected from ethylenediaminetetraacetic acid (EDTA), ethylene glycol-bis(O-aminoethyl ether)-N,N,N′,N′-tetraacetic acid (EGTA), iminodisuccinic acid (IDS), diethylenetriaminepentaacetic acid (DTPA), nitrilotriacetic acid (NTA), ethylenediaminedisuccinic acid (EDDS), methylglycinediacetic acid (MGDA), L-glutamic acid N,N-diacetic acid (GLDA), ethylenediamine-N,N′-diglutaric acid (EDDG), ethylenediamine-N,N′-dimalonic acid (EDDM), 3-hydroxy-2,2-iminodisuccinic acid (HIDS), 2-hydroxyethyliminodiacetic acid (HEIDA), and pyridine-2,6-dicarboxylic acid (PDA). In some embodiments, the chelator is EDTA.

In some embodiments, the fluorescent in-situ hybridization methods herein comprise the use of one or more buffers. In some embodiments, the fluorescent in-situ hybridization methods herein comprise the use of a buffer to control pH. In some embodiments, the fluorescent in-situ hybridization methods herein comprise the use of Tris-HCl buffer to control pH. In some embodiments, the fluorescent in-situ hybridization methods herein are buffered at a pH of 6.9-8.9. In some embodiments, the fluorescent in-situ hybridization methods herein are buffered at a pH of 7-9. In some embodiments, the fluorescent in-situ hybridization methods herein are buffered at a pH around 7. In some embodiments, the fluorescent in-situ hybridization methods herein are buffered at a pH around 8. In some embodiments, the fluorescent in-situ hybridization methods herein are buffered at a pH around 9.

In some embodiments, the fluorescent in-situ hybridization methods herein comprise the use of one or more osmolarity agents. In some embodiments, the fluorescent in-situ hybridization methods herein comprise the use of one or more osmolarity agents selected from magnesium chloride, magnesium sulfate, lithium chloride, sodium chloride, potassium chloride, lithium sulfate, sodium sulfate, potassium sulfate, sodium hydrogen phosphate, potassium hydrogen phosphate.

In some embodiments, the fluorescent in-situ hybridization methods herein comprise the use of one or more autofluorescence quenchers. In some embodiments, the fluorescent in-situ hybridization methods herein comprise the use of one or more autofluorescence quenchers selected from TrueVIEW, TrueBlack® and Sudan Black.

In some embodiments, the number of ribosomes in a target analyte cell may be low when the cells are not actively dividing. In some embodiments, glucose, pyruvate or a mixture of glucose and pyruvate can be added to cells and incubated to stimulate their metabolic activity and increase the level of ribosomes in those cells. In some embodiments, cells in a low metabolic state, such as those from an enriched sample where the cells may be in a stationary or late stationary growth phase can be treated with glucose, pyruvate or a mixture of glucose pyruvate to stimulate their metabolic activity and increase the level of ribosomes in those cells. In some embodiments, cells present in samples may be damaged and may have an extended lag-phase before exponential growth may also benefit from treatment with glucose, pyruvate or a mixture of glucose and pyruvate to boost their metabolic activity and increase their levels of ribosomes. In some embodiments, samples can be treated with an equal volume of phosphate buffered saline containing 200 mM glucose and 4% pyruvate at 37° C. for 30 minutes.

### Applications

In some embodiments, the ferrofluid-based FISH assay is used to identify and/or enumerate any target analyte. In some embodiments, the ferrofluid-based FISH assay is used to identify and/or enumerate any cell type. In some embodiments, the ferrofluid-based FISH assay is used to identify and/or enumerate a wide range of microbial pathogens common in food industry. In some embodiments, the ferrofluid-based FISH assay is used to identify and/or enumerate a wide range of microbial pathogens common in food industry, while excluding other bacteria percent in the sample.

In some embodiments, pathogens are detected directly in aliquots of environmental, poultry or fecal samples without off-instrument sample processing. In some embodiments, since pathogens are detected directly, the need for additional equipment and personnel training is eliminated.

In some embodiments, pathogens can be detected directly in aliquots of enriched environmental, poultry or fecal samples without off-instrument sample processing, eliminating the need for additional equipment and personnel training.

In some embodiments, the pathogen is enriched before testing according to the methods discussed herein. In some embodiments, pathogen is not enriched before testing according to the methods discussed herein. In some embodiments, where the level of pathogen contamination is high, samples are collected and tested without enrichment.

In some embodiments, the ferrofluid-based FISH assay is used to identify and/or enumerate the presence of a bacteria. In some embodiments, the bacteria is selected from Mycobacterium, Streptococcus, Staphylococcus, Shigella, Campylobacter, Salmonella, Clostridium, Corynebacterium, Pseudomonas, Neisseria, Listeria, Vibrio, Bordetella, E. coli (including pathogenic E. coli, Pseudomonas aeruginosa, Enterobacter cloacae, Mycobacterium tuberculosis, Staphylococcus aureus, Helicobacter pylori, and Legionella. In some embodiments, the ferrofluid-based FISH assay is used to identify and/or enumerate Gram-positive and Gram-negative bacteria. In some embodiments, the ferrofluid-based FISH assay is used to identify and/or enumerate Gram-positive bacteria. In some embodiments, the ferrofluid-based FISH assay is used to identify and/or enumerate Gram-negative bacteria. In some embodiments, the ferrofluid-based FISH assay is used to identify and/or enumerate over 100 serovars of Salmonella sp. (Gram-negative) and several serovars of Listeria sp. (gram-positive). In some embodiments the methods herein identify and/or enumerate one or more of Citrobacter sedlakii, Proteus mirabilis, Klebsiella pneumoniae, Citrobacter freundii, E. coli, Proteus mirabilis, Salmonella Typhimurium, Salmonella Arizonae, Salmonella Newport, Salmonella Muenchen, Salmonella freundii, E. coli, Proteus mirabilis, Klebsiella pneumoniae, Enterobacter cancerogenus, Shingela sonnei, Staphylococous saprophyticus, Pseudomonas gragi.

In some embodiments, the ferrofluid-based FISH assay is not limited to the identification and enumeration of bacteria. In some embodiments, the ferrofluid-based FISH assay is used for the identification and enumeration of one or more viruses. In some embodiments, the ferrofluid-based FISH assay is used for the identification and enumeration of a virus selected from SARS-CoV-2, Infectious Haematopoietic Necrosis Virus, Poliovirus, Rabies Virus Parvovirus, Hepatitis A virus, Hepatitis B virus, Hepatitis C virus, Zika virus, Measles virus, Mumps virus, Rubella virus, HIV, Influenza virus, Rhinovirus, Rotavirus A, Rotavirus B, Rotavirus C, Respiratory Syncytial Virus (RSV), Varicella zoster, Norovirus, Denge Virus, Herpes Simplex Virus, Newcastle Disease Virus, coronaviruses, SARS, MERS, and White Spot Syndrome Virus.

In some embodiments, the ferrofluid-based FISH assay is used for the identification and enumeration of one or more fungi. In some embodiments, the ferrofluid-based FISH assay is used for the identification and enumeration of one or more fungi selected from Aspergillus, Candida, Blastomyces, Coccidioides, Cryptococcus, and Histoplasma.

In some embodiments, the ferrofluid-based FISH assay is used for the identification and enumeration of one or more parasites. In some embodiments, the ferrofluid-based FISH assay is used for the identification and enumeration of one or more parasites selected from Plasmodium (i.e. P. falciparum, P. malariae, P. ovale and P. vivax), Trypanosoma, Toxoplasma, Giardia, and Leishmania, Cryptosporidium, helminthic parasites: Trichuris spp. (whipworms), Enterobius spp. (pinworms), Ascaris spp. (roundworms), Ancylostoma spp. and Necatro spp. (hookworms), Strongyloides spp. (threadworms), Dracunculus spp. (Guinea worms), Onchocerca spp. and Wuchereria spp. (filarial worms), Taenia spp., Echinococcus spp., and Diphyllobothrium spp. (human and animal cestodes), Fasciola spp. (liver flukes) and Schistosoma spp. (blood flukes).

### Methods

### One-Step FISH Methodology

In some embodiments, the fluorescence in-situ hybridization detection of an analyte of the present disclosure involves preparing a mixture of bacterial culture with a polyethylene glycol stabilized ferrofluid (PEG ferrofluid, Ferrotec). In some embodiments this mixture is 1:1. In some embodiments, various reagent solutions are prepared and are added to one or more reagent reservoir of a cartridge.

In some embodiments, the detection reagent is added to a first reagent reservoir. In some embodiments, the detection reagent is a specific labeling solution which contains a Salmonella specific probe (NestB probe), helper probe 1, and helper probe 2 in a labeling buffer (Tris-HCl, EDTA, NaCl and SDS).

In some embodiments, the detection reagent is a non-specific labeling solution which contains Bac-Uni1 probe (which labels all bacteria) in the same labeling buffer (Tris-HCl, EDTA, NaCl and SDS). Labeling with Bac-Uni1 allows the method to assess the specificity of NestB. In some embodiments, the non-specific labeling solution is added to a second reagent reservoir.

In some embodiments, a control is used, as a control for the FISH assays. In some embodiments, SYBR-Green is used. In some embodiments, a 10,000× concentrated solution of SYBR-Green (Thermo Fisher) was diluted 100 times in growth media (BPW) immediately before use. In some embodiments, the SYBR-Green control is added to a third reservoir.

In some embodiments, this bacterial culture and PEG ferrofluid mixture is added to a sample reservoir of a cartridge.

In some embodiments, aliquots of the sample (bacterial cultures mixed with PEG ferrofluid) are loaded into the assigned sample reservoir on each of the three cartridges. In some embodiments, aliquots of 500 μL of the sample (bacterial cultures mixed with PEG ferrofluid) are loaded into the assigned sample reservoir on each of the three cartridges.

In some embodiments, each sample is analyzed with one of three different labeling reagents: “specific”, “non-specific” and SYBR-green. In some embodiments, aliquots of each of the labeling reagents are loaded in reagent reservoirs on the corresponding cartridge. In some embodiments, aliquots of 100 μL of each of the labeling reagents are loaded in reagent reservoirs on the corresponding cartridge.

In some embodiments, the labeling conditions for molecular probes (specific and non-specific) has an initial reagent flow used to displace the ferrofluid and introduce the labeling solution into the region of the lane containing the captured cells. In some embodiments, the initial reagent flow is for 6 min 15 sec at 10 μL/min. In some embodiments, the initial reagent flow is for 6 min 15 sec at 10 μL/min at 48° C. In some embodiments, the initial reagent flow is for 6 min 15 sec at 10 μL/min at 48° C. followed by a no flow incubation. In some embodiments, the initial reagent flow is for 6 min 15 sec at 10 μL/min at 48° C. followed by a no flow incubation at 48° C. In some embodiments, the initial reagent flow is for 6 min 15 see at 10 μL/min at 48° C. followed by a no flow incubation at 48° C. and a 25 min no-flow incubation at 42° C. In some embodiments, if other cartridge depths are used, the flow times and/or flow rates are adjusted to ensure that hybridization solution completely washes out any prior ferrofluid or buffer that was present in the cell capture zone.

In some embodiments, fluorescence images are measured at between 1 millisecond (ms) to 1000 ms exposure. In some embodiments, fluorescence images are measured at between 100 ms and 600 ms exposure.

In some embodiments, the labeling conditions for SYBR Green include a one-step incubation at 39° C. for 5 minutes. In some embodiments, SYBR Green fluorescence images are measured at between 100 ms and 200 ms exposure. In some embodiments, SYBR Green fluorescence images are measured at 100 ms exposure.

While various inventive embodiments have been described and illustrated herein, those of ordinary skill in the art will readily envision a variety of other means, functionality, steps, and/or structures (including software code) for performing the functionality disclosed and/or obtaining the results and/or one or more of the advantages described herein, and each of such variations and/or modifications is deemed to be within the scope of the inventive embodiments described herein. More generally, those skilled in the art will readily appreciate that all parameters, and configurations described herein are meant to be exemplary and that the actual parameters, and configurations will depend upon the specific application or applications for which the inventive teachings is/are used. Those skilled in the art will recognize, or be able to ascertain using no more than routine experimentation, many equivalents to the specific inventive embodiments described herein. It is therefore to be understood that the foregoing embodiments are presented by way of example only and that, within the scope of any claims supported by this disclosure and equivalents thereto, inventive embodiments may be practiced otherwise than as specifically described and claimed. Inventive embodiments of the present disclosure are also directed to each individual feature, system, apparatus, device, step, code, functionality and/or method described herein. In addition, any combination of two or more such features, systems, apparatuses, devices, steps, code, functionalities, and/or methods, if such features, systems, apparatuses, devices, steps, code, functionalities, and/or methods are not mutually inconsistent, is included within the inventive scope of the present disclosure. Further embodiments may be patentable over prior art by specifically lacking one or more features/functionality/steps (i.e., claims directed to such embodiments may include one or more negative limitations to distinguish such claims from prior art).

The above-described embodiments of the present disclosure can be implemented in any of numerous ways. For example, some embodiments may be implemented (e.g., as noted) using hardware, software or a combination thereof. When any aspect of an embodiment is implemented at least in part in software, the software code can be executed on any suitable processor or collection of processors, servers, and the like, whether provided in a single computer or distributed among multiple computers.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The terms “can” and “may” are used interchangeably in the present disclosure, and indicate that the referred to element, component, structure, function, functionality, objective, advantage, operation, step, process, apparatus, system, device, result, or clarification, has the ability to be used, included, or produced, or otherwise stand for the proposition indicated in the statement for which the term is used (or referred to).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. Accordingly, the following examples are offered to illustrate, but not to limit, the claimed invention.

### Example #1: Design and Experimental Setup for Multiplex Experiment

### General Methods

Oligo Design and Conjugation: ZipCode oligos each having a unique sequence (Gerry et al. 1999. J Mol. Biol. 292: 251-262) were designed (Table 1). Each ZipCode oligo contained a 5′-linked biotin attached to a 15-atom mixed polarity triethylene glycol spacer (5BioTEG) and an 18-atom hexa-ethylene glycol spacer (iSP18) to facilitate immobilization on a streptavidin-coated surface. Complementary AntiZipCode oligos were also designed (Gerry et al. 1999. J Mol. Biol. 292: 251-262; Table 1) which contained a 5′ amino modifier and 6-carbon spacer arm (5AmMC6) and an 18-atom hexa-ethylene glycol spacer (iSP18) to facilitate conjugation to an antibody. The oligonucleotides were synthesized by Integrated DNA Technologies. Each of the AntiZipCode oligos was separately conjugated to an anti-Salmonella goat polyclonal antibody via maleimide thiol chemistry using the Perkit Antibody-Oligo Conjugation kit (Cell Mosaic). The final oligo-antibody conjugates were de-salted and buffer exchanged with 1× PBS.

TABLE 1
ZipCode and AntiZipCode oligo sequences

| Oligo Name | Oligo Sequence |
|---|---|
| ZipCode-1 | 5′-/5BioTEG/iSp18/TGCGACCTCAGCATCGACCTCAGC-3′ |
| ZipCode-5 | 5′-/5BioTEG/iSp18/GACCACCTTGCGATCGGGTACAGC-3′ |
| ZipCode-13 | 5′-/5BioTEG/iSp18/CAGCGGTAGACCACCTATCGTGCG-3′ |
| ZipCode-25 | 5′-/5BioTEG/iSp18/GACCATCGTGCGGGTAGGTAGACC-3′ |
| AntiZipCode- | 5′-/5AmMC6/iSp18/GCTGAGGTCGATGCTGAGGTCGCA-3′ |
| 1 | |
| AntiZipCode- | 5′-/5AmMC6/iSp18/GCTGTACCCGATCGCAAGGTGGTC-3′ |
| 5 | |
| AntiZipCode- | 5′-/5AmMC6/iSp18/CGCACGATAGGTGGTCTACCGCTG-3′ |
| 13 | |
| AntiZipCode- | 5′-/5AmMC6/iSp18/GGTCTACCTACCCGCACGATGGTC-3′ |
| 25 | |

Cartridge Coating: The biotinylated ZipCode oligos were arrayed at discrete locations in a cartridge pre-coated with streptavidin in a humidity chamber overnight. The streptavidin-coated regions were blocked with PBS containing 0.1% casein and 1% PEG for 1 hour in a humidity chamber followed by 3 washes with TE (Tris-EDTA) buffer. To each coated region, 4 μL of a 5 nM solution of one of the 4 biotinylated ZipCode oligos in TE, so that each lane of the cartridge will contain a series of 4 windows with different capture oligos. The oligos were incubated for 30 minutes at room temperature in a humidity chamber. The liquid was aspirated from each window by changing tips between capture windows to avoid cross-contamination. Free oligo was removed by three additional TE washes. StabilCoat (SurModics) was subsequently added to each window and aspirated after 5 minutes. The cartridge was allowed to dry for 30 minutes in a fume hood. The mask was removed, and the cartridge was assembled and closed using a Carver press at 5,000 PSI for 30 seconds.

Immunolabeling: Cell suspensions were prepared in 250-1000 μL of PBS. To each sample, 25 μL of a different AntiZipCode-conjugated antibody (10 ng/mL) was added. The samples were mixed by vortexing for 3 seconds and incubated for 10 minutes at room temperature to allow the antibody to bind to the cells. The cells were then pelleted by centrifugation at 18,000 g for 8 minutes to remove free (unbound) antibody-oligo conjugate. The supernatant was carefully removed and discarded. The pellet was resuspended in 1 mL of PBST and vortexed for 3 seconds. The centrifugation and wash step was repeated once. After the final wash, the pelleted cells were re-suspended in 100 μL of 100 mM Tris-C1, pH 7.9 (for final volume of 400 μL once 4 samples are combined).

Pooling and Processing: All 4 re-suspended samples were combined for a final volume of 400 μL. To the pooled sample, 135 μL of ferrofluid was added. The pooled sample was loaded into a single lane of the cartridge and flowed for 20 minutes at 50° C. in the presence of the magnetic PCB to enable the AntiZipCode oligos on the antibodies to anneal to the appropriate ZipCode oligo on the cartridge. Each individual sample was localized to its known ZipCode oligo location and that location could then be scored for the presence or absence of bacteria in the sample.

Sample Labeling and Detection: For detection, the captured cells were either labeled with BacLite-Green or using Ancera's PNA FISH labeling protocol. For FISH labeling, the following reagents were combined in a 1.7 mL tube in the indicated order (See Table 2 for sequences of probes for FISH):
1040 μL of Base Buffer (350 mM EDTA, 75 mM Tris-C1, pH 8)
100.75 μL of MB grade water
13 μL of PNA Probe H1 (100 μM in 50% DMF, 100 mM Tris-C1, pH 8)
13 μL of PNA Probe H2 (100 μM in 50% DMF, 100 mM Tris-C1, pH 8)
3.25 μL of PNA Probe F (200 μM in 50% DMF, 100 mM Tris-C1, pH 8)
130 μL of 0.2% SDS solution

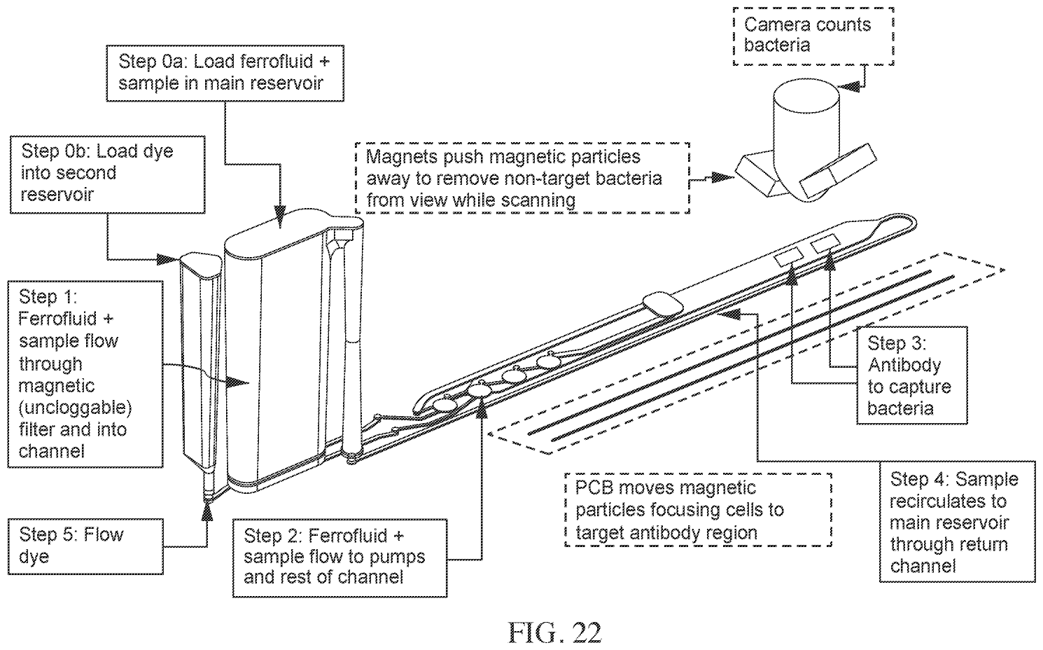

The solution was mixed by inverting the tube 10 times. 95 μL was loaded in the detection reagent reservoir (see, e.g. FIGS. 12 and 22). FISH hybridization was carried out by a 5-minute incubation at 48° C. followed by a 10-minute incubation at 42° C. Images were processed with proprietary software which enables enumeration of cells in each zone.

TABLE 2
Salmonella FISH probe and helper probe sequences

| PNA Name | Sequence |
|---|---|
| Probe H1 | GTTTTAACCTTGCGG |
| Probe H2 | CGCGTTAGCTCC |
| Probe F | GTCTACTTAAC-Lys-Alexa488 |

### Example #2: Various Cell Barcoding Assays

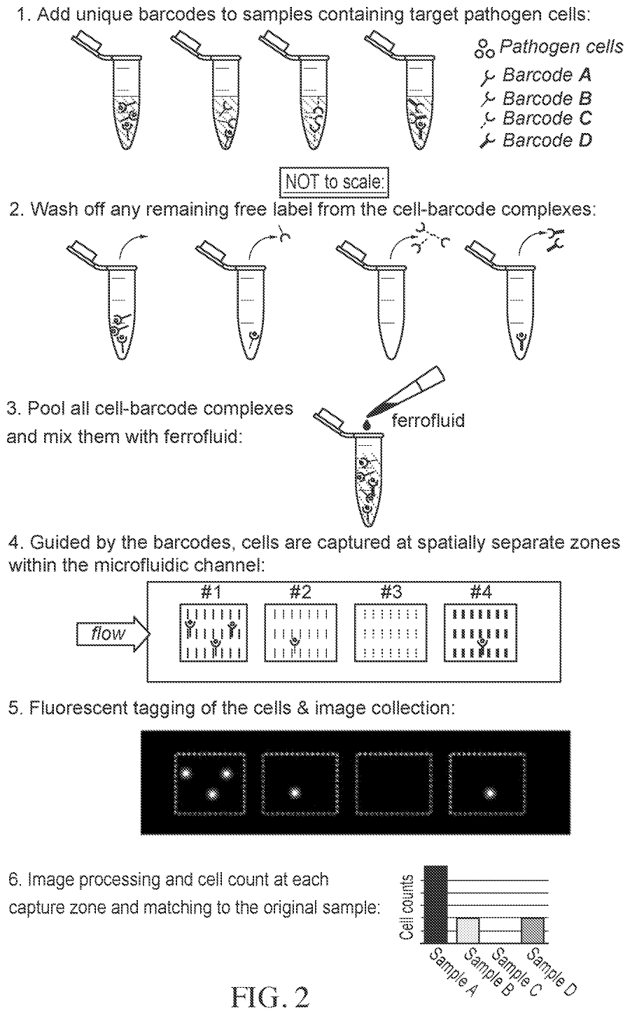

The assay is comprised of six principal steps depicted in FIG. 2. This example describes the preparation and processing of 4 individual samples that are processed in one lane of a 4-plex cartridge. The 4-plex cartridge has been prepared with 4 unique capture zones per lane. Each lane would receive a different set of 4 mixed samples processed in the same way as described here. The term ZipCode is a commonly used term in molecular biology used to describe unique molecular labels that can be used to identify a sample or target. When ZipCode is used with Anti-ZipCode, they commonly describe two complementary probes that hybridize to each other. Elsewhere in this application, the terms label probe and complementary capture probe or just capture probe are used to discuss these probes. The term barcode is used to identify a ZipCode labeled antibody.

Cell-barcode complex formation: An aliquot of ZipCode probe labeled antibody is added to each sample to be tested for the presence of the pathogen. Sample A receives barcode A, sample B receives barcode B, and so on.

Wash: Unbound ZipCode probe is then removed from labeled cells by centrifugation. After 2 washes, the cell pellet is resuspended in a small volume of buffer.

Sample pooling: 4 samples, each processed with a different ZipCoded antibody are then combined together and mixed with an aliquot of ferrofluid.

Cell separation: The pooled mixtures are then processed through a cartridge/system where cells are delivered to the array of capture zones. When sample flows through the capture zones, capture oligos hybridize with their complimentary barcodes, resulting in labelled cell being pulled from the solution and retained within the specific area on the surface of the microfluidic channel; cells from sample A end up in capture zone #1 that carries an oligo complimentary to the barcode A; cells from sample B end up in capture zone #2 that carries an oligo complimentary to the barcode B, and so on.

Fluorescent cell tagging & image collection: All cells are then labeled with a pathogen specific labeling method. The method could be a FISH assay described elsewhere in this application or using other pathogen specific reagents, such as labeled antibodies.

Cell counts: The cells captured and detected in each zone are counted. The results from each zone are directly tied to each sample and no further retesting of samples is required.

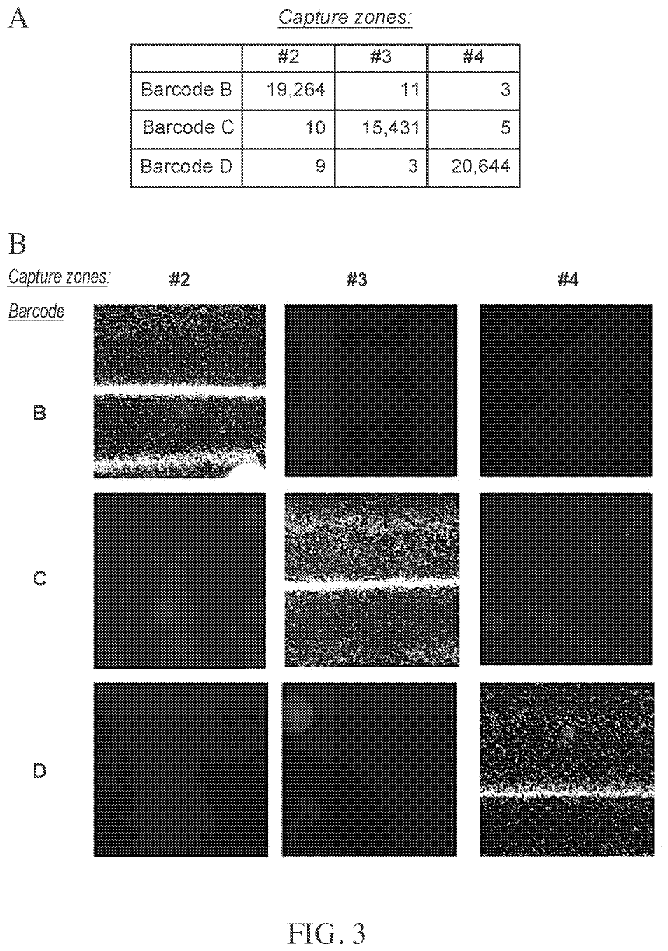

FIG. 3 shows the results obtained when the Barcode assay was tested using pre-stained cells. Salmonella cells (100 μl of a cell suspension) transferred into to a 1.7 ml tube and pelleted by centrifugation at 16,000 g for 3 minutes. The supernatant was removed, and the pellet was resuspended in 70 μl of BacLight Green Bacterial stain (ThermoFisher) reconstituted in DMSO as per manufacturer's instructions. After 24 hours incubation at 37° C. for with shaking, 990 μl of 0.05% Triton-X-100 in PBS was added to the cell suspension. Cells were pelleted by centrifugation at 16,000 g for 3 minutes, the supernatant was removed, and the pellet was resuspended in 50 μl of 0.05% Triton-X-100 in PBS. To wash the cells, the cells suspension was transferred into the new tube containing 950 μl of 0.05% Triton-X-100 in PBS and vortexed for 10 seconds. For the final wash, cells were pelleted by centrifugation as described above and resuspended in 1 mL of 0.05% Triton-X-100 in PBS buffer. After pelleting the cells by centrifugation as described above, cells were resuspended in 1 mL of PBS and counted using hemocytometer. Approximately 500,000 cells/sample were used for barcoded capture. FIG. 3A contains cell counts of simplified 3-sample 3-window experiment. Aliquots of pre-labelled cells were independently incubated with Barcode B (anti-ZipCode—antibody #5), Barcode C (anti-ZipCode—conjugated antibody #13), or Barcode D (anti-ZipCode—conjugated antibody #25). After washing and recovery of the barcoded cells, each complex was run in a separate lane containing various capture oligos #2 (zip oligo 5), #3 (zip oligo 13) or #4 (zip oligo 25) as indicated by the numbers above the table. Section B shows a small segment of each capture zone, magnified to reveal fluorescent cells. These results demonstrate the specificity of the barcode capture.

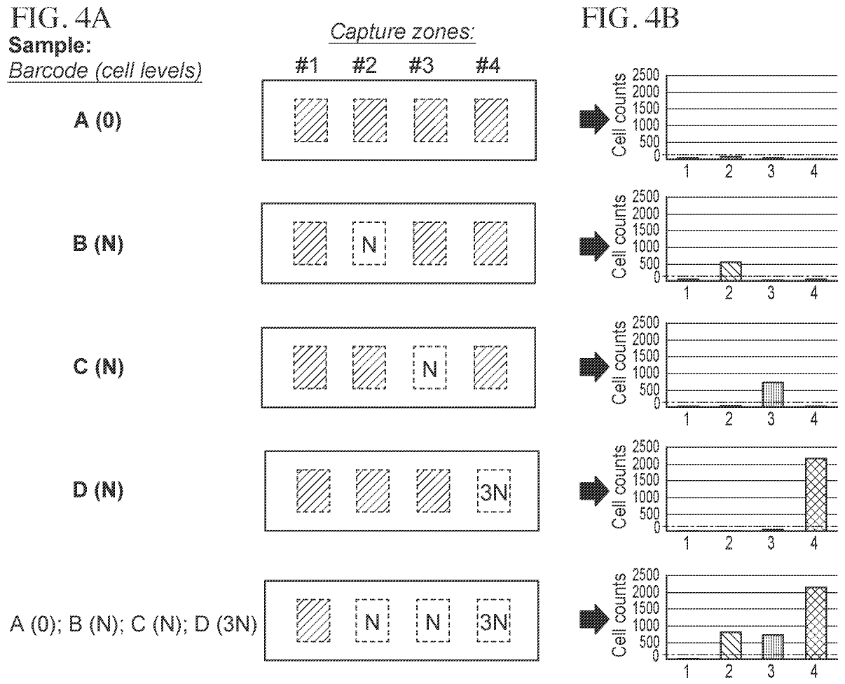

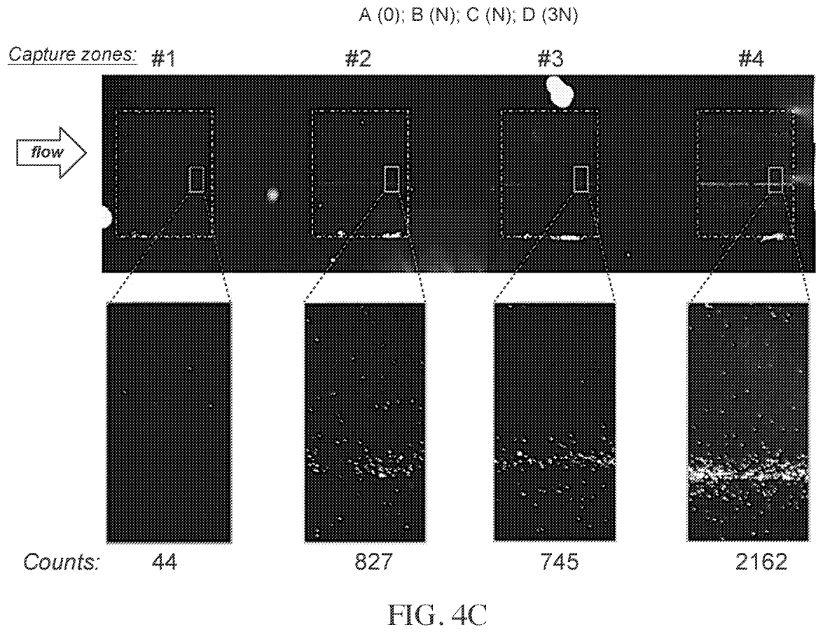

FIG. 4 depicts independent cell detection within pooled samples. Negative Sample A. FIG. 4A depicts a schematic of the experiment: each barcode was independently incubated with samples containing various loads of Salmonella cells(N, 3N or zero). Each set of Capture Zones are present in each lane and the direction of flow in the cartridge is from left to right. Barcode A (anti-ZipCode—conjugated antibody #1) was incubated with sample containing no Salmonella (0); Barcode B (anti-ZipCode—conjugated antibody #5) and Barcode C (anti-ZipCode—conjugated antibody #13) were incubated with 25,000 CFU per sample (N); and Barcode D (anti-ZipCode—conjugated antibody #25) was incubated with 75,000 CFU per sample (3N); was incubated with 75,000 CFU per sample (3N). After washing and recovery of the barcoded cells, each complex was run in a separate lane (top four lanes). In parallel, 4 barcoded samples prepared in the same way were combined (A (0); B(N); C(N); D (3N)) and run within the single channel (last lane). FIG. 4B depicts a graphic representation of the results for each lane. Colored bars represent cell counts within each capture zone (#1, zip oligo 1; #2, zip oligo 5; #3, zip oligo 13; and #4, zip oligo 25). Grey dotted line marks the count level corresponding system noise. Comparison of cell counts for pooled sample (A (0); B(N); C(N); D (3N) last graph) to the cell counts resulting from individual sample runs (top four graphs) demonstrates that the barcoded cell capture is orthogonal (the presence of cells in samples B, C and D did not affect the negative result in sample A). Moreover, the cell counts are indicative of the relative levels of Salmonella present in the sample: counts in B(N) and C (N) are similar, while counts in D (3N) are distinguishably higher than in either B or C. The cell counts obtained with the pooled sample are similar to the counts obtained with the individual samples. FIG. 4C depicts an image corresponding to the pooled sample A (0); B(N); C (N); D (3N). Positions of the capture zones are indicated by white dashed rectangles (#1, zip oligo 1; #2, zip oligo 5; #3, zip oligo 13; and #4, zip oligo 25). Auto-fluorescent signal from adhesive residue from the mask layer is visible at the edges of the capture zone. This area was excluded from analysis by the counting algorithm. The direction of the flow is shown with an arrow. A small segment of each capture zone is magnified to reveal fluorescent cells. The numbers under each segment are cell counts for the entire corresponding capture zone used to generate the graph above.

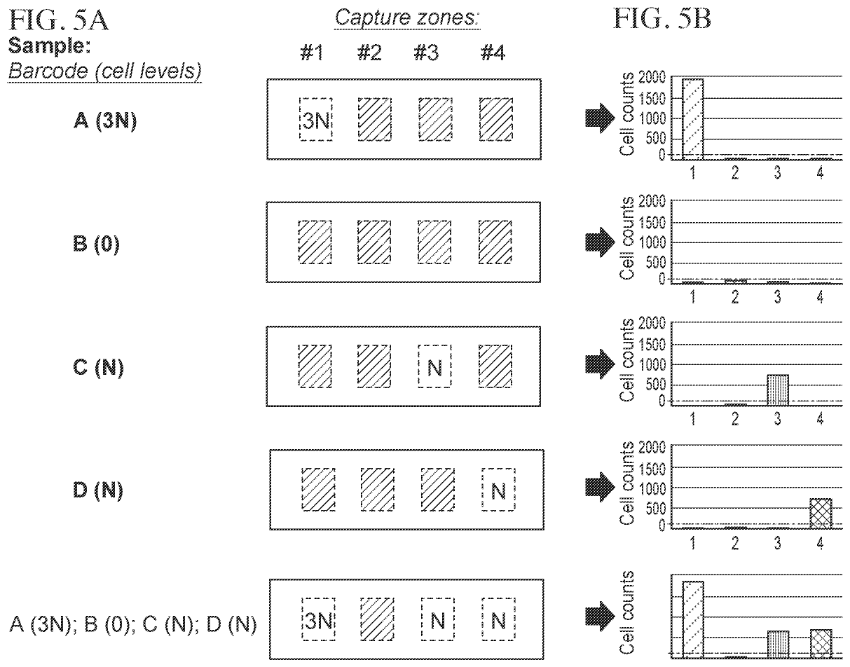

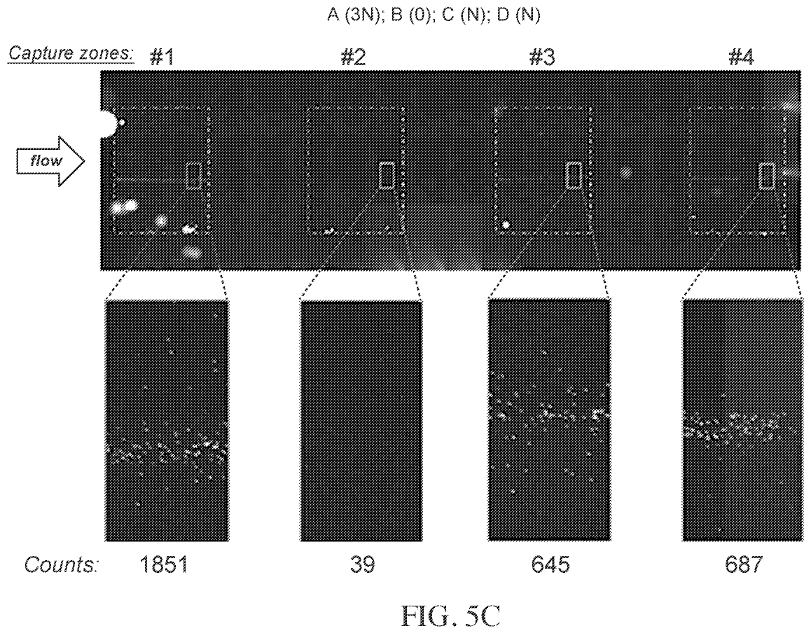

FIG. 5 depicts independent cell detection within pooled samples. Negative sample B. FIG. 5A depicts a schematic of the experiment: each barcode was independently incubated with samples containing various loads of Salmonella cells (N, 3N or zero). Each set of Capture Zones are present in each lane and the direction of flow in the cartridge is from left to right. Barcode A (anti-ZipCode—conjugated antibody #1) incubated with 75,000 CFU per sample (3N); Barcode B (anti-ZipCode—conjugated antibody #5) was incubated with sample containing no Salmonella (0); Barcode C (anti-ZipCode—conjugated antibody #13) and Barcode D (anti-ZipCode—conjugated antibody #25) were incubated with 25,000 CFU per sample (N).

After washing and recovery of the barcoded cells, each complex was run in a separate lane (top four lanes). In parallel, 4 barcoded samples prepared in the same way were combined (A (3N); B (0); C (N); D (N)) and run within the single channel (last lane). FIG. 5B depicts a graphic representation of the results for each lane. Colored bars represent cell counts within each capture zone (#1, zip oligo 1; #2, zip oligo 5; #3, zip oligo 13; and #4, zip oligo 25). Grey dotted line marks the count level corresponding system noise. Comparison of cell counts for pooled sample combined (A (3N); B (0); C (N); D (N) last graph) to the cell counts resulting from individual sample runs (top four graphs) demonstrates that the barcoded cell capture is orthogonal (the presence of cells in samples A, C and D did not affect the negative result in sample B). Moreover, the cell counts are indicative of the relative levels of Salmonella present in the sample: counts in C (N) and D (N) are similar, while counts in A (3N) are distinguishably higher than in either C or D. The cell counts obtained with the pooled sample are similar to the counts obtained with the individual samples. FIG. 5C depicts an image corresponding to the pooled sample A (3N); B (0); C (N); D (N). Positions of the capture zones are indicated by white dashed rectangles (#1, zip oligo 1; #2, zip oligo 5; #3, zip oligo 13; and #4, zip oligo 25). Auto-fluorescent signal from adhesive residue from the mask layer is visible at the edges of the capture zone. This area was excluded from analysis by the counting algorithm. The direction of the flow is shown with an arrow. A small segment of each capture zone is magnified to reveal fluorescent cells. The numbers under each segment are cell counts for the entire corresponding capture zone used to generate the graph above.

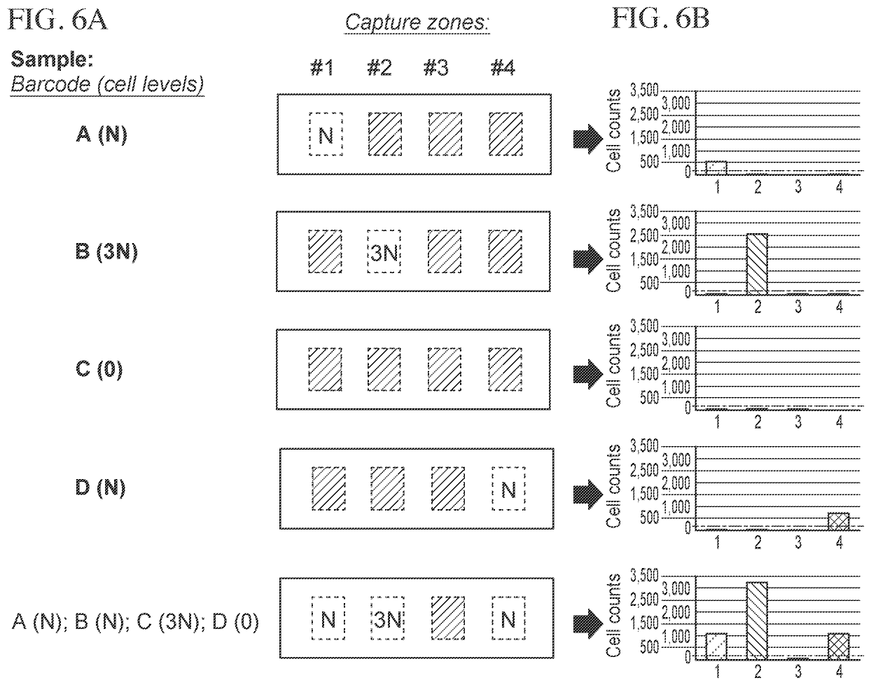

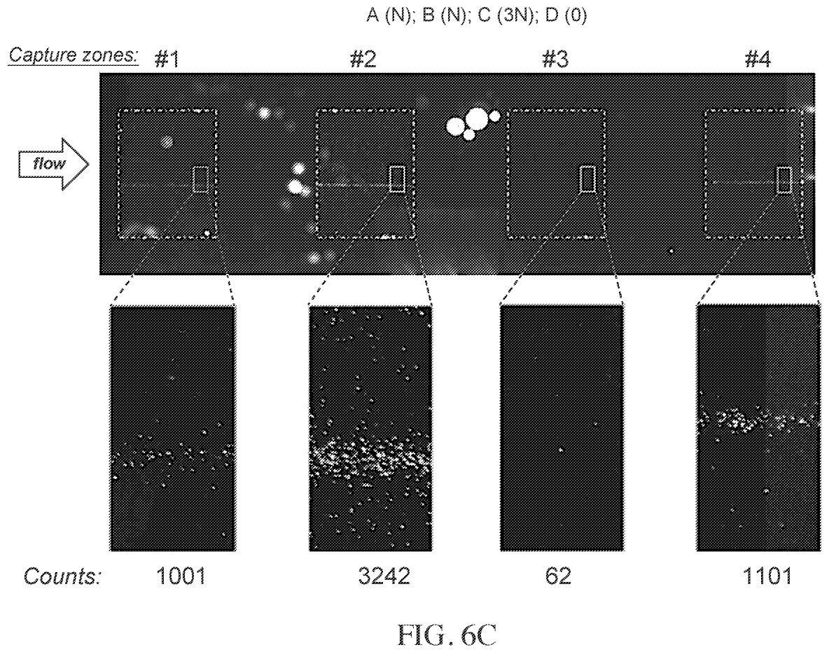

FIG. 6 depicts independent cell detection within pooled samples: example with negative sample C. FIG. 6A depicts a schematic of the experiment: each barcode was independently incubated with samples containing various loads of Salmonella cells (N, 3N or zero). Each set of Capture Zones are present in each lane and the direction of flow in the cartridge is from left to right. Barcode A (anti-ZipCode—conjugated antibody #1) incubated with 25,000 CFU per sample (N); Barcode B (anti-ZipCode—conjugated antibody #5) was incubated with 75,000 CFU per sample (3N); Barcode C (anti-ZipCode—conjugated antibody #13) was incubated with sample containing no Salmonella (0) and Barcode D (anti-ZipCode—conjugated antibody #25) was incubated with 25,000 CFU per sample (N). After washing and recovery of the barcoded cells, each complex was run in a separate lane (top four lanes). In parallel, 4 barcoded samples prepared in the same way were combined (A(N); B (3N); C(0); D (N)) and run within the single channel (last lane). FIG. 6B depicts a graphic representation of the results for each lane. Colored bars represent cell counts within each capture zone (#1, zip oligo 1; #2, zip oligo 5; #3, zip oligo 13; and #4, zip oligo 25). Grey dotted line marks the count level corresponding system noise. Comparison of cell counts for pooled sample combined (A(N); B (3N); C(0); D (N), last graph) to the cell counts resulting from individual sample runs (top four graphs) demonstrates that the barcoded cell capture is orthogonal. In other words, the presence of cells in samples A, B and D did not affect the negative result in sample C. Moreover, the cell counts are indicative of the relative levels of Salmonella present in the sample: counts in A (N) and D (N) are similar, while counts in B (3N) are distinguishably higher than in either A or D. FIG. 6C depicts an image corresponding to the pooled sample A(N); B (3N); C(0); D (N). Positions of the capture zones are indicated by white dashed rectangles (#1, zip oligo 1; #2, zip oligo 5; #3, zip oligo 13; and #4, zip oligo 25). Auto-fluorescent signal from adhesive residue from the mask layer is visible at the edges of the capture zone. This area was excluded from analysis by the counting algorithm. The direction of the flow is shown with an arrow. A small segment of each capture zone is magnified to reveal fluorescent cells. The numbers under each segment are cell counts for the entire corresponding capture zone used to generate the graph above.

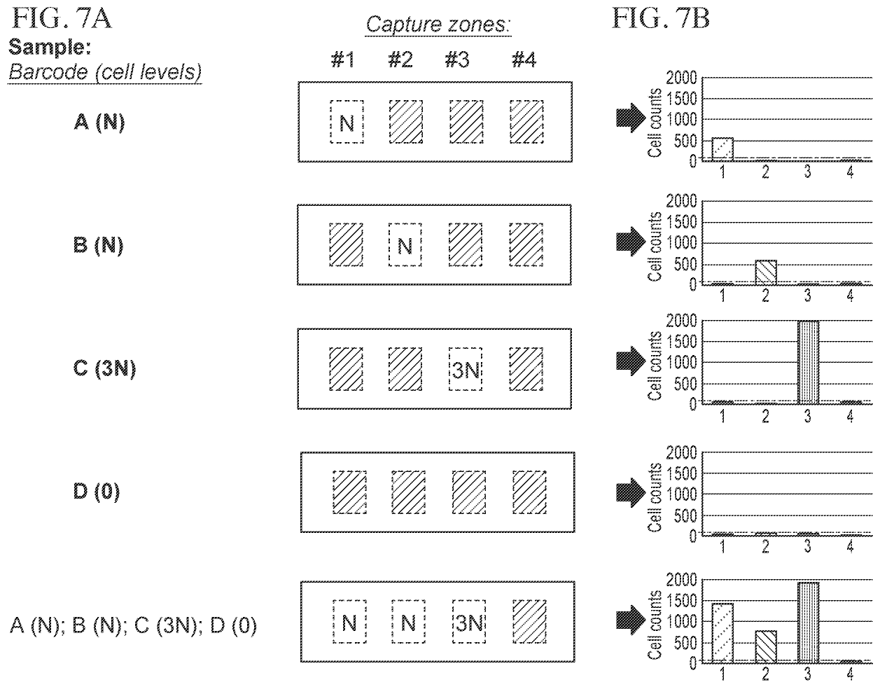

FIG. 7 depicts independent cell detection within pooled samples: example with negative sample D. FIG. 7A depicts a schematic of the experiment: each barcode was independently incubated with samples containing various loads of Salmonella cells (N, 3N or zero). Each set of Capture Zones are present in each lane and the direction of flow in the cartridge is from left to right. Barcode A (anti-ZipCode—conjugated antibody #1) and Barcode B (anti-ZipCode—conjugated antibody #5) were incubated with 25,000 CFU per sample (N); Barcode C (anti-ZipCode—conjugated antibody #13) was incubated with 75,000 CFU per sample (3N); and Barcode D (anti-ZipCode—conjugated antibody #25) was incubated with sample containing no Salmonella (0). After washing and recovery of the barcoded cells, each complex was run in a separate lane (top four lanes). In parallel, 4 barcoded samples prepared in the same way were combined (A (N); B (N); C (3N); D (0)) and run within the single channel (last lane). FIG. 7B depicts a graphic representation of the results for each lane. Colored bars represent cell counts within each capture zone (#1, zip oligo 1; #2, zip oligo 5; #3, zip oligo 13; and #4, zip oligo 25). Grey dotted line marks the count level corresponding system noise.

Comparison of cell counts for pooled sample combined (A (N); B (N); C (3N); D (0), last graph) to the cell counts resulting from individual sample runs (top four graphs) demonstrates that the barcoded cell capture is orthogonal (the presence of cells in samples A, B and did not affect the negative result in sample D).

Moreover, the cell counts are indicative of the relative levels of Salmonella present in the sample: counts in B (3N) are distinguishably higher than in either A or B. In this experiment, however, the counts in samples A (N) and B (N) were not as close as in other examples. This outlier result could be due variability of the hand coating and assembly of the cartridge prototype. FIG. 7C shows an image corresponding to the pooled sample A (N); B (N); C (3N); D (0). Positions of the capture zones are indicated by white dashed rectangles (#1, zip oligo 1; #2, zip oligo 5; #3, zip oligo 13; and #4, zip oligo 25). Auto-fluorescent signal from adhesive residue from the mask layer is visible at the edges of the capture zone. This area was excluded from analysis by the counting algorithm. The direction of the flow is shown with an arrow. A small segment of each capture zone is magnified to fluorescent cells. The numbers under each segment are cell counts for the entire corresponding capture zone used to generate the graph above.

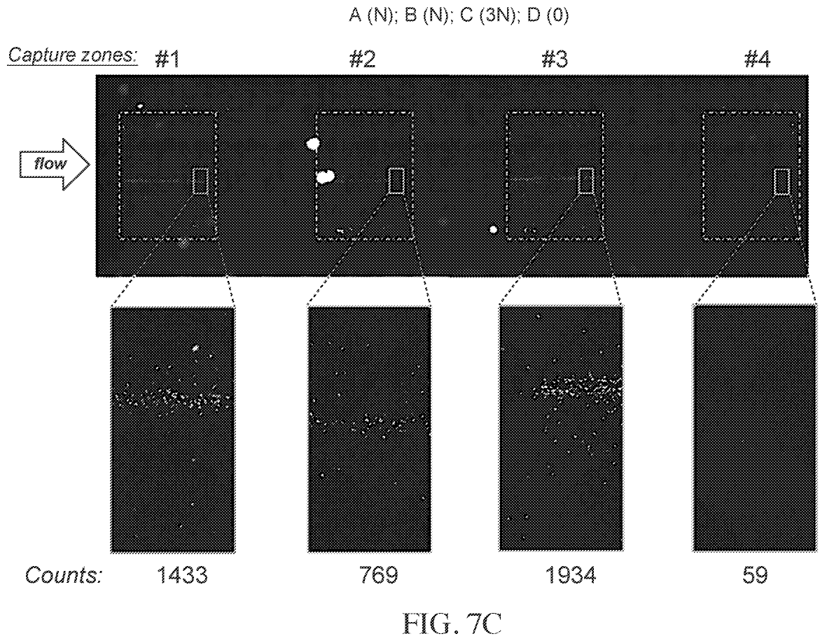

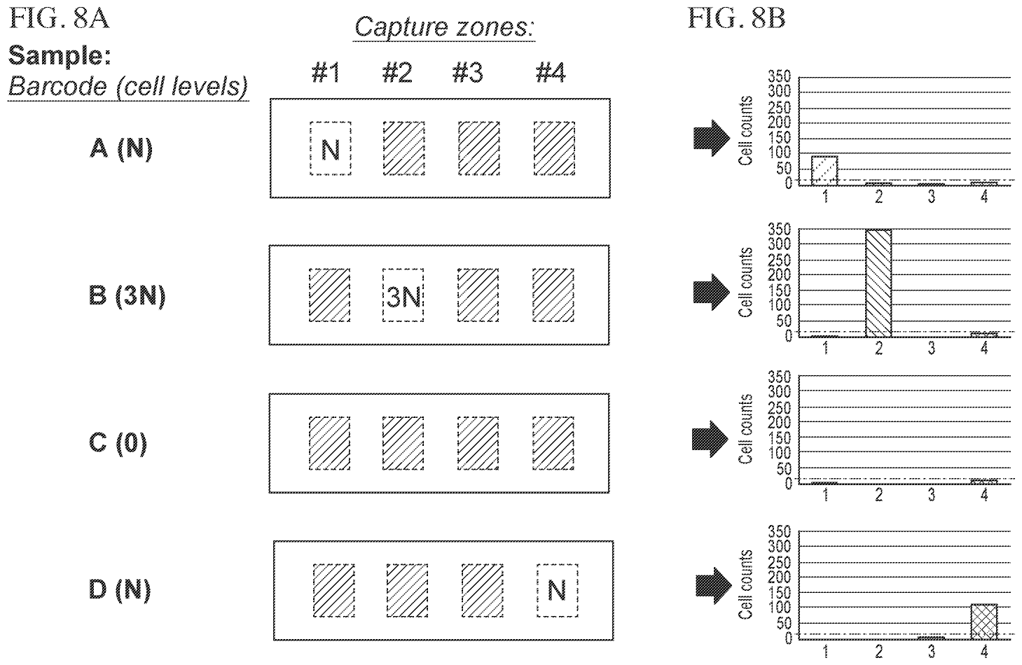

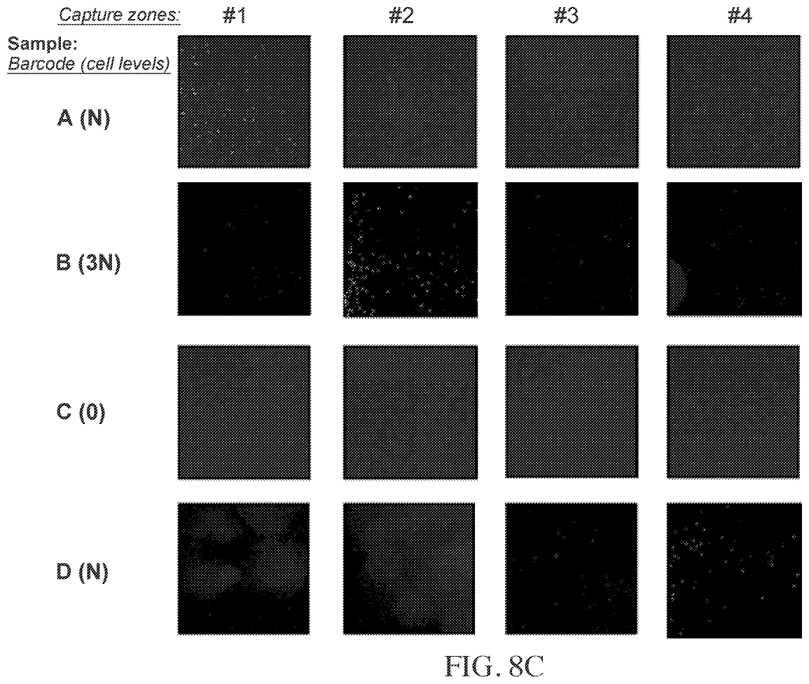

FIG. 8 depicts the analysis of poultry rinsate samples. FIG. 8A shows a schematic of the experiment: each barcode was independently incubated with poultry rinsate samples spiked with various loads of Salmonella cells (N, 3N or zero). Barcode A (anti-ZipCode—conjugated antibody #1) incubated with 25,000 CFU per sample (N); Barcode B (anti-ZipCode—conjugated antibody #5) was incubated with 75,000 CFU per sample (3N); Barcode C (anti-ZipCode—conjugated antibody #13) was incubated with sample containing no Salmonella (0) and Barcode D (anti-ZipCode—conjugated antibody #25) was incubated with 25,000 CFU per sample (N). After washing and recovery of the barcoded cells, each complex was run in a separate lane containing all four capture oligos. FIG. 8B shows a graphic representation of the results for each lane. Bar graphs represent cell counts within each capture zone (#1, zip oligo 1; #2, zip oligo 5; #3, zip oligo 13; and #4, zip oligo 25). FIG. 8C: A small segment of each capture zone is magnified to reveal fluorescent cells.

### Example #3A: Comparison Between Conventional FISH and 1-Step FISH

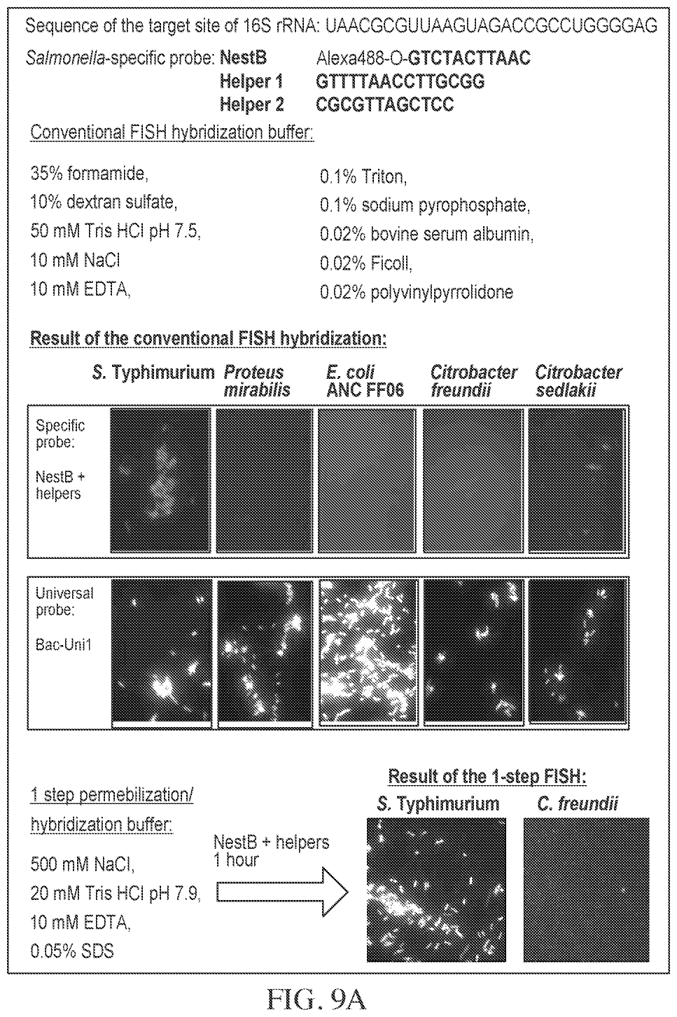

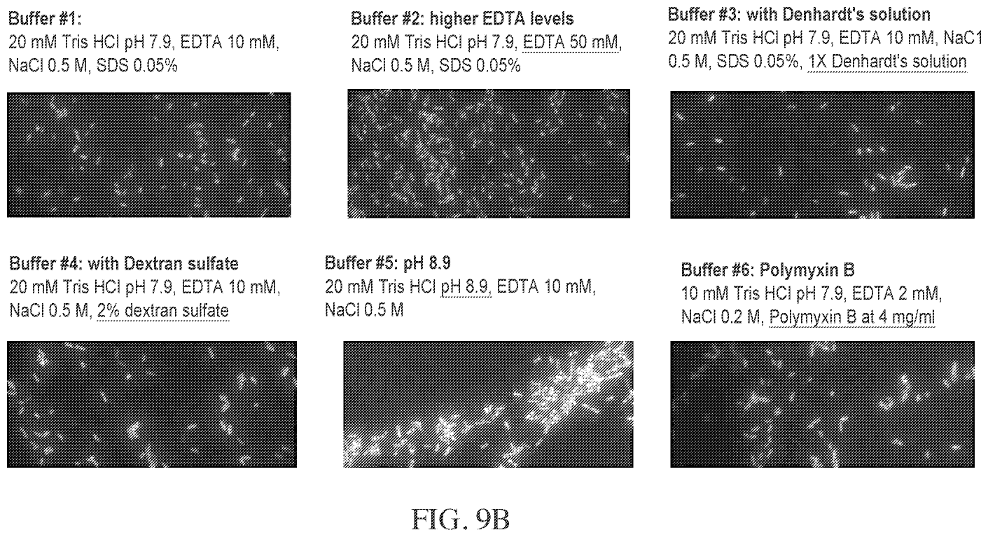

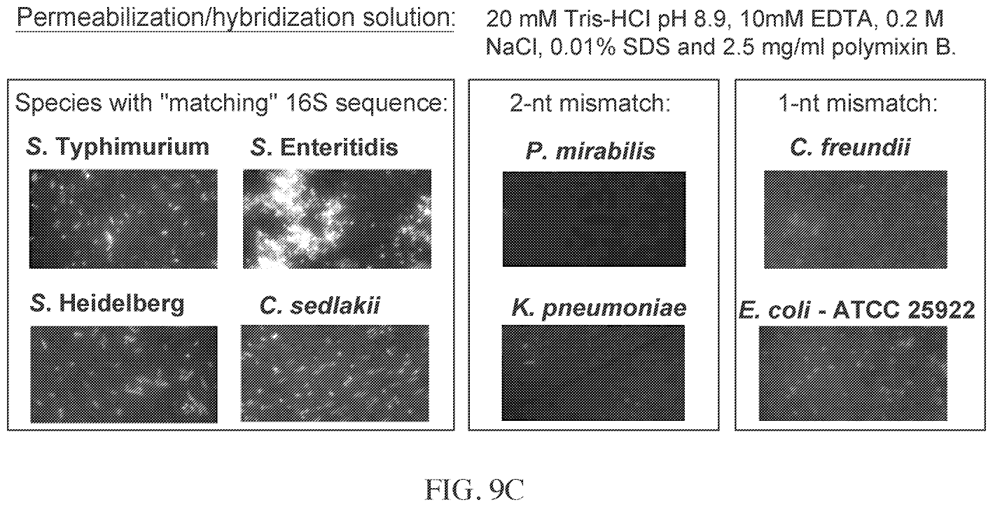

FIG. 9A shows a comparison between conventional FISH and the 1-step FISH described in this application. For this example, pure cultures of Salmonella enterica subsp. enterica sv. Typhimurium, (NCTC12023), or closely related bacterial species were grown for 4 hours in BPW at 42° C. from overnight culture.

For the figures in this application, the capture zone of the cartridge was coated with a broad specificity polyclonal anti-salmonella to capture all salmonella in a sample. The antibody was developed to capture 100 or more different salmonella serotypes. The antibody can also capture other types of bacteria that share surface antigens with salmonella, such as some serotypes of E. Coli or Citrobacter. Due to the specificity of the FISH probes, The FISH labeling describe herein only identifies salmonella thus increasing the accuracy of the method.

For the conventional FISH method, cells were fixed with 4% formaldehyde for 1 hour at RT. Next, cells were spun down at 6000 g for 5 min and supernatant was removed. Cells were resuspended in 1× PBS, then equal volume of ethanol was added for permeabilization. Cells were then pelleted by centrifugation, and then were re-suspended directly in hybridization buffer containing 35% formamide, 10% dextran sulfate, 50 mM Tris HCl pH 7.9, 10 mM EDTA, NaCl 10 mM, 0.1% Triton, 0.1% sodium pyrophosphate, 1×Denhardt's solution and PNA probes (0.4 μM NestB+1 μM helpers 1 & 2 or 0.4 μM Bac-Uni1). Mixtures were incubated at 42° C., 46° C. (shown in FIG. 9A, Results of the Conventional FISH Hybridization) and 50° C. using the gradient feature in PCR machine for 18h. Cell suspensions were pipetted directly onto glass slides and visualized under the setting appropriate for Alexa Fluor-488 dye on the fluorescent microscope. Under these conditions Salmonella grows faster than Citrobacter or E. coli. Proteus appear to grow just as fast as Salmonella. Slower growth of Citrobacter and E. coli in this experiment likely affects the number of ribosomes per cell and overall brightness in FISH experiments even under saturating conditions.

For the 1-step FISH method, cells were concentrated by centrifugation and mixed directly with hybridization buffer (0.5 M NaCl, 0.05% SDS, 20 mM Tris pH 7.9, 10 mM EDTA) and incubated at 42, 46 (shown in FIG. 9A, Results of the 1-Step FISH) or 50° C. for 1 hour. Cells suspensions were pipetted directly onto glass slides and visualized under the setting appropriate for Alexa Fluor-488 dye on fluorescent microscope.

Probes: 400 nM of Alexa Fluor-488 PNA (Salmonella-specific or Universal), 1 μM PNA helper probes where indicated.

### Example #3B: Various Buffer Formulations for 1-Step FISH

Experimental setup: Salmonella Typhimurium was grown at 42° C. to early exponential phase in BPW, no agitation. Cells were concentrated by centrifugation and resuspended in buffer containing 400 nM of Bac-Uni1 universal bacterial probe. Buffer composition is indicated for each condition.

After 1 h at 46° C. cell suspensions were spotted directly on the glass slide and visualized at gain 10 and 300 ms exposure on fluorescent microscope set for Alexa Fluor 488 dye.

### Example #3C: Polymyxin B can be Used as a Permeabilization Reagent

Polymyxin B is a peptide anti-bacterial agent selective against Gram-negative bacteria. Its mechanism of action involves penetrating the lipid layer of the outer bacterial membrane, disrupting its integrity. In this Example, it is shown that Polymyxin B can be used as a permeabilization agent in a 1-step assay although the difference between the specific and non-specific signal was somewhat lower compared to high-salt and 0.05% SDS-based formulation.

We compared cell labeling with Salmonella-specific probe NestB of several Salmonella organisms and one non-Salmonella organism that are a “match” to the probe (Salmonella Typhimurium, Salmonella Heidelberg, Salmonella Enteritidis, and Citrobacter sedlakii) as well as bacteria containing “mismatched” sequences within their ribosomes (Proteus mirabilis, Klebsiella pneumoniae, Citrobacter freundii, E. coli).

All strains were grown at 42° C. to early exponential phase in BPW, no agitation. Cells were concentrated by centrifugation and resuspended in buffer containing 400 nM of NestB, 1 μM helpers, 20 mM Tris-HCl pH 8.9, 10 mM EDTA, 0.2 M NaCl, 0.01% SDS and 2.5 mg/ml polymixin B. After 2 h at 42° C. cell suspensions were spotted directly on the glass slide and visualized on fluorescent microscope set for Alexa Fluor 488 dye.

### Example #4A: On-Cartridge Labeling with PNA-FISH of Pre-Fixed Cells Demonstrated Increase in Specificity Compared to SYBR Green Staining—PNA-FISH Labeling is Compatible with Ferrofluid Based Microfluidic Cartridge Platform

Fixed cells were prepared as described in Example 1A.

About 0.5 million cells for Salmonella Typhimurium cells, or 5 million cells for Citrobacter freundii cells, or 5 million cells Proteus mirabilis were mixed with PEG ferrofluid and loaded into the sample reservoir on a cartridge.

For labeling, 400 nM of NestB with 1 uM helpers, or Bac-Uni1 probes were mixed in buffer containing 20 mM Tris-HCl pH 7.9, 10 mM EDTA, 0.5 M NaCl and 0.05% SDS and loaded into the cartridge reagent reservoir. Labeling conditions were set at 45° C. for 1 h.

SYBR Green was used as a labeling agent for comparison. Images were taken at 600 ms exposure for probe-labeled samples, and 100 ms for SYBR-labeled samples

### On-Cartridge Labeling of Live Cells Demonstrated the Specificity of 1-Step PNA-FISH

Strain handling:
1. Strains from the frozen stocks were streaked out on TSA plates and incubated overnight at 37° C.
2. Colonies from fresh plates were inoculated into 10 ml of BPW and grown for 7 hours at 42° C. with no agitation (reach stationary phase after 3 hours).
3. Salmonella strains were diluted 100× prior to the ferrofluid-based microfluidic run,
4. Non-Salmonella strains were used as is.

Preparation of samples and reagents (within ˜20 min of cartridge run):

1. Bacterial cultures prepared as described above were mixed 1:1 with PEG ferrofluid. Pre-stained heat killed Salmonella cells were used as antibody performance control.

2. “Specific” labeling solution contained 1 μM of NestB probe, 2 μM of helper 1, and 2 μM of helper 2 in labeling buffer (20 mM Tris-HCl pH 7.9, 10 mM EDTA, 0.5 M NaCl and 0.05% SDS).

3. “Non-specific” labeling solution contained 1 μM of Bac-Uni1 probe in the same labeling buffer (25 mM Tris-HCl pH 7.9, 15 mM EDTA, 0.5 M NaCl and 0.02% SDS). Labeling with Bac-Uni1 allowed the experiment to assess the specificity of NestB.

4. SYBR-Green control: 10,000× concentrated solution of SYBR-Green was diluted 100 times in growth media (BPW) immediately before use.

### Ferrofluid-Based Microfluidic Cartridge Run:

1. 500 μL of each sample (bacterial cultures mixed with PEG ferrofluid) were loaded into the assigned sample reservoir on each of the three cartridges. Each sample was analyzed with three different labeling reagents: “specific”, “non-specific” and SYBR-green

2. 100 μL of each of the labeling reagents was loaded into reagent reservoirs on the corresponding cartridge.

3. Labeling conditions for molecular probes (specific and non-specific): initial reagent flow for 6 min 15 sec at 10 μL/min at 48° C., followed by 5 min no-flow incubation at 48° C. and 25 min no-flow incubation at 42° C. Images were collected at 600 msec exposure.

4. Labeling conditions for SYBR Green: one-step incubation at 39° C. for 5 minutes. Images were collected at 100 msec exposure.

### Example #5: Validation Studies of the FISH Assays

Biocompatible ferrofluids stabilized with surfactant (EMG series from Ferrotec) are used in the FISH detection assays of the present disclosure.

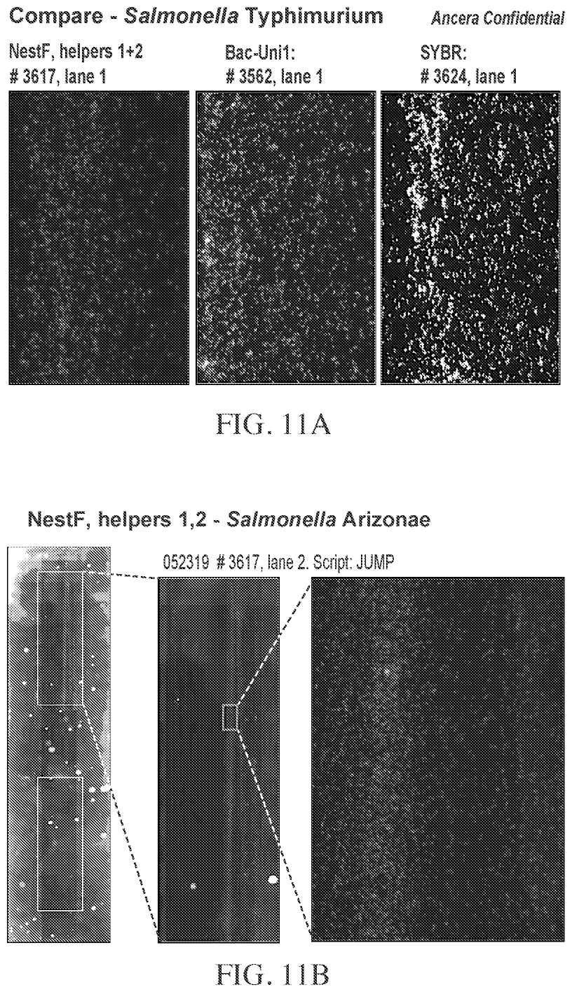

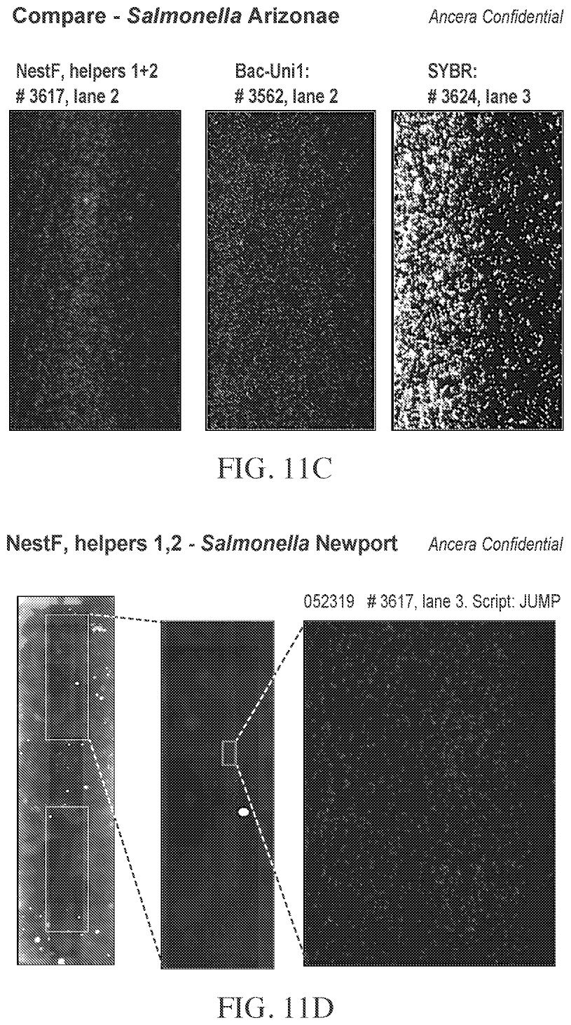

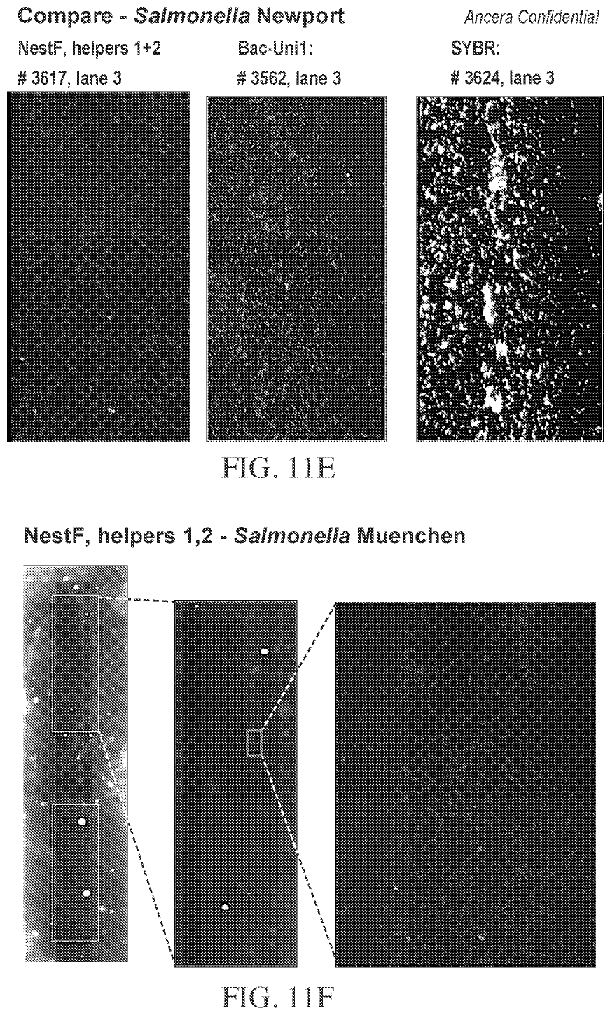

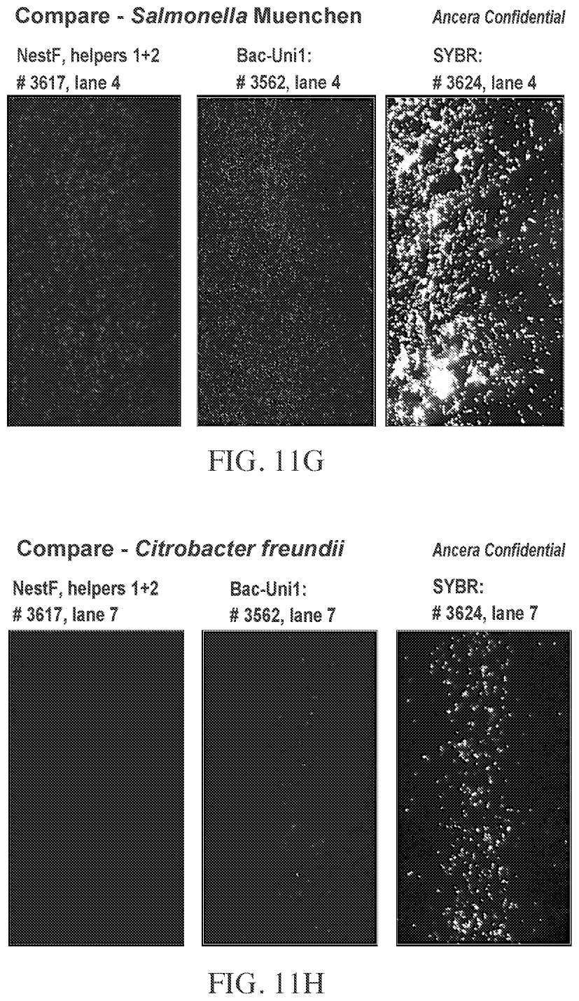

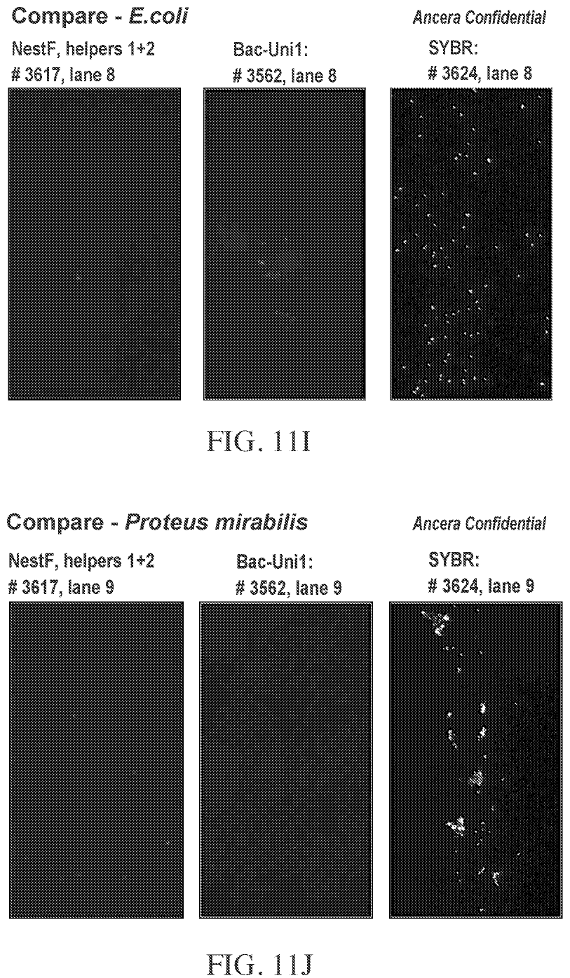

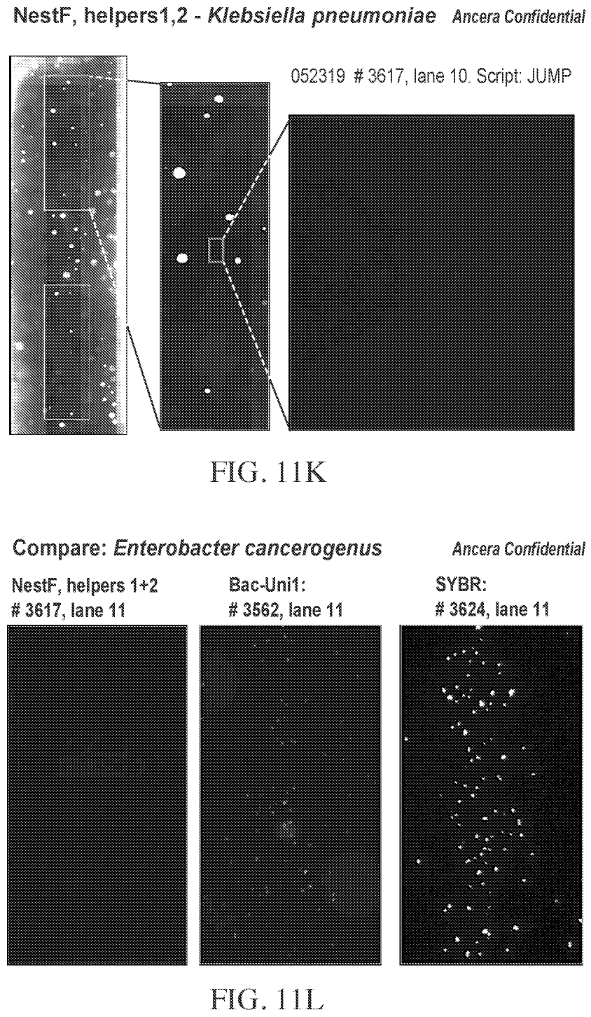

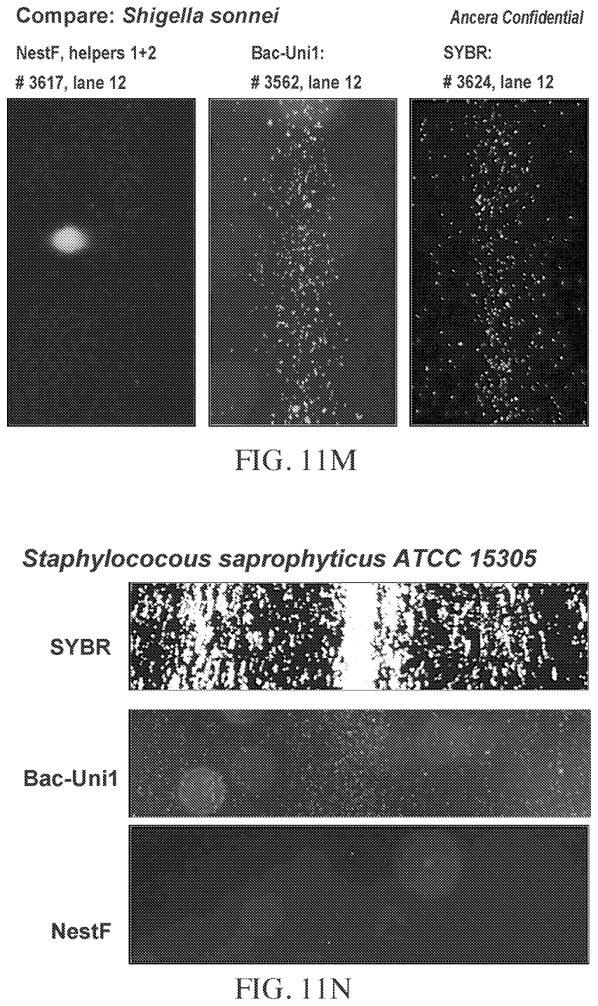

For the studies described below, a 1.7 mL/cartridge of the labeling reagent was prepared by mixing 1040 μL of Base Buffer (350 mM EDTA, 75 mM Tris-C1, pH 8), 100.75 μL of Molecular Biology grade water, 13 μL each of 2 Helper Probes (100 μM in 50% DMF, 100 mM Tris-C1, pH 8), 3.25 μL of Alexa Fluor-488 labeled PNA Probe F (GTCTACTTAAC-Lys-Alexa 488, 200 μM in 50% DMF, 100 mM Tris-C1, pH 8), and, 130 μL of 0.2% SDS solution. The solution was mixed by inverting the tube 10 times. 95 μL was loaded in the detection reagent reservoir of each lane. Samples (125 μL of ferrofluid, 125 μL of phosphate buffered saline, and 250 μL of diluted or enriched cells) were added to each sample reservoir. The cartridge was then processed. Cartridge flow was at 25 uL per minute for 40 minutes to allow cells to be captured in the capture zone of each lane. For FISH hybridization the temperature of the cartridge was raised to 48° C. for 5 minutes, and then to 42° C. for 20 minutes. Images were processed with proprietary software which enables enumeration of cells in each zone (FIG. 11)).

### Inclusivity of the Surfactant-Based Ferrofluid Assay

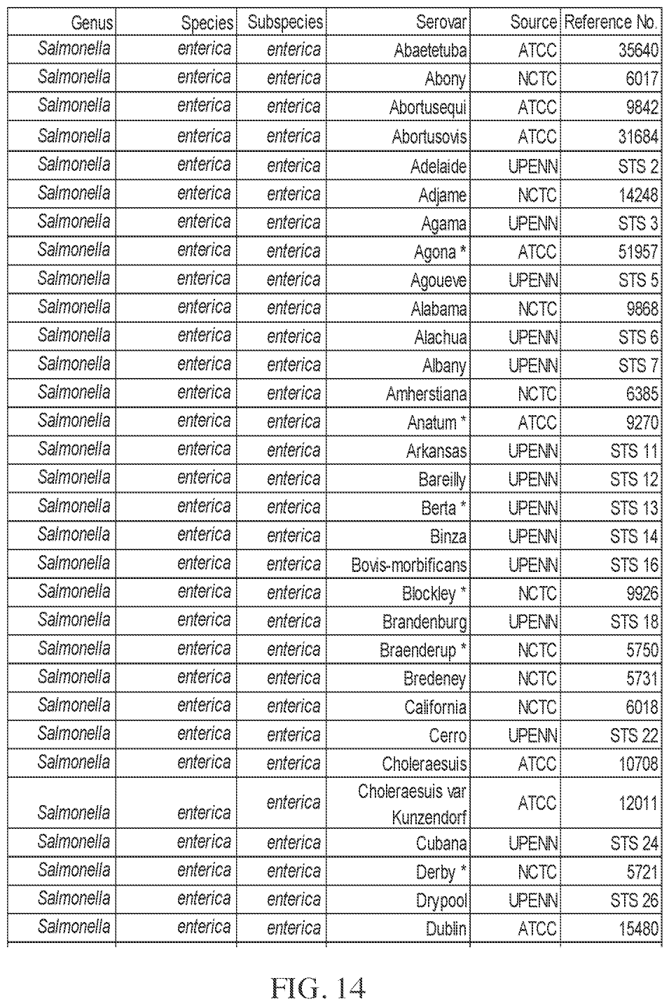

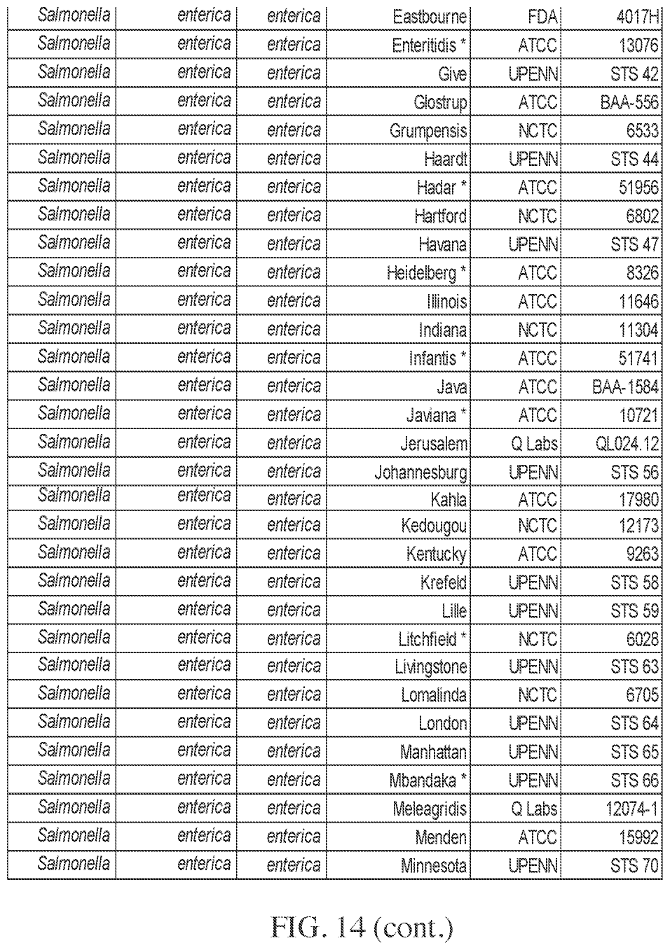

In a validation study, every strain of 100 serovars of Salmonella enterica subsp. enterica was detected by our FISH assay. These results are shown in FIG. 13. The list of inclusivity strains is described in FIG. 14. The serovars on the CDC list of the most epidemiologically important are noted with an asterisk.

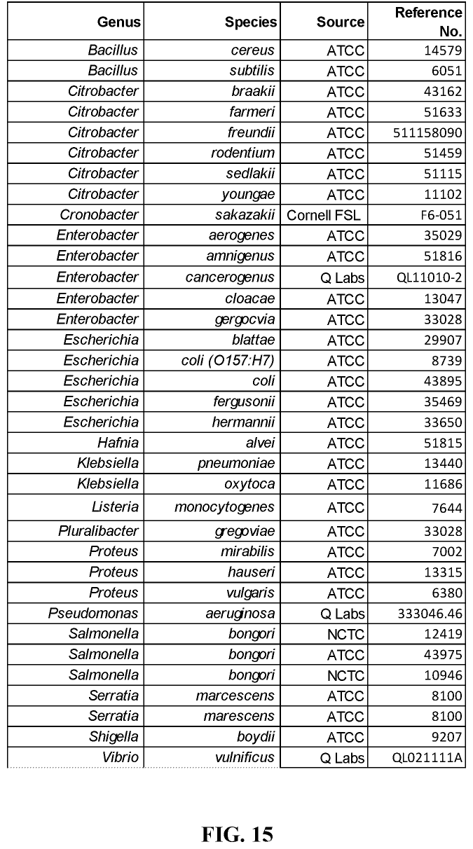

Next, the exclusivity of the assay was tested. The exclusivity list was comprised of closely related bacterial species or those commonly found in the environmental or poultry samples (FIG. 15). None of the 31 exclusivity strains yielded a positive result (FIG. 13 and FIG. 15), despite being added at over a 100-fold higher concentration compared to the Salmonella strains. This additional exclusivity is due to the capture antibody used to coat the capture zone in the Piper cartridge.

### Examples of Dose-Dependent Capture and Detection

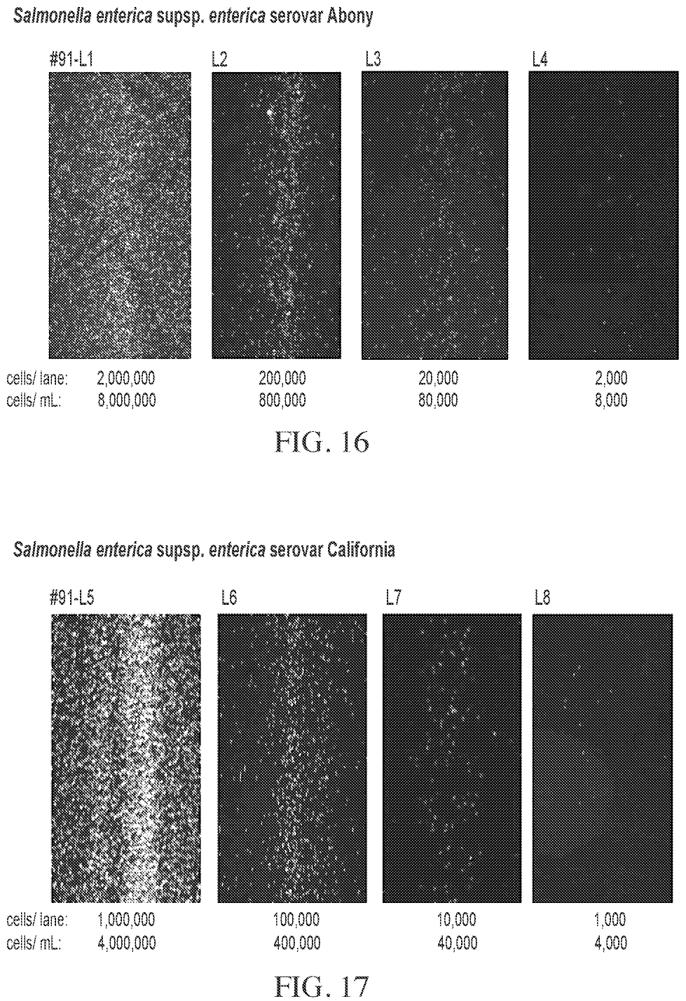

The detection of Salmonella enterica subsp. enterica in a dose-dependent manner is additionally shown. Ten-fold dilutions of several Salmonella serovars were prepared and processed using the FISH method. Selected images or cell counts obtained by image processing are shown in FIGS. 16 and 17.

### Cell Capture and Detection in Food Matrices or Environmental Samples is Efficient and Dose-Dependent.

The ability of the methods and kits of the present disclosure to detect salmonella in common food matrices and environmental samples is shown. First, the experiments demonstrate that there is no interference when testing poultry rinse or ground turkey samples. Serial dilutions of 3 Salmonella serotypes, S. Typhimurium, S. Enteritidis and S. Kentucky were tested, and the experiments showed no significant difference between the cell counts obtained with cells diluted in media alone or the sample matrices (FIG. 18).

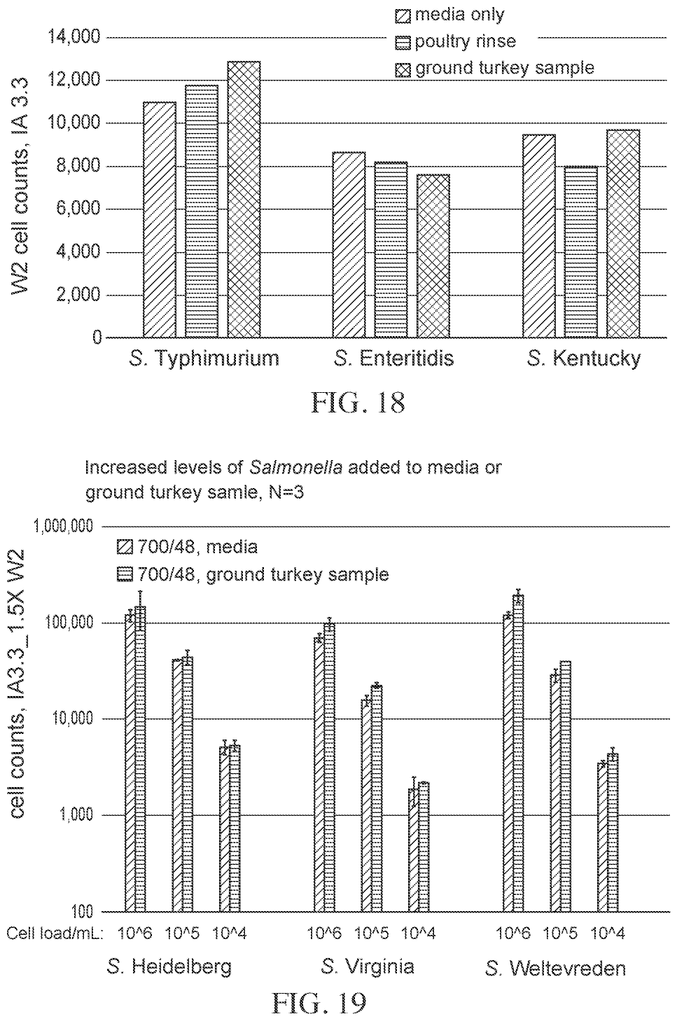

Additionally, the dose dependent cell count is not affected by sample matrix. Serial dilutions of S. Heidelberg, S. Virginia and S. Weltevreden were diluted in pure media and in ground turkey suspension (1-25 in media). Results show no interference in the ability to get a cell number specific signal in the pure media or ground turkey sample (FIG. 19).

Next, the assay of the present disclosure can detect naturally occurring Salmonella in food matrices or environmental samples. We enriched and tested a number of samples without adding Salmonella.

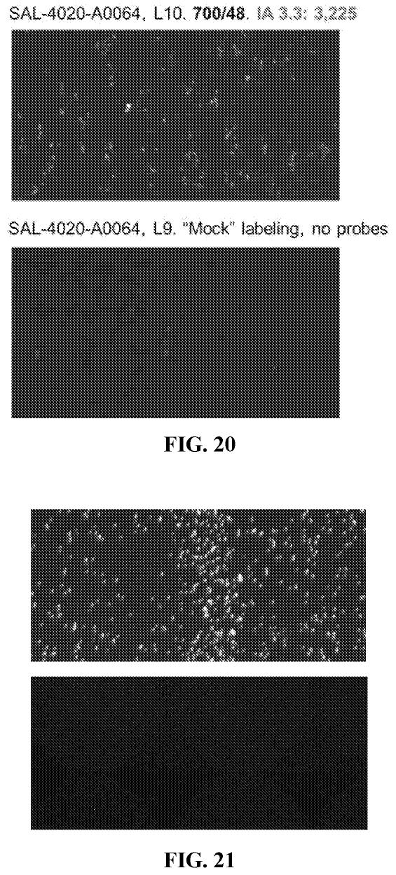

All unspiked cultures were tested in parallel and by the reference method BAM MLG 4.10 to confirm presence or absence of naturally occurring Salmonella. Representative images are shown in FIGS. 20 and 21.

### Example #6: Listeria FISH Assay

The Listeria Assay was performed using a MagDrive enabled disposable cartridge. Listeria and exclusivity strains were obtained from ATCC and other sources. Listeria strains were grown in sterile Brain Heart Infusion Media (Sigma Aldrich part #53286-500 g). PEG ferrofluid was prepared with TrueBlack (dissolved in DMSO) at a final concentration on 0.55% immediately prior to use. Samples were prepared by mixing cultures 1:1 with PEG ferrofluid. Cartriges were coated with a Listeria polyclonal antibody in the capture zone of each lane. Prepared samples were loaded into sample reservoirs of individual lanes of the cartridge/system, approximately 500 uL/lane.

Samples were pumped into the cartridge, pushed to the capture surface by a controlled magnetic field induced by an on-board PCB. The cartridge was maintained at 39° C. for 41.5 minutes during the “capture” step.

After capture, approximately 100 uL of labeling reagent was automatically flowed through each lane of the cartridge for 1 hr at 48° C. The labeling reagent contained a solution of PNA and DNA probes and a detergent to permeabilize cells. Specifically, the dye reagent contained 150 mM EDTA, 50 mM Tris-C1, 0.01% SDS, 250 nM PNA Lis240D, (listeria specific) 1000 nM PNA LisUn-3 H2 (helper probe) and 1250 nM DNA Lis240D-DQ (quencher probe) (See Table 3 for probe sequences). Lis240D-DQ is designed to bind to the Lis240D probe at low temperature to quench the fluorescence of probe which is not bound to a full-length target sequence.

TABLE 3

| ID | Type | Sequence (N′-C′ or 5′-3′)* |
|---|---|---|
| Lis240D | PNA | Alexa488-TAGCCGAAACCAT |
| LisUn-3 H2 | PNA | EE-ATTCGGTATTAGC-EE |
| LIS240D-DQ | DNA | TTCGGCTA-Dabcyl |

PNA base “E” is a solubility enhancer group.

After completion of the hybridization step, each lane was illuminated with blue LED excitation light and appropriate optics to stimulate green fluorescence of the Alexa Flour 488 labeled cells. Samples were imaged for 600 ms and the images were processed using a cell counting algorithm.

### Example #6A: Listeria Exclusivity

An experiment was performed to ensure exclusivity of the probe set. Twelve cultures were prepared and tested as described above. Included in the test were 3 strains of Listeria seeligeri derived from various sources as well as 1 strain of Listeria grayi and 8 strains of Non-Listeria bacteria. A count was automatically generated for each lane of the cartridge. At the time of the experiment a background threshold level of 500 counts was deemed necessary to call a sample positive. The results are presented in Table 4.

TABLE 4

| Lane | Species | Culture ID | Counts | Result |
|---|---|---|---|---|
| 1 | Shigella flexneri | ATCC 9699 | 17 | − |
| 2 | Streptococcus pyogenes | ATCC 19615 | 101 | − |
| 3 | Escherichia coli | ATCC 25922 | 20 | − |
| 4 | Listeria grayi | ATCC 25400 | 9 | − |
| 5 | Staphylococcus epidermidis | ATCC 12228 | 11 | − |
| 6 | Shigella sonnei | QL29930 | 14 | − |
| 7 | Salmonella enterica | ATCC 8326 | 7 | − |
| 8 | Listeria seeligeri | ATCC 51335 | 997* | +++ |
| 9 | Listeria seeligeri | QL 031011-1 | 98493 | +++ |
| 10 | Hafnia alvei | Va12-1a-1.1 | 13 | − |
| 11 | Listeria seeligeri | ATCC 35967 | 998* | +++ |
| 12 | Enterococcus faecalis | ATCC 33186 | 25 | − |

*Samples were not appropriately counted by the algorithm due to an overabundance of cells in the detection window.

As can been seen in Table 4, all three samples containing L. seeligeri strains tested as highly positive by the test. The limitations of the cell counting algorithm at the time of analysis incorrectly called two of the strains as weakly positive where, in fact, they were strongly positive but miscounted. All images were inspected manually to confirm results. The one strain of L. grayi tested negative as was predicted by sequence alignment. The remaining 8 exclusivity strains representing 5 Gram-negative and 3 Gram-positive species all tested negative.

### Example #6B: Listeria Versatility

An experiment was performed to ensure inclusivity of the Lis240D probe for several Listeria species. In this case the labeled probe was used at 400 nM, the quencher probe, Lis240D-DQ was used at 1600 nM, and the helper probe, LisUn-3 H2, was not added. Samples were prepared in Half-Fraser media (Millipore-Sigma #69198) supplemented with nalidixic acid at 10 mg/L. Culture was performed as described in Example 1. Samples were tested undiluted, except Listeria strains which were diluted 100-fold in media prior to dilution with ferrofluid, L. seeligeri was diluted 20-fold. Samples were otherwise tested as described above. Results are shown in Table 5.

TABLE 5

| Lane | Species | Culture ID | Counts | Result |
|---|---|---|---|---|
| 5 | Listeria monocytogenes | SLR SLR2249 | 29274 | +++ |
| 6 | Listeria seeligeri | QL 031011-1 | 33 | − |
| 7 | Listeria innocua | ATCC 43547 | 48705 | +++ |
| 8 | Listeria ivanovii | BAA-139 | 9254 | +++ |
| 9 | Streptococcus mutans | ATCC 35668 | 2 | − |
| 10 | Staphylococcus aureus | (not recorded) | 1 | − |
| 11 | Enterococcus faecalis | ATCC 33186 | 86 | − |
| 12 | Hafnia alvei | Va12-1a-1.1 | 9 | − |

Samples containing L. monocytogenes, L. innocua or L. ivanovii produced bright positive signals. Although some cells could be seen in the sample image, the culture of L. seeligeri did not produce signal sufficient to call the sample positive. The other strains all failed to produce a positive result. E. faecalis was observed to bind to the sample window and was weakly fluorescent but did not produce signal at the level which qualified as a positive result according to the established cut-off set within the imaging algorithm. On inspection of the other images, none of the other Non-Listeria strains showed any significant binding to the sample window, or detection by the prepared probe set.

### Example #7: Signal Recovery and Fecal Analysis

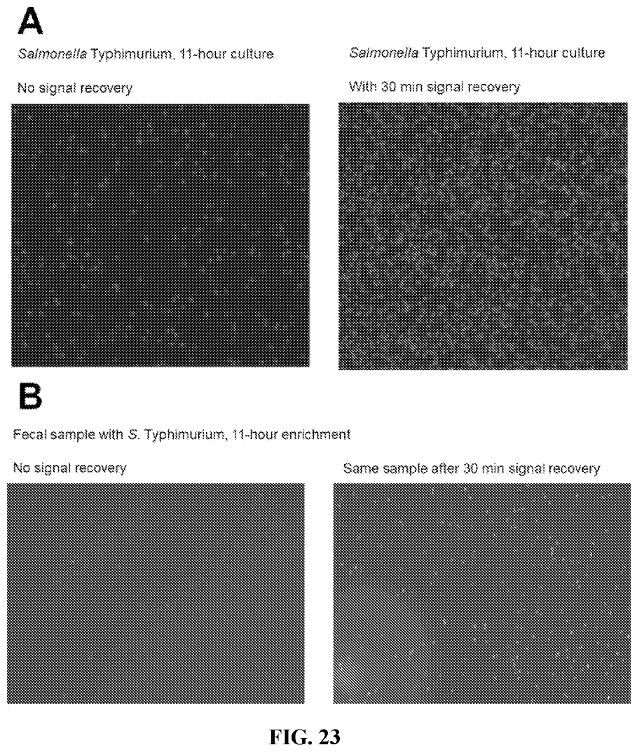

The effect of signal recovery treatment on (A) Salmonella Typhimurium grown for 11 hours as pure culture (FIG. 23A) or (B) spiked into fecal sample (1 g chicken feces per 200 mL of BPW media) is shown (FIG. 23B). An aliquot of the culture was run as is (left panels) or mixed 1:1 (v:v) with signal recovery media (glucose 200 mM, pyruvate 20 mM) and incubated at 42° C. for 30 min prior to run (right panels).

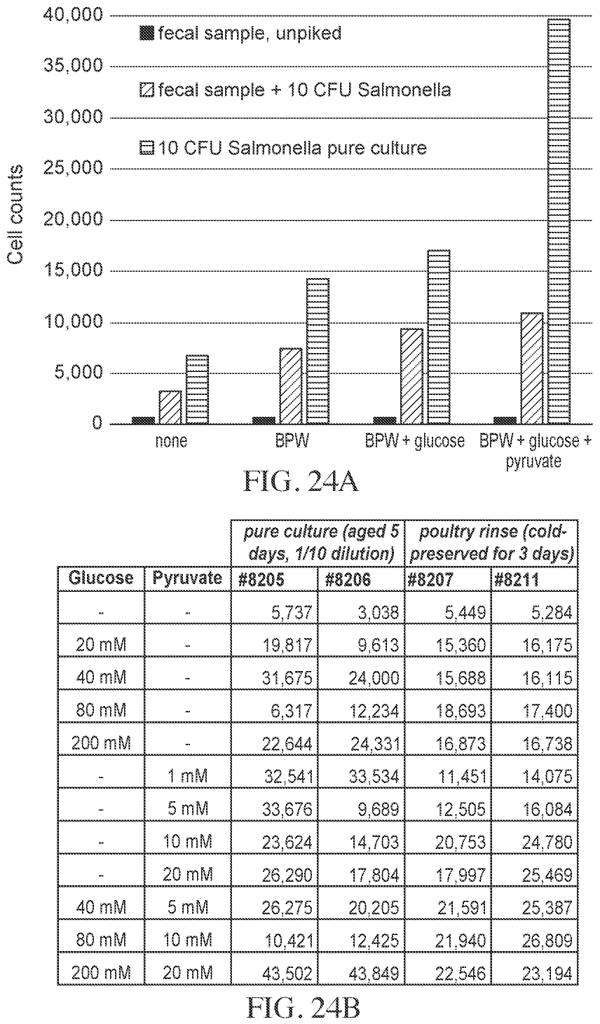

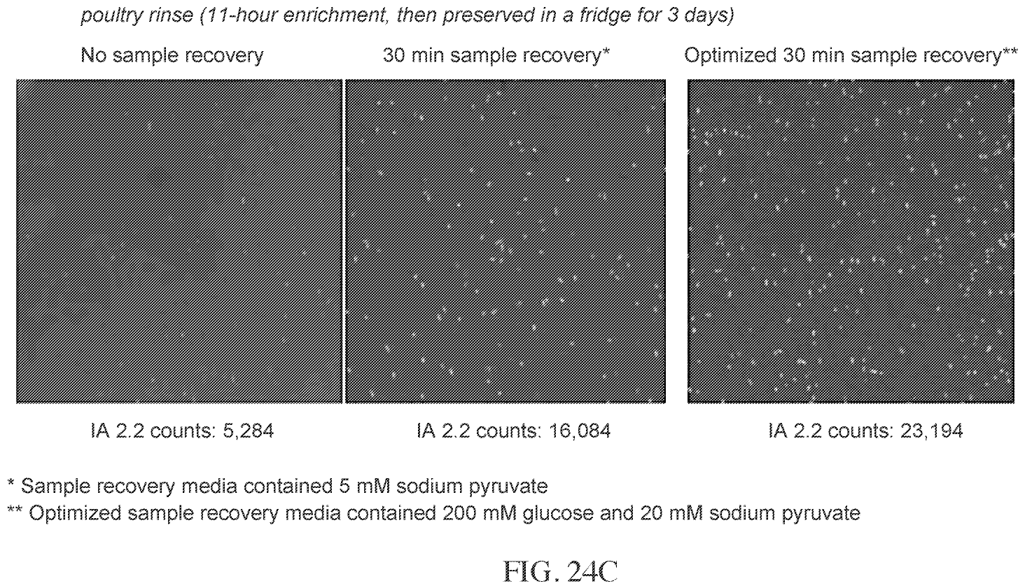

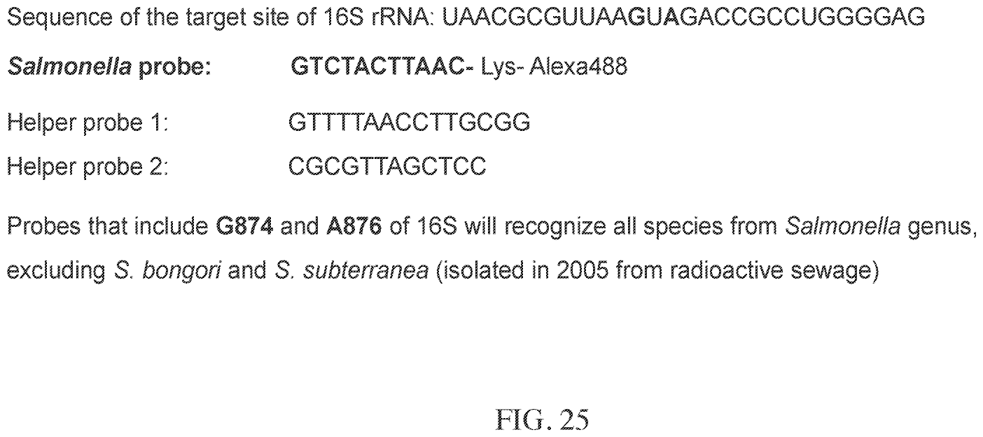

In both cases, signal recovery significantly increased the number of high-intensity cells detected. A) Signal recovery treatment on Salmonella Typhimurium grown for 11 hours as pure culture, grey bars, or spiked into fecal sample (1 g chicken feces per 200 mL of BPW media), striped bars is shown in FIG. 24A. The first set of bars shows cell counts as detected by the image analysis in untreated samples after the run. Next, an aliquot of the culture was mixed 1:1 (v:v) with media only (BPW) or media supplemented with 25 mM glucose, or BPW supplemented with 25 mM glucose an 0.5 mM sodium pyruvate; and incubated at 42° C. for 30 min prior to the run.

In all cases, signal recovery significantly increased the number of high-intensity cells detected by the algorithm, with the BPW supplemented with both glucose and pyruvate yielding the best results. Importantly, the count in fecal sample that lacked Salmonella (black bars) did not go increase in response to treatment, indicating that the signal recovery treatment does not compromise the assay specificity.

B) Optimization of Glucose and/or Pyruvate Levels in Signal Recovery Media (FIG. 24B)

To optimize the levels of the glucose and sodium pyruvate in signal recovery, we compared the counts of high-intensity cells as detected by the image recognition algorithm in aged samples subjected to different treatments. The first two columns correspond to experimental replicates for pure Salmonella Typhimurium culture that was stored for 5 days in the fridge prior to the experiment to induce metabolically depressed state. The culture was diluted 5 times in fresh BPW prior to treatments to reduce the number of cells. Two columns on the right correspond to experimental replicates for poultry rinsate spiked with Salmonella Typhimurium, at 42° C. for 11 hours, and subjected to cold storage for 3 days to induce metabolically depressed state in bacterial cells.

An aliquot of the culture or sample was run as is (−), or mixed 1:1 (v:v) with BPW media supplemented with glucose (20-200 mM), or sodium pyruvate (1-20 mM), or both; and incubated at 42° C. for 30 min prior to run.

In all cases, signal recovery significantly increased the number of high-intensity cells detected, Overall, the increased levels of either glucose or pyruvate corresponded to higher number of cells detected by the algorithm, with both supplements used together yielding the best result.

C) Example images from the optimization experiment (FIG. 24C). A small portion of the images was selected to show fluorescently labelled cells in cold-stressed poultry rinsate prepared as described in (B) and run without signal recovery treatment (left panel), with treatment containing 5 mM sodium pyruvate (middle panel), or 200 mM glucose and 20 mM sodium pyruvate.

1. A multi-sample analyte analysis method comprising:
labeling at least one target analyte in each of a plurality of individual samples with at least one unique label probe, wherein:
each unique label probe is configured to bind with a first specific binding agent of a respective target analyte for a respective sample,
each respective first specific binding agent comprises at least one of an antibody, aptamer, lectin, and nucleic acid probe configured to bind to a respective target analyte of a respective sample, and/or is labeled with at least one of a unique synthetic oligonucleotide probe, a protein nucleic acid (PNA) probe, and any other synthetic polynucleotide analog configured for identification of an associated respective sample;
(a) combining the plurality of individual samples into a mixture, and analyzing the combined sample via at least one of an immunoassay, a microfluidic assay, a PCR assay, and a sequencing assay, so as to determine whether any of the samples in the combined sample contain one or more target analyte of interest, or
(b) incubating each sample, wherein incubation optionally includes diluting each sample at least once prior to incubation: optionally washing each sample: optionally treating each washed sample with a crosslinker: combining the plurality of individual samples into a mixture: flowing the mixture within at least one fluidic channel/lane having arranged thereon or in fluid communication with at least one set of a plurality of spatially distinct capture zones, wherein:
each zone is configured with at least one unique oligonucleotide- or PNA-probe complementary to a particular unique label probe of a specific target analyte of at least one specific sample so as to capture the specific target analyte of the at least one specific sample, such that, each distinct capture zone is configured to capture a specific target analyte of the at least one specific sample:
flowing at least one analyte specific fluorescent labeled probes across the set of capture zones, wherein each unique fluorescent labeled probe of the plurality is configured:
to bind with a respective specific target analyte of a respective sample, and comprises at least one of a fluorescent labeled antibody, aptamer, lectin, oligonucleotide probe, PNA probe, and any analyte specific reagent capable of binding to any portion of a respective specific target analyte;
exposing the set of capture zones to:
at least one fluorescing wavelength of light/radiation configured to cause at least one unique fluorescent labeled probe bound to a respective specific target analyte of a respective capture zone and sample to fluoresce: or
a plurality of fluorescing wavelengths of light/radiation configured to cause respective analyte specific fluorescent labeled probes corresponding to a respective fluorescing wavelength and bound to a respective specific target analyte of a respective capture zone and sample to fluoresce:
and
imaging at least one set of capture zones for at least one channel/lane during such fluorescing to produce at least one image thereof; and analyzing the at least one image, or a plurality of image each corresponding to a specific fluorescing wavelength, to determine a number of captured specific target analytes for each zone/sample.

2-24. (canceled)

25. A single use, multiple-sample analyte analysis cartridge device, or lane thereof, comprising:
optionally, a housing configured for insertion and removal from an assay system,
one or more fluidic channels/lanes, optionally arranged along at least a portion of the housing, each fluidic channel/lane configured to receive at least one flow,
wherein:
the at least one flow comprises a mixture of a plurality of individual samples,
each of the individual samples having specific target analytes therein labeled with a unique label probe,
each unique label probe is configured to bind with a first specific binding agent of a respective target analyte for a respective sample,
each respective first specific binding agent comprises at least one of an antibody, aptamer, lectin, and nucleic acid probe configured to bind to a respective target analyte of a respective sample, and/or is labeled with at least one of a unique synthetic oligonucleotide probe, a protein nucleic acid (PNA) probe, and any other synthetic polynucleotide analog configured for identification of an associated respective sample;
at least one set of a plurality of spatially distinct capture zones arranged along at least one fluidic channel/lane, and
each zone is configured with at least one unique oligonucleotide- or PNA-probe complementary to a particular unique label probe of a specific target analyte of at least one specific sample so as to capture the specific target analyte of the at least one specific sample, such that, each distinct capture zone is configured to capture a specific target analyte of the at least one specific sample.

26-39. (canceled)

40. A single use, multiple-sample analyte analysis cartridge device, or lane thereof, comprising:
optionally a housing configured for insertion and removal from an assay system;
a plurality of fluidic channels/lanes optionally arranged along at least a portion of the housing, each fluidic channel/lane configured to receive at least one flow, wherein:
the at least one flow comprises a mixture of a plurality of individual samples, and with each of the individual samples, having specific target analytes therein labeled with a unique label probe,
each unique label probe is configured to bind with a first specific binding agent of a respective target analyte for a respective sample,
each respective first specific binding agent comprises at least one of an antibody, aptamer, lectin, and nucleic acid probe configured to bind to a respective target analyte of a respective sample, and/or is labeled with at least one of a unique synthetic oligonucleotide probe, a protein nucleic acid (PNA) probe, and any other synthetic polynucleotide analog configured for identification of an associated respective sample;
at least one set of a plurality of spatially distinct capture zones arranged along at least one fluidic channel/lane, and
each zone is configured with at least one unique oligonucleotide- or PNA-probe complementary to a particular unique label probe of a specific target analyte of at least one specific sample so as to capture the specific target analyte of the at least one specific respective sample, such that, each distinct capture zone is configured to capture a specific target analyte of the at least one specific sample;
and
one or more windows, each being arranged adjacent a respective capture zone and configured to view at least a portion of a set of the plurality of capture zones, or each respective window configured to view at least a portion of a respective capture zone of a set of capture zones, such that at least one of each set of capture zones and each capture zone can be imaged.

41. (canceled)

42. A multi-sample analyte analysis method comprising:
labeling at least one target analyte in each of a plurality of individual samples with a unique label probe and an analyte specific fluorescent labeled probe,
wherein:
(a) each unique label probe is configured to bind with a first specific binding agent of a respective target analyte for a respective sample, and each respective first specific binding agent:
comprises at least one of an antibody, aptamer, lectin, and nucleic acid probe configured to bind to a respective target analyte of a respective sample, and/or is labeled with at least one of a unique synthetic oligonucleotide probe, a protein nucleic acid (PNA) probe, and any other synthetic polynucleotide analog configured for identification of an associated respective sample,
each unique fluorescent labeled probe of the plurality of individual samples is configured to bind with a respective specific target analyte of a respective sample, and comprises at least one of a fluorescent labeled antibody, aptamer, lectin, oligonucleotide probe, PNA probe, and any analyte specific reagent capable of binding to any portion of a respective specific target analyte;
optionally incubating each sample, incubation optionally includes diluting each sample at least once prior to incubation;
optionally washing each sample;
optionally treating each washed sample with a crosslinker;
combining the plurality of individual samples into a mixture; flowing the mixture within at least one fluidic channel/lane having arranged thereon or in fluid communication with at least one set of a plurality of spatially distinct capture zones, wherein each zone is configured with at least one unique oligonucleotide- or PNA-probe complementary to a particular unique label probe of a specific target analyte of a respective specific sample so as to capture the specific target analyte of the at least one specific sample, such that, each distinct capture zone is configured to capture a specific target analyte of the at least one specific sample,
and
exposing the set of capture zones to at least one fluorescing wavelength of light/radiation configured to cause at least one unique fluorescent labeled probe bound to a respective specific target analyte of a respective capture zone and sample to fluoresce; imaging the set of capture zones during such fluorescing to produce at least one image thereof, and analyzing the at least one image to determine a number of captured specific target analytes for each zone/sample,
or
(b) each unique label probe is configured to bind with a first specific binding agent of a respective target analyte for a respective sample,
each respective first specific binding agent:
comprises at least one of an antibody, aptamer, lectin, and nucleic acid probe configured to bind to a respective target analyte of a respective sample, and/or is labeled with at least one of a unique synthetic oligonucleotide probe, a protein nucleic acid (PNA) probe, and any other synthetic polynucleotide analog configured for identification of an associated respective sample:
combining the plurality of individual samples into a first mixture; and
exposing the first mixture to a plurality of sets of capture particles, thereby forming a second mixture, wherein each set of capture particles is:
configured with at least one unique oligonucleotide- or PNA-probe complementary to a particular unique label probe of a specific target analyte of at least one specific sample so as to capture the specific target analyte of the at least one specific sample, such that, each set of respective capture particles is configured to capture a specific target analyte of the at least one specific sample.

43.-64. (canceled)

65. A multi-sample analyte analysis method comprising:
labeling at least one target analyte in each of a plurality of individual samples with at least one unique label probe,
wherein:
each unique label probe is configured to bind with a first specific binding agent of a respective target analyte for a respective sample, and each respective first specific binding agent:
comprises at least one of an antibody, aptamer, lectin, and nucleic acid probe configured to bind to a respective target analyte of a respective sample, and/or is labeled with at least one of a unique synthetic oligonucleotide probe, a protein nucleic acid (PNA) probe, and any other synthetic polynucleotide analog configured for identification of an associated respective sample;
incubating each sample, wherein incubation optionally includes diluting each sample at least once prior to incubation; optionally washing each sample;
optionally treating each washed sample with a crosslinker; combining the plurality of individual samples into a first mixture; exposing the first mixture to at least one set of capture particles forming a second mixture,
wherein:
each set of capture particles is configured with at least one unique oligonucleotide—or PNA-probe complementary to a particular unique label probe of a specific target analyte of at least one specific sample so as to capture the specific target analyte of the at least one specific sample, such that, each distinct set of capture particles is configured to capture a specific target analyte of the at least one specific sample; exposing at least one of the first mixture and second mixture to at least one analyte specific fluorescent labeled agent,
each unique fluorescent labeled probe of the plurality is configured:
to bind with a respective specific target analyte of at least one sample, and comprises at least one of a fluorescent labeled antibody, aptamer, lectin, oligonucleotide probe, PNA probe, and any analyte specific reagent capable of binding to any portion of a respective specific target analyte;
exposing the second mixture, optionally via flow cytometry, to:
at least one fluorescing wavelength of light/radiation configured to cause at least one unique fluorescent labeled probe bound to a respective specific target analyte to fluoresce; or
a plurality of fluorescing wavelengths of light/radiation configured to cause respective analyte specific fluorescent labeled probes corresponding to a respective fluorescing wavelength and bound to a respective specific target analyte to fluoresce;
analysis of at least one set of the capture particles, optionally via flow cytometry, during such fluorescing to produce at least one data set thereof,
and
analyzing the at least one data set, or a plurality of data sets, each corresponding to a specific fluorescing wavelength, to determine a number of at least one set of capture particles that have captured specific target analytes.

66. A multi-sample analyte analysis method comprising:
combining multiple individual samples into a combined/pooled sample, wherein each individual sample is labeled with a known analyte specific antibody;
pre-screening the combined sample via at least one of an immunoassay, a PCR assay, and a sequencing assay to determine if any of the combined samples contains a target analyte of interest;
and
upon such pre-screening indicating a positive results for the combined sample, flowing the combined sample over a plurality of distinct capture zones, each distinct capture zone being configured to capture a specific target analyte of the combined sample.

67. (canceled)

68. (canceled)

69. A method for performing a ferrofluid-based microfluidic fluorescence in-situ hybridization assay comprising:
preparing a first mixture of ferrofluid and a sample containing or suspected of containing a target analyte;
flowing the first mixture through a channel/lane comprising a capture region while applying a magnetic field, wherein the capture region comprises one or more binding agents which capture the target analyte;
flowing a second solution into the channel/lane, wherein the second solution comprises a labeled probe configured to hybridize with the target analyte;
raising the temperature of the channel/lane to the probe hybridization temperature;
allowing the probe to hybridize with the sample;
flowing the ferrofluid through the channel/lane to remove the unbound probe;
and
measuring for the presence or absence of the label.

70. A method for performing a ferrofluid-based microfluidic fluorescence in-situ hybridization assay comprising:
preparing the first mixture of claim 69;
combining at least the first mixture and a second mixture, wherein the second mixture comprises the labeled probe of claim 1, wherein the probe is allowed to hybridize with the target analyte at the probe hybridization temperature;
flowing the combined mixture through the channel/lane of claim 1;
applying a magnetic field to flow the ferrofluid through the channel/lane to remove unbound probe;
and
measuring for the presence or absence of the label.

71. A method of fluorescence in-situ hybridization detection of an analyte in a sample comprising:
providing the sample of claim 1;
providing the labeled probe of claim 1, wherein said labeled probe is a fluorescent label probe, wherein said fluorescent label probe comprises a binding sequence;
combining at least the sample, the fluorescent label probe, and a buffer to form a mixture;
incubating the mixture at the probe hybridization temperature;
adding the mixture to a ferrofluid-based microfluidic device;
flowing the ferrofluid and moving the mixture toward the capture region of claim 1, applying a magnetic field, and a detector;
and
detecting the presence or absence of the target analyte.

72. The method of claim 69, wherein the labeled probe is fluorescent.

73. The method of claim 69, wherein the probe is a PNA-based probe or a DNA-based probe.

74. (canceled)

75. The method of claim 69, wherein (a) the fluorescent label probe is non-fluorescent and is activated upon hybridizing with the target analyte or (b) the fluorescent label probe is quenched by a complementary sequence comprising a quenching moiety, wherein the complementary sequence is released upon the binding of the fluorescent label probe with the target analyte.

76-78. (canceled)

79. The method of a claim 69, wherein the ferrofluid is a biologically compatible ferrofluid.

80. The method of claim 69, wherein the ferrofluid is a biologically compatible PEGylated ferrofluid or a biologically compatible surfactant stabilized ferrofluid.

81. (canceled)

82. The method of claim 69, wherein the analyte is a bacterial cell, a yeast cell, a mold cell or spore, a parasite, or a parasite oocyst.

83. (canceled)

84. (canceled)

85. The method of claim 69, wherein either the first mixture or second mixture contains Polymyxin B.

86. The method of a claim 69, wherein a quencher dye is used to decrease non-specific signal in the ferrofluid-based FISH assay.

87. (canceled)

88. The method of claim 69, wherein the hybridization time is about 5 minutes or less, about 10 minutes or less, about 15 minutes or less, about 20 minutes or less, about 25 minutes or less, about 30 minutes or less, about 35 minutes or less, about 40 minutes or less, about 45 minutes or less, about 50 minutes or less, about 55 minutes or less, about 60 minutes or less.

89. The method of claim 69, wherein the hybridization time is from about 5 minutes to about 60 minutes, from about 10 minutes to about 60 minutes, from about 15 minutes to about 60 minutes, from about 20 minutes to about 60 minutes, from about 25 minutes to about 60 minutes, from about 30 minutes to about 60 minutes, from about 35 minutes to about 60 minutes, from about 40 minutes to about 60 minutes, from about 45 minutes to about 60 minutes, from about 50 minutes to about 60 minutes, from about 55 minutes to about 60 minutes.

90-96. (canceled)

97. The method of claim 69, wherein the hybridization time is from about 5 minutes to about 20 minutes, from about 10 minutes to about 20 minutes, from about 15 minutes to about 20 minutes.

98. (canceled)

99. The method of claim 69, wherein the hybridization time is from about 5 minutes to about 10 minutes.

100. The method of claim 69, wherein the sample is incubated in media comprising glucose and/or pyruvate prior to analysis.

101. (canceled)

102. (canceled)

103. The method of claim 69, wherein the sample is incubated in media comprising 200 mM glucose and 20 mM pyruvate prior to analysis.

104.-106. (canceled)

107. A kit for performing a ferrofluid-based microfluidic fluorescent in-situ hybridization assay comprising:
a cartridge comprising a plurality of channels/lanes;
an inlet receiving a ferrofluid, biological cell mixture, and a label probe, the inlet in communication with at least one of the plurality of channels/lanes;
a magnetic field source arranged proximate to at least one of the inlet and the plurality of channels/lanes;
at least one capture region having one of more binding agents for capturing at least a first type of analyte, the at least one capture region arranged proximate to a first portion of at least one of the channels/lanes, wherein the first portion is downstream from the inlet;
a detection reagent comprising a fluorescent label probe, wherein said fluorescent label probe comprises a binding sequence, configured to hybridize with the target analyte.

108. (canceled)

109. (canceled)


SEQUENCE LISTING (16 sequences)

SEQ ID NO: 1 - RNA, 28 nt, Unknown
  16S rRNA
uaacgcguuaaguagaccgccuggggag

SEQ ID NO: 2 - DNA, 11 bp, Artificial Sequence
  Salmonella probe
  misc_feature (1)..(1): Alexa Fluor-488 dye attached with O-methylation
gtctacttaac

SEQ ID NO: 3 - DNA, 15 bp, Artificial Sequence
  Helper probe 1
gttttaaccttgcgg

SEQ ID NO: 4 - DNA, 12 bp, Artificial Sequence
  Helper probe 2
cgcgttagctcc

SEQ ID NO: 5 - DNA, 11 bp, Artificial Sequence
  Salmonella probe
  misc_feature (11)..(11): Alexa Fluor-488 dye attached via lysine residue
gtctacttaac

SEQ ID NO: 6 - DNA, 24 bp, Artificial Sequence
  ZipCode-1 oligonucleotide
  misc_feature (1)..(1): conjugated to 5′-linked biotin attached to a 15-atom mixed polarity triethylene glycol spacer (5BioTEG) and an 18-atom hexa-ethylene glycol spacer (iSP18)
tgcgacctcagcatcgacctcagc

SEQ ID NO: 7 - DNA, 24 bp, Artificial Sequence
  ZipCode-5 oligonucleotide
  misc_feature (1)..(1): conjugated to 5′-linked biotin attached to a 15-atom mixed polarity triethylene glycol spacer (5BioTEG) and an 18-atom hexa-ethylene glycol spacer (iSP18)
gaccaccttgcgatcgggtacagc

SEQ ID NO: 8 - DNA, 24 bp, Artificial Sequence
  ZipCode-13 oligonucleotide
  misc_feature (1)..(1): conjugated to 5′-linked biotin attached to a 15-atom mixed polarity triethylene glycol spacer (5BioTEG) and an 18-atom hexa-ethylene glycol spacer (iSP18)
cagcggtagaccacctatcgtgcg

SEQ ID NO: 9 - DNA, 24 bp, Artificial Sequence
  ZipCode-25 oligonucleotide
  misc_feature (1)..(1): conjugated to 5′-linked biotin attached to a 15-atom mixed polarity triethylene glycol spacer (5BioTEG) and an 18-atom hexa-ethylene glycol spacer (iSP18)
gaccatcgtgcgggtaggtagacc

SEQ ID NO: 10 - DNA, 24 bp, Artificial Sequence
  AntiZipCode-1 oligonucleotide
  misc_feature (1)..(1): conjugated to 5′ amino modifier and 6-carbon spacer arm (5AmMC6) and an 18-atom hexa-ethylene glycol spacer (iSP18)
gctgaggtcgatgctgaggtcgca

SEQ ID NO: 11 - DNA, 24 bp, Artificial Sequence
  AntiZipCode-5 oligonucleotide
  misc_feature (1)..(1): conjugated to 5′ amino modifier and 6-carbon spacer arm (5AmMC6) and an 18-atom hexa-ethylene glycol spacer (iSP18)
gctgtacccgatcgcaaggtggtc

SEQ ID NO: 12 - DNA, 24 bp, Artificial Sequence
  AntiZipCode-13 oligonucleotide
  misc_feature (1)..(1): conjugated to 5′ amino modifier and 6-carbon spacer arm (5AmMC6) and an 18-atom hexa-ethylene glycol spacer (iSP18)
cgcacgataggtggtctaccgctg

SEQ ID NO: 13 - DNA, 24 bp, Artificial Sequence
  AntiZipCode-25
  misc_feature (1)..(1): conjugated to 5′ amino modifier and 6-carbon spacer arm (5AmMC6) and an 18-atom hexa-ethylene glycol spacer (iSP18)
ggtctacctacccgcacgatggtc

SEQ ID NO: 14 - DNA, 13 bp, Artificial Sequence
  Lis240D oligonucleotide
  misc_feature (1)..(1): Alexa Fluor-488 dye attached
tagccgaaaccat

SEQ ID NO: 15 - DNA, 13 bp, Artificial Sequence
  LisUn-3 H2 oligonucleotide
  misc_feature (1)..(1): two solubility enhancer groups attached
  misc_feature (13)..(13): two solubility enhancer groups attached
attcggtattagc

SEQ ID NO: 16 - DNA, 8 bp, Artificial Sequence
  LIS240D-DQ oligonucleotide
  misc_feature (8)..(8): Dabcyl attached
ttcggcta